(19) United States
(12) Patent Application Publication
(10) Pub. No.: US 2025/0044271 A1
(43) Pub. Date: Feb. 6, 2025

(54) A COMPUTER-IMPLEMENTED METHOD OF PREDICTING QUALITY OF A FOOD PRODUCT SAMPLE

(71) Applicant: Mars, Incorporated, McLean, VA (US)

(72) Inventor: Jorn BROUWERS, Chicago, IL (US)

(21) Appl. No.: 18/718,711

(22) PCT Filed: Dec. 12, 2022
(86) PCT No.: PCT/US2022/052504

§ 371 Date: Jun. 12, 2024

(30) Foreign Application Priority Data
Dec. 13, 2021 (GB) 2118033.6

(51) Int. Cl. G01N 33/02; G06N 3/0455; G06N 3/09

(52) U.S. Cl. CPC G01N 33/02; G06N 3/0455; G06N 3/09

(57) ABSTRACT

A computer-implemented method of predicting quality of a food product sample after a mixing process, based on properties of the food product, comprises: building a hybrid model by: training an autoencoder in an unsupervised learning step using historical process data of food product samples; training a supervised model in a supervised learning step using the output of the autoencoder; and predicting the quality of the food product by inputting process data of current samples into the hybrid model and classifying the samples.

### CROSS-REFERENCE TO RELATED APPLICATION(S)

This is a National Stage Application under 35 U.S.C. § 371 of International Application No. PCT/US2022/052504, filed on Dec. 12, 2022, which claims priority to G.B. Patent Application No. 2118033.6, filed on Dec. 13, 2021, the entireties of which are incorporated herein by reference.

### FIELD OF THE INVENTION

The present invention relates to methods of predicting quality of food samples using process data.

### BACKGROUND OF THE INVENTION

Quality is defined as the group of those product characteristics that satisfy explicit and implicit customer requirements (Scipioni, Saccarola, Centazzo, & Arena, 2002). The quality of a product is seen as one of the most important elements for every organization that offers goods. Consumers require the quality of the products they consume to be constant, particularly if the product is marketed associated with a brand (Cano Marchal, Gómez Ortega, & Gamez Garcia, 2019). If the quality of the product or service fluctuates unsteadily, consumers may not know what to expect and would stop buying the unreliable products (Savov & Kouzmanov, 2009). Therefore, companies need to develop and approve high standards to produce and sell a product within a standardized process.

Literature in the food industry distinguishes different types of food product quality. The first aspect concerns the food safety which are the compulsory requirements for selling a food. Subjective quality is user-oriented and concerns how quality is perceived by the consumers and how this might attract consumers, whereas objective quality refers to the physical characteristics created in the product by the engineers and food technologists (Lim & Jiju, 2019; Scipioni et al., 2002). The objective quality further relates to the product oriented and process-oriented quality. Product-oriented quality concerns the product's physical properties like fat percentage and viscosity, whereas process-oriented quality relates to the degree where quality characteristics of the products maintain a product stable between specification limits (Lim & Jiju, 2019).

Food quality management is, compared to other industries, challenging due to the complex character of food products together with the unpredictable and evolving behaviour of people involved in the food chain (Lim & Jiju, 2019). Variability of the raw material properties is one of the distinctive characteristics of the food industry, which significantly influences the quality of the final products (Cano Marchal et al., 2019). The high variability of input can induce a major source of process disturbances and is caused by a wide variety of reasons. Differences between different producers or even different lots of the same producer and biological variation cause variability within the raw materials. In addition, the perishability of materials also deteriorates the quality of raw materials.

Another distinctive character is the food products itself, which are complex substances whose properties are difficult to measure online and make control even more difficult. Moreover, the food process consists of many different couplings between the process. Each disturbance is easily propagated throughout the process, which in turn affects the quality of the final food product. As a result, one of the main objectives of food processing operations is to damp the variability of the inputs, such that consistent objective quality is obtained (Cano Marchal et al., 2019).

### 1.1 Research Motivation

This study is conducted at Mars Nederland B.V, which is part of Mars Incorporated. Mars is one of the most prominent producers of chocolates, confectionery, food, and pet care products in the world. Within Mars Incorporated, five principles form the foundation of how business is performed. These five principles are put at the centre of every decision made and include quality, responsibility, mutuality, efficiency and freedom. The quality pillar implies that Mars is committed to gaining the highest quality of its work. Mars states quality is the standard for actions and the source for a reputation of high standards. It delivers customer satisfaction through offering consumers the best buy for their needs. As a result, Mars is continuously seeking for new ways to improve their products and processes in an efficient manner.

The manufacturing location in Veghel has the largest production volume of all Mars factories and is one of the largest chocolate factories in the world. Each hour one million chocolate bars are produced, which are delivered to more than 80 countries around the world. The scope of this study focuses on—but is not limited to—the conche machine, which produces semi-finished chocolate in a closed system. Conching is a fully automated mixing process that evenly distributes cacao-butter within chocolate. The chocolate production in Veghel is relatively traditional. As such, this work makes a contribution to modernization of the chocolate production industry, i.e., to improve the production process and ensure the quality of chocolate.

As explained, quality is one of the five principles of Mars, meaning that Mars is committed to gain the highest quality of its work. Although conching is a fully automated process, still deviations in the physical properties of chocolate occur. These deviations are propagated throughout the production process and affect the final quality of the chocolate bars and the efficiency of the production plant. Mars monitors its chocolate production process using four physical properties and classifies a batch of chocolate as Right First Time (RFT) if all four physical properties are within specification limits. Within Mars, it is stated the product variability in batches remains a challenge and it is indicated that in previous years a fraction of all batches required a manual adjustment. This manual adjustment often includes adding the expensive raw materials or extending production time. As an example, in terms of costs, over the last 5 years an over usage of only one Raw Material for the manufacturing plant in Veghel costs annually a significant amount. From the actual Performance per milling group it can be observed that large differences among different milling groups exist. Certain milling groups perform worse due to the frequent switching of recipes.

This variability in the semi-finished chocolate properties cause problems at the downstream manufacturing lines. Mars observes problems during the production or packaging of the chocolate bars which are related to the chocolate properties. These problems include too heavy bars, bars with visible fillings, too high bars or bars with too wide bottoms. During the production of chocolate, viscosity and yield stress are the main qualitative properties attempted to steer on. Chocolate with too high viscosity causes the bars to weigh too heavy and as a result a lot of vibrations are required to remove the redundant chocolate of the bars. Besides, a too high viscosity enhances the probability of seeing the filling of a bar. Visible fillings occur because the chocolate does not flow through and then bars are seen as waste. Chocolate with a too high yield cause high covering/decorations of chocolate bars. Due to the high decorations, problems arise in the packing room and the production lines risk to be stopped. Cooling the bars is the final step before packaging. In case the yield is too low, the chocolate continues floating during the cooling and as a result, the bars have too little decoration and too wide bottoms.

Chocolate production is known as a process where crystallisation is applied. In such situations online monitoring and process control is known to be challenging (P. J. Cullen & O'donnell, 2009). Similar for Mars, its current chocolate production control is either reactive or relies heavily on the judgement of operators. At the end of the production cycle the viscosity, yield, fat content and moisture of the chocolate batch is measured using laboratory equipment. As a result, Mars can only extend or adapt the production process with certainty once the incorrect properties are known. Therefore, the measuring properties at the end of the production cycle can delay the production process. However, in few cases in an early production phase the operators are able to detect the machine is not approaching the ideal behaviour. In such case the operator is able to intervene the process by adding either adding a manual dosage of raw materials or by extending the duration of the different conche phases. The choice and correctness of the intervention totally depends on the judgement of the operator. Thereupon, the conching process can be seen as a large black box, with unknown effects of the inputs on the output.

Accordingly, literature states chocolate manufacturers require an efficient, reliable and a prompt method for product and quality control (Stohner, Zucchetti, Deuber, & Hobi, 2012).

Summarizing the problem, the production of chocolate is known to be complex and challenging. The variability in the physical properties of the semi-finished chocolate is propagated throughout the production process. It affects the efficiency of the manufacturing plant and the final quality of the chocolate confectionery. This study attempts to open up the black box of the chocolate production and tries to increase the chocolate production process understanding. It explores how current data can be utilized such that consistent objective chocolate quality can be obtained.

### 1.2 Contribution to the Scientific Literature

This study investigated the possibilities of machine learning techniques to apply within the chocolate production process. The contributions to the literature are listed as follows: The chocolate production is a traditional industry where, machine learning applications in the chocolate production industry are sparse. Existing methods require investments of capital sensors or require manual sampling which are not suitable for online process monitoring. This research tries to investigate how machine learning can be applied using readily available and low costs data.
The research further contributes to the literature by applying anomaly detection methods in a new industrial domain. Current applications of anomaly detection are diverse and include engine faults detection, fraud detection, medical domains, cloud, monitoring or network intrusions detection. This research applies the semi-supervised anomaly detection methods during the production of chocolate.
Additionally, the research contributes to the anomaly detection literature by investigating how the output of different unsupervised autoencoders can be used as input to supervised learning algorithms. Current literature uses the latent vector or reconstruction error vector generated by convolutional or LSTM autoencoders as input to support vector machines. Contrary we show, for this application, the reconstruction error of an attention based autoencoder can best be combined with a random forest to detect faults.

### 1.3 Research Objective

The goal of this study is to explore how and which machine learning techniques can be applied to enhance production control, including production control in chocolate manufacture. The data-driven approach is chosen due to the fact that Mars and other producers store a large amount of data at different systems without using this data to its full potential. If built and implemented correctly, the model can provide additional insight in the production process by exploring relations in the unlabelled and unstructured production data.

### 1.4 Research Questions

Based on the research objectives and the problem description the main research question that will be central to this study is:

What machine learning model can be developed to learn the influential factors of the quality during the production of Chocolate?

In order to thoroughly answer the research question, the problem is approached by answering the following sub questions:
1. What is Mars' current practice for monitoring the quality of chocolate?
2. Which methods are proposed in the literature regarding data-driven food quality control?
3. Which features can be defined for chocolate which potentially influence the final chocolate quality?
4. How to utilize the production data and formulate a data-driven model to predict the chocolate quality, which maximizes both predictive performance and interpretability?
5. How can the predictive model be used during the chocolate quality production?
1.5 Scope

The inventor's intention was to explore the possibilities to apply artificial intelligence during the manufacturing of chocolate confectioneries. The study can be seen as a proof of concept to make the chocolate confectionery more intelligent. The following summarizes the reasoning to scope the project to the chocolate production on the conches:
This research fits the current trend of automation and data exchange in manufacturing, known as Industry 4.0, indicating data availability is one of the most important factors when defining the scope. Therefore, this research focuses will focus on the chocolate production step of Mars where most data are easily and readily available.
Mars notices problems during the production of chocolate bars. The problems at the chocolate bar productions lines are often related to the deviations in properties of the chocolate. However, the actual impact and costs of these deviations is left out of scope. As a result, no model's evaluation in terms of saved costs is performed.
Although Mars produces multiple chocolate recipes, for this research it is chosen to focus exclusively on the Recipe A recipe produced on conches from Milling Group Type A 3. Only utilizing conches from Type A 3 ensures differences between machines are limited. Additionally, this contributes to obtaining the largest possible sample size.

Physical, surface and sensory quality are the three types of chocolate quality. Mars only measures the physical chocolate quality. Consequently it is chosen to scope the project on the physical chocolate quality.
The implementation of the new predictive model in the day-to-day business of Mars is not included in this study.

### 2 Literature

This section first discusses relevant literature regarding food mixing processes which are related to the control or monitor of chocolate production. Specifically, the literature review identifies the possible methods and its current limitations. Based on the limitations of existing techniques, the literature further explores available machine learning techniques applicable for controlling the chocolate production process. Finally, this Chapter reviews on available anomaly detection methods which might also be applicable for the observed problem.

### 2.1 Controlling Food Mixing Processes

Currently, there is an increasing trend in the food mixing industry to adopt a Process Analytical Technology (P. J. Cullen & O'donnell, 2009; P. Cullen, Bakalis, & Sullivan, 2017). Its goal is to shift from a paradigm of testing quality in post manufacture to designing quality during manufacture. Designing quality during the production can be achieved by fundamental process understanding or real time monitoring the critical product quality properties (P. J. Cullen & O'donnell, 2009). Monitoring and control of the mixing processes in the food industry, such as the chocolate production, is critical. Incomplete or over-mixing of a product may result in product separation, attrition and undesirable product texture (P. J. Cullen & O'donnell, 2009). In some food applications, the effects of mixing can last long after the mixing operation has ceased, and it may take a long time to reach the end point. Especially processes involving crystallisation are known where the effects of mixing can continue even after agitation has stopped. Chocolate making is one application where crystallisation is used. In such situations, (on-line) monitoring and process control can be very challenging. As a result, mixing will be a source of variability within the manufacturing process (P. J. Cullen & O'donnell, 2009).

### 2.1.1 Methods

In order to damp the variability occurred in mixing processes, several monitoring techniques are available. On a high level, monitoring the quality of the food processing can be distinguished into at-line, on-line and in-line analysis. At line analysis requires taking a manual material sample, while online and inline monitor techniques allow for automated data collection as they do not require manual sampling (Bowler, Bakalis, & Watson, 2020b). As such, the latter two are considered as more suitable for real time process monitoring. Online methods automatically take samples to be analysed without stopping, whereas inline methods directly measure the process stream without sample removal. This section provides an overview of monitoring techniques applicable to food mixing processes.

### 2.1.1.1 At-line Sampling

In food mixing applications it is often not possible to determine the whole mixture at a single point in time. In such situations, at-line sampling is a technique often used as a method to assess the state of mixedness (Rielly, 2015). Rheology measurements, such as viscosity, is an example for which the property is usually assessed using an offline laboratory machine. In order to obtain a comprehensive understanding of the whole mass, multiple samples from different locations are required (P. Cullen et al., 2017). Another disadvantage from sampling is that it is a reactive activity and does not facilitate preventive activities (Lim & Jiju, 2019). However, many of food processes do not have the possibility to any sampling at all. Either because analyzers are not available or they are simply too expensive. In these circumstances, it is often the expert operators that play the role of at-line sensor, assessing the quality of the final products based on their experience (Cano Marchal et al., 2019). Their expert knowledge enables experienced engineers to detect anomalous patterns during the food production. As a result, in these cases it is crucial to have a rich understanding of the behavioral characteristic of the food production process.

### 2.1.1.2 Phenomenological Models

Despite the pervasiveness of mixing processes and the vast quantities of materials mixed every day, mixing processes are still not fully understood scientifically (P. J. Cullen & O'donnell, 2009). However, González et al. (2020) and González, Acosta, Camilo, Rivas, and Muñoz (2021) made an attempt by developing phenomenological models to predict qualitative properties of chocolate. A phenomenological model is a scientific model which describes the empirical relationship of phenomena. The relationship is consistent with fundamental theory, but not directly derived from first principles. It simply describes how variables interact and not why.

First, González et al. (2020) proposed a phenomenological model to predict the conching degree, which is an indicator of the sensory quality of chocolate. In order to reduce operating time while guaranteeing the desired chocolate quality, their model tries to provide understanding in the phenomena of the dynamic behaviour of the conching process. Using their predictive model, they propose to reduce the conching time from 750 to 630 minutes, which does not significantly modify the taste and smell of chocolate but does result in a capacity optimization of 100 hours per month. The phenomenological model requires complex experimental techniques to quantify the model input variables, which limits its applicability for online process control. The authors suggest the use of virtual sensors for process control and the use of easily available measured variables in real-time. Several available sensor techniques which might be applicable are explained in the next section. In a later study, González et al. (2021) use the phenomenological based semi-physical model for predicting the chocolate structural changes during the conching process. They state within the chocolate industry a model to predict the dynamics of rheological variables, such as viscosity, is currently unavailable. The created model accurately predicts viscosity and can be used to propose possible process modifications, while guaranteeing rheological quality. The prediction of structural changes in chocolate using a model will reduce experimentation in the plant.

### 2.1.1.3 Sensor Techniques

Sensor data is considered as the most relevant data source for data generation. Sensor data should be combined with timestamps and offline stored in order to generate time series. Additionally, the authors stress the importance of storing domain knowledge (Stahmann & Rieger, 2021). Considerable research has been performed to incorporate sensor methods for food processing operations. Sensor applications facilitate inline and online measurements of several key variables during the food mixing process (Cano Marchal et al., 2019). (P. J. Cullen & O'donnell, 2009) reviewed how different sensors can provide insights into the complex mechanisms of mixing and can contribute to effective control in the food industries. The techniques applicable to the chocolate production are summarized below. First, simple and low cost applications of sensor techniques are explained. Afterwards, the more advanced techniques, driven by the recent development in computer data acquisition and treatment methods are summarized. These advanced techniques enable detailed in-line and online analysis of food mixing processes (P. J. Cullen & O'donnell, 2009; P. Cullen et al., 2017; Bowler, Bakalis, & Watson, 2020a).

### Temperature and Pressure

Temperature and pressure are simple sensor measurements for food mixing systems. Defining thresholds for such sensor measurements, is a rather simple way to monitor the production process. Once the specified threshold is violated, an automatic warning system generates an alarm. Implementing such a system reduces the manually monitoring time, but could provide, especially in complex domains such as chocolate production, many false alerts (Cano Marchal et al., 2019). As a result it is hard to detect these failures using solely thresholds as food product failures require to take into account joint characteristics of multiple channels.

### Power and Torque Measurements

The power draw and torque measurements are also simple and low costs techniques, which can be used to determine the force required to turn the mixing blades. Both are seen as one of the most fundamental measurements of mixing. These techniques are capable of characterizing the mixing system as they can be used as an indication whenever the rheology changes (P. J. Cullen & O'donnell, 2009; P. Cullen et al., 2017). As an example, the torque and power draw can be used as an alternative technique to predict viscosity instead of measuring on an off-line laboratory meter. Real-time process monitoring based on either the power or torque measurements can facilitate preventive interventions. Simple torque measurements are already utilized to characterize the behaviour of dough during processing. The peak torque from a mixing trace, seems to correlate with the actual performance measurements (P. J. Cullen & O'donnell, 2009).

### Flow Measurements

The complex flow inside a vessel can be measured used single-point and whole-field techniques. Singlepoint measuring techniques, such as hot-wire, laser and phase Doppler, determine the velocity at a given point inside the vessel. Particle image velocimetry and planar laser-induced velocimetry are whole-field techniques which determine the flow pattern inside a wider region. Flow mapping within stirred vessels may provide useful insights into the mixing process, but may not be suitable for process monitoring or control of chocolate production as many require transparency of particles (P. Cullen et al., 2017).

### Near Infrared Spectroscopy

As explained above, mixing of dough can be monitored online using torque sensors. It was found the extent of mixing has critical impact on the final dough quality. During the mixing a range of physicochemical changes occur. Near infrared spectroscopy is another sensor technique capable of providing valuable information on the extent of mixing (P. J. Cullen & O'donnell, 2009). The pharmaceutical industry already successfully implemented Near Infrared spectroscopy as an inline monitoring technique to measure product moisture, ingredient identity and homogeneity. The food mixing industry and pharmaceutical industry both face the same challenge of ensuring homogeneity in their mixtures. For the pharmaceutical industry, near infrared spectroscopy is even considered as most advanced and most promising techniques (P. Cullen et al., 2017).

### Imaging

Chemical Imaging is another technique which can be used to describe ingredient concentration and distribution in heterogeneous solids, semi-solids, powder, suspensions and liquids (Gowen et al. 2008). The technique integrates conventional imaging and spectroscopy to attain spatial and spectral information. It has great potential for monitoring the mixing of food powder of fluid systems because it was already successfully applied for the analysis of complex materials such as pharmaceutical tablets (P. J. Cullen & O'donnell, 2009). Imaging techniques, specific those which can identify the chemical composition, enhance controlling the process and gaining mechanistic insights (P. Cullen et al., 2017). Another well known imaging technique is Magnetic Resonance Imaging (MRI). MRI is a spectroscopic technique based on the interaction between nuclear magnetic moments and external magnetic fields. The technique is capable of obtaining concentration and velocity profiles. MRI has a lot of potential for mixing as it can operate in real-time. However, MRI is not suitable for the production of chocolate as it may only be used for opaque fluids or fine powders (P. Cullen et al., 2017).

### Electrical Tomography

More recently, the applications of electrical tomography technique for process design, understanding and monitoring in Food mixing increased (P. Cullen et al., 2017). Electrical tomography measures the electrical property of a fluid. Examples include the resistance and capacitance of fluids inside a mixing vessel. The technique uses a set of electrodes mounted on the inside of the mixing vessel to measure a certain property. Responses of the sensors are combined into a tomograms and provide information about the flow inside the vessel. Electrical impedance, electrical capacitance and electrical resistance are the available electrical tomography approaches. Such tomographic techniques can be used to monitor and control mixing processes (V. Mosorov, 2015). As an example, the electrical resistance tomography can be used to monitor the mixing rate of a complex suspension within a stirred vessel (Kazemzadeh et al., 2017). Additionally, monitoring electrical capacitance tomography can be used to prevent issues related to rheological properties such as poor mixing, low heat transfer and fouling (P. Cullen et al., 2017).

### 2.1.2 Discussion

As explained, the goal within the food mixing industry is to shift from testing quality in post manufacturing to design quality during manufacturer. All available sensor techniques explained above, can be utilized to real-time monitor the food mixing processes. However, for the practical control in the food industry P. J. Cullen and O'donnell (2009) state techniques should be as simple as possible, affordable and non-invasive. Developing phenomenological models is not a simple task and the advanced sensor techniques may require large investments. As a result, these mixing monitoring techniques may not be applicable in large scaled production sites with multiple machines, such as Mars Veghel. Alternatively, machine learning could also be an innovative technique which could design quality during manufacture. Machine learning utilizes available data sources and can be tailored to a specific task. It is an attractive data analysis method which does not require the challenging development of first-principle models (Simeon, Woolley, Escrig & Watson, 2020).

### 2.2 Machine Learning

In recent years, digitization gave rise to large amounts of data and analyzing this data could enhance process understanding and efficiency. An overall framework of data analytics capabilities in manufacturing processes is visualized in FIG. 2. The framework summarizes all possible sub-problems within manufacturing capable to be solved by big data analytics. Big Data Analytics applies data mining techniques and statistical analysis to utilize the potential value of big data (Belhadi, Zkik, Cherrafi, Yusof, & El fezazi, 2019). The authors of the framework state quality and process control is one of manufacturing processes with the greatest potential value to catch with machine learning. The increase of data availability together with the higher computing power, increasing reliability and easier implementation led to a large increase in the use of machine learning in the food industry over the past five years (Krauß, Frye, Teodoro, Beck, & Schmitt, 2019; Mavani et al., 2021). It can be used in solving complicated tasks and does not require knowledge of pre-existing equations and formulas, such as first-principle models (Mavani et al., 2021; Bowler et al., 2020a). As a result, the number of applications of machine learning is expected to rise even further.

Belhadi et al. (2019) categorizes big data analytics into descriptive, inquisitive, predictive and prescriptive analytics. Descriptive analytics explain the current state of a business situation (Belhadi et al., 2019). It concerns the question ‘what happened?’ or alerts on what is going to happen. Examples of descriptive analytics includes monitoring the mixing process, as explained in Section 2.1, with statistics or visualizations on dashboards. Inquisitive analytics explain why ‘why something happened?’. It seeks to reveal potential rules, characteristics or relationships that exist in the data (Belhadi et al., 2019). Typical examples of inquisitive analysis include clustering analytics, generalization, sequence pattern mining and decision trees. Predictive analytics is a step further, which aims to provide insights in ‘what is likely to happen?’. Historical and current data and machine learning models are used to forecast what will happen (Belhadi et al., 2019). Predictive analytics can further be divided into statistical oriented analytics and knowledge discovery technique (Cheng, Chen, Sun, Zhang, & Tao, 2018). The first technique often uses mathematical models to analyse and predict the data. Mathematical model, such as regression models, often depend on statistical assumptions to be sound. Contrary, the second category is data-driven and does not require any assumptions. This category mainly includes machine learning techniques such as Neural Networks and Support Vector Machines (Belhadi et al., 2019). The fourth analytical level answers the ‘what should be done’ question. Prescriptive analytics tries to improve the process or task at hand based on the output information of the predictive models (Belhadi et al., 2019). Machine learning can be used in all four analytical levels, but is mostly used during the inquisitive and predictive phase. Section 2.2.2 summarizes how these techniques have been applied in the food (mixing) field.

### 2.2.1 Machine Learning Types

Machine learning can be divided into three different categories: supervised learning, unsupervised learning and reinforcement learning (Ge, Song, Ding, & Huang, 2017). The actual category depends on the feedback of the learning system (Alpaydin, 2014). Learning in which data consists of samples inputs along with corresponding labels and for which the goal is to learn a general set of rules that maps the input to the output is known as supervised learning (Bowler et al., 2020a). Supervised learning can be divided into a classification or regression problem. Classifying faults in different categories is a typical example of a classification supervised learning problem, whereas a typical data regression problem concerns the prediction of the key performance of the process (Ge et al., 2017; Mavani et al., 2021). Supervised learning algorithms are applied due to the data-rich, but knowledge-sparse nature of the problem (Wuest, Weimer, Irgens, & Thoben, 2016). Unsupervised learning uses data that consists of samples without any corresponding label. The goal for unsupervised learning is to identify structures among the unlabelled data (Ge et al., 2017; Wuest et al., 2016; Mavani et al., 2021). No feedback is given since unsupervised learning concerns unlabelled data. Examples of unsupervised learning include the discovery of groups of similar examples, determining the distribution of the data or reduce the dimensionality of the data. It is possible to combine supervised and unsupervised learning into Semi-supervised learning. Then a small amount of labelled data is combined with a large amount of unlabelled data. This is especially useful if the labelling costs are too high (Ge et al., 2017). A reinforcement model interacts with an environment in order to learn a given task or goal. Reinforcement learning is a different type of learning as not the proper action is given as feedback, but an evaluation of the chosen action is given by the learning system (Wuest et al., 2016; Mavani et al., 2021). The next section will briefly summarize how these techniques have already been applied in the (chocolate) manufacturing field.

### 2.2.2 Supervised Learning in Food Manufacturing

The primary step in choosing the appropriate machine learning method concerns defining the objective of using Al in their research (Mavani et al., 2021). Regression, classification, quality control and detection are found to be common objectives of Al applications in the food industry. Given sufficient labeled examples, supervised learning models can be designed in such a way that it can facilitate quality control. In this section an overview of the available supervised learning applicable for the food (mixing) industry is given.

### 2.2.2.1 Chocolate Manufacturing

The semi-finished chocolate is a complex substance, whose properties of interest are difficult to measure online. As a result, fast accurate measurements of the properties of interest are usually not an easy task. Most often, well-established laboratory methods are used to determine with sufficient accuracy the values of these properties. (Cano Marchal et al., 2019). However, being able to robustly and accurately obtain values of these properties in an online manner is usually a quite challenging problem (Huang, Kangas, & Rasco, 2007). Although data analytics and machine learning are widely used in other fields, only one research is performed in the field of chocolate making. Therefore, this work can help conching become more intelligent.

To the best of the inventor's knowledge, Gunaratne et al. (2019) is the first and only research which applied machine learning to predict the properties of liquid chocolate, similar to the production at Mars. Using Near-Infrared Spectroscopy data the psycho-chemical quality and sensory properties of chocolate is accurately predicted. Their proposed model uses two neural networks, in which the first use the Near-Infrared Spectroscopy data of samples to predict psycho-chemical data such as viscosity, pH, Brix and colour. The psycho-chemical predictions of the first model are then used as inputs in a second neural network to predict the sensory descriptors of chocolate. However, their proposed model requires near-infrared spectroscopy measurements of samples to predict viscosity and other psycho-chemical properties and is thus not suitable for online monitoring. Additionally, in order to have inline measurements the technique requires a large investment (Gunaratne et al., 2019). In a different setting, Benković et al. (2015) developed an artificial neural network which predicts the effect of different parameter changes on the physical and chemical properties of cacao powder samples. The authors analyze the effect of added water, agglomeration duration, fat content, sweeteners content, bulking agent content on several physical and chemical properties. The MLP network predicts sauter diameter, bulk density, porosity, chroma, wettability and solubility of the chocolate samples. Due to the limited machine learning applications during the chocolate production it is chosen to explore other food industries.

### 2.2.2.2 Other Food Processing Applications

Early in 1995, Ruan, Almaer, and Zhang successfully deployed an Artificial Neural Network to predict the rheology properties of cookie dough batches. Similar to the conching process at Mars, the rheological properties of dough were determined at the end of the batching process which is right after mixing. The required work input, captured by the engine torque curve, relates to the final cookie dough quality. However, the precise relationship is unclear as it is a non-linear and complex problem. An artificial neural network seems to be capable to quantitatively analyze dough rheological properties based on power consumption characteristics. Two years later, Ruan, Almaer, Zou, and Chen proposed an efficient pre-processing method to overcome the main difficulties of handling raw power consumption data with artificial neural networks. As a result of the batching process, the raw data consisted of extreme noisiness, unequal mixing lengths, uncertain starting and stopping points and discontinuity of the curves. Their new method threatens mixing power consumption curves with fast Fourier transform and power spectral density to reduce the noise and size of the data set, before feeding it to the neural network.

Li, Lin, Yu, Wilson, and Young (2021) employed a Long Short-Term memory to detect the pH value during the production of cream cheese. Cream cheese is complex product made from milk and cream, and its pH value influences both texture and flavour of product. During cream cheese fermentation the pH value is decreased over time and accurate prediction allows to stop at the right time. Creating a fundamental model is difficult due to the complexity of cheese. Therefore, machine learning combined with a physical-based kinetic model is used to predict the pH value. The little requirement of knowledge in domain-specific information about the biological-chemical process is considered as a major advantage of using machine learning. Bowler et al. (2020a) developed both classification and regression machine learning models for two laboratory mixing systems. Their models were tested on a honey-water blending and flour-water batter mixing systems and show how ultrasonic sensors can be used to monitor mixing processes. Classification models predict whether the materials were mixed or not, the time until mixing completed is predicted using regression. The authors tested Artificial neural networks, support vector machines, long short-term memory and convolutional neural networks. Results showed different approaches performed best on each prediction task. For classifying the mixture state of honey-water, the use of time domain in LSTMs and CNNs performed better than normal artificial neural networks. Convolutional neural networks showed best performance in predicting the remaining mixing time. Ultrasound sensors are low-cost, real-time, in-line, and capable of operating in opaque systems. Unfortunately, this technique is not applicable for chocolate production as the mass is not opaque. Additionally, their approach is only tested in a laboratory system and not in a large scale mixing system. Due to overfitting support vector machines showed worst performance for all prediction tasks. It must be mentioned, Bowler et al. (2020a) argue whether the good performance can also be achieved in a large scaled industrial setting. In such case, retrieving labels are typically conducted off-line and require time and manual operation, therefore good qualitative labels are often unavailable.

Omari, Behroozi-Khazaei, and Sharifian (2018) used artificial neural networks to model the mushroom drying process in a microwave-hot air dryer. The model predicts the moisture content during the drying process using hot air temperature and the microwave power density. Their model shows drying time can be decreased with increasing the microwave power and air temperature. Developing the dynamic model in this study for predicting the moisture content and adjusting the microwave power based on the moisture content would facilitate online microwave power control. In a similar study, Ardabili et al. (2020) show how using a Radial Basis Function neural network instead of an Multi layered perceptron network achieves even better performance in terms of predicting mushroom temperature variety. The temperature variation of the mushroom growing room was modelled by multi-layered perceptron and radial basis function networks based on independent parameters including ambient temperature, water temperature, fresh air and circulation air dampers, and water tap.

### 2.2.3 Discussion

The supervised learning applications, as described in Section 2.2.2, perform supervised learning tasks within the food industry. However, supervised learning requires sufficient and qualitative labeled examples. For large scaled industrial plants, Bai, Xie, Wang, Zhang, and Li (2021) propose to use semisupervised learning techniques because qualitative labels are often lacking (Bowler et al., 2020a). Pattern recognition can be an alternative tool to conduct quality control (Jiménez-Carvelo, González-Casado, Bagur-González, & Cuadros-Rodríguez, 2019). Anomaly detection is the research area where often little labeled data is available. It focuses on detecting samples which deviate from normal behaviour. Anomaly detection can be a solution to detect incorrect processes and shows great potential to improve the operational stability of industrial processes in various applications (P. Park, Di Marco, Shin, & Bang, 2019).

### 2.3 Anomaly Detection

Anomaly detection methods enable for the early detection of anomalies or unexpected patterns, allowing for more effective decision-making (Nguyen et al., 2020). Similar to normal machine learning tasks, anomaly detection can be approached in supervised, unsupervised or semi-supervised manner. Due to the sparseness of labels, in the last decade the problems are often approached using unsupervised methods (Pang & Van Den Hengel, 2020). However, Aggarwal (2017) argues in practise all readily accessible labeled data should be leveraged as much as possible. Semi-supervised detection methods do this by learning an expressive representation of normal behaviour training exclusively on normal labeled data (Pang & Van Den Hengel, 2020).

Anomaly detection is a unique problem with distinct problem complexities compared to the majority of machine learning tasks (Pang & Van Den Hengel, 2020). Anomalies are associated with many unknowns which remain unknown until they actually occur. These unknowns are related to abrupt behaviours, data structures and distributions. Anomalies also often show abnormal characteristics in a low-dimensional space hidden in a high-dimensional space, making it challenging to identify these. Moreover, the anomalies often depend on each other by a temporal relationship. Anomalies are often heterogeneous and irregular. Consequently, one class may have completely different characteristics from another anomaly. Due to the irregularity an anomaly is often rare and therefore severe class imbalance exists. As a result of these unique characteristics, obtaining a high detection recall rate, while reducing the false positives is the main challenge of each anomaly detection problem (Pang & Van Den Hengel, 2020). Literature distinguishes anomalies in three different types which are shown in FIG. 3 (Pang & Van Den Hengel, 2020; Chalapathy & Chawla, 2019). The majority of the anomaly detection literature focuses on this type of anomalies. Point anomalies represent an irregularity that happens randomly and may have no particular interpretation. Literature states methods for detecting point anomalies mainly rely on a single data sources and cannot be used for contextual and collective anomalies (Pang & Van Den Hengel, 2020).

Similar to point anomalies, contextual anomalies are also individual irregularities, but take place over time (Song et al. 2007). A collection of individual points is known as collective anomalies, where the individual members of the collective anomaly may not be anomalies (Chalapathy & Chawla, 2019; Pang & Van Den Hengel, 2020). During this literature study, detecting anomalous chocolate production patterns over time is the main task of the anomaly detection problem. Detecting anomalous patterns allows for a more effective decision making (Nguyen et al., 2020). Time series can be classified in univariate and multivariate time series. With univariate time series, only one features varies over time, whereas for multivariate time series multiple features change over time. Consequently, a chocolate batch is considered a multivariate time series sequence, for which the whole sequence is either classified as normal or anomalous. Contextual anomalies are considered as the main anomaly type due to the time factor and considering each chocolate batch as individual sequence. Detecting anomalies in time series also generates additional challenges because the pattern of the anomaly is often unknown and time series are usually non-stationary, non-linear and dynamically evolving. The performance of the algorithms is also affected by possible noise in the input data and the length of the time series increases computational complexity (Chalapathy & Chawla, 2019). Researchers often evaluate anomaly detection methods on its precision, recall and F1-score. Precision indicates how accurate the model is. It indicates out of positive predicted, how many of them are actual positive. Recall indicates the proportion of identified positives out of all actual positives. F1 score gives a measurement for the quality of a classifier by calculating a weighted fraction of recall and precision.

2.3.1 Anomaly Detection with Support Vector Machines

Conventional anomaly detection methods often use data mining, machine learning, computer vision and statistics (Pang & Van Den Hengel, 2020). Many researchers investigated the use of support vector machines to detect anomalies in time series (Wu et al., 2020). Early in (2005), Ribeiro compared different SVM classifiers for fault detection in a plastic injection molding process. Support vector machines are applied to monitor in-process data as a means of indicating product quality and enable quick responses to unexpected process disturbances. The SVMs require the data to be converted Into features. Dey, Prakash Rana, and Dudley (2018) applied SVMs to detect faults in building sensor data. Sensor data is often unstructured and unlabelled, which requires pre-processing in order to enhance machine learning models. Semi-supervised methods are proposed due to the data complexity and limited availability of labeled data. First train a supervised multi-class support vector machine algorithm for automated fault detection and diagnosis. Afterwards, they test the model on unlabelled data and validate the results using paired t-test. The paired t-test provides understanding in the correlation between historical labeled and predicted unlabelled data. Chen et al. (2020) also proposes to use multi-class support vector machines for control chart recognition. Their approach automatically extracts thirteen shape features and eight statistical features of control charts. The most representative feature set is used to train a multi class support vector machine algorithm which is successfully identifies anomalous control charts. Experimental analysis showed one against one support vector machines combined with majority voting yields highest classification accuracy. Additionally, SVMs can be applied to select the best performing model. Selecting a function support vector model to detect sparse defects within the process industry depends on a trade-off between three competing attributes: prediction as the generalization ability, separability distance between classes and complexity (Escobar & Morales-Menendez, 2019). An SVM can be used to separate the best performing model by mapping these attributes into a 3D space.

However, SVM seems to be sensitive to missing values and it only considers the characteristics of the current time point, rather than the time dependence of the time series feature (Wu et al., 2020). Another disadvantage is that traditional learning methods often require carefully engineered input features which in turn requires extensive domain knowledge. Contrary, deep learning automatically derive hierarchical hidden representations of raw input data (Pang & Van Den Hengel, 2020). Therefore, multiple studies argue deep anomaly detection are more suitable methods for time series anomaly detection compared to traditional machine learning methods. Deep learning has a lot of potential in situations where relevant input features are hard to define due to a lack of domain knowledge (Chalapathy & Chawla, 2019; Kieu, Yang, & Jensen, 2018).

2.3.2 Anomaly Detection using Deep Learning

Many researchers applied different architectures of recurrent neural networks for anomaly detection with multivariate time series data. An overview of recurrent neural network architectures to detect anomalies is given below:

### 2.3.2.1 Supervised Anomaly Detection

Nucci, Cui, Garrett, Singh, and Croley (2018) developed a real-time multivariate anomaly detection system for internet providers. Their system utilizes a four layer LSTM network to learn the normal behaviour and classify anomalies. Once the system classifies an anomaly an alert which is inspected by domain experts is created. The LSTM classification network is automatically re-calibrated using the judgements of domain experts. Over time their models become more precise in the categorization of the anomalies, translating into a higher operational efficiency. Unfortunately, their classification model requires many labeled instances of both normal and anomalous sequences. Hundman, Constantinou, Laporte, Colwell, and Soderstrom (2018) utilize LSTMs to detect anomalies in multivariate spacecraft telemetry data. Single LSTM models are created for each channel to predict the next time step channel value ahead. Utilizing single models for each channel facilitates traceability. High prediction performance is obtained by training the network using expert-labeled satellite data. Additionally, the authors propose an unsupervised and non parametric anomaly threshold approach using the mean and standard deviation of the error vectors. The anomaly threshold approach addresses diversity, non-stationary and noise issues associated with anomaly detection methods. At each time step and for each channel the prediction error is calculated and appended to a vector. Exponentially-weighted average is used to smooth and damp the error vectors. A threshold is used to evaluate whether values are considered as anomalies. Although, this study uses multivariate time series data, their prediction model only utilizes univariate time series and does not consider the interdependence of features. Nolle, Seeliger, and Mühlhäuser (2018) propose a recurrent neural network, trained to predict the name of the next event and its attributes. Their model focuses on multivariate anomaly detection in discrete sequences of events and is capable of detecting both point and contextual anomalies. However, the model predicts the next discrete events and is thus not applicable for conching, where the order of events is assumed to be constant.

### 2.3.2.2 Autoencoders

Many other studies investigate the use autoencoders to detect anomalies within various different applications. An and Cho (2015) describe the traditional autoencoder-based anomaly detection approach as a deviation-based anomaly detection method with semi-supervised learning. Autoencoder detection algorithms are typically trained exclusively on normal data. The anomaly score is determined by the reconstruction error, and samples with large reconstruction errors are predicted as anomalies.

An autoencoder is a neural network which learns a compressed representation of an input (Pang & Van Den Hengel, 2020). Training an autoencoder is performed in an unsupervised learning manner and is typically performed to recreate the input. Reconstructing the input is purposely challenged by restricting the architecture to a bottleneck in the middle of model. The heuristic for using autoencoders in anomaly detection, is that the learned feature representations are enforced to learn important regularities of the normal data to minimize the reconstruction error. It is assumed anomalies are difficult to reconstruct from these learned normal feature representation and thus have large reconstruction errors. Pan and Yang (2009) state advantages of using data reconstruction methods include the straight forward idea of autoencoders and its generic application to different types of data. However, the learned feature representations can be biased by infrequent regularities and the presence of outliers or anomalies in the training data. In addition, the objective function during training the autoencoder is focused for dimensionality reduction rather than anomaly detection. As a result the representations are a generic summarization of the underlying regularities, which are not optimized for anomaly detection.

Malhotra et al. (2016) propose to use an LSTM-based autoencoder to learn to reconstruct normal univariate time series behaviour of three publicly available data sets. After learning normal behaviour, the reconstruction error is used to detect anomalous time series within power demand, space shuttle and electrocardiogramata. Their experiments show the model is able to detect both anomalies from short time-series as well as long time-series. In case of a multivariate time series data set, the authors first reduce the multivariate time series to univariate using the first principal component of PCA. Similar, Assendorp (2017) developed multiple LSTM-based autoencoder models for anomaly detection in washing cycles using multivariate sensor data. In their first experiment and based on Malhotra et al. (2016), all sensor channels are reduced to the first principal component using PCA. The first principal component is reconstructed using an LSTM-based autoencoder. Their second experiments reconstruct the full sensor channels using an LSTM-based autoencoder. Results show deeper encoder and decoder network as well as bidirectional encoders reduce the reconstruction loss of normal sequences. In another experiment, Assendorp (2017) trained Generative Adversial autoencoders to learn a generative model on a specific data distribution. A major advantage of a GAN model includes the possibility to generate normal sequences. However, experiments showed the GAN network seemed not capable of detecting anomalies. Additionally, GANs might be difficult to use for general anomaly detection because they require several tricks for training (Chintala, Denton, Arjovsky, & Mathieu, 2016). Kieu et al. (2018) propose a framework for detecting dangerous driving behaviour and hazardous road locations using time series data. First, a method for enrichment of the feature spaces of raw time series is proposed. Sliding windows of the raw time series data are enriched with statistical features such as mean, minimum, maximum and standard deviation. Then, the authors examine 2D Convolutional autoencoder and LSTM autoencoder and one-class Support Vector Machines to detect outliers. It was found enriched LSTM autoencoders achieves best prediction performance, which shows deep neural networks are more accurate than traditional methods.

Even though an LSTM unit performs better compared to a classic RNN network, classical LSTM autoencoders still suffer from long sequences. In a classical sequence-to-sequence auto-encoder model, the encoder encodes the entire sequence in its hidden state at the last time step. This hidden state is then fed into a decoder to predict the input sequence. In many sequence-to-sequence learning problems, it was found that the encoded state was not enough for the decoder to predict the outputs (Dai & Le, 2015). Kundu, Sahu, Serpedin, and Davis (2020) state incorporating an attention mechanism with the autoencoder can solve this problem.

2.3.2.3 Autoencoders with Attention Mechanism

Attention based autoencoders utilize every hidden state from each encoder node at every time step and then reconstruct after deciding which one is more informative. It allows one to find the optimal weight of every encoder output for computing the decoder inputs at a given time-step. Both, Kundu et al. (2020) and Pereira and Silveira (2019) investigated incorporating attention mechanism with autoencoders for detecting anomalies. Kundu et al. (2020) demonstrate how an LSTM autoencoder with an attention mechanism is better at detecting false data injections compared to normal autoencoders or unsupervised one-class SVMs. The authors detect attacks in a transmission system with electric power data. Anomalous data is detected due to high reconstruction errors and through selecting a proper threshold. Similar, Pereira and Silveira (2019) propose a variational self-attention mechanism to improve the performance of the encoding and decoding process. A major advantage of incorporating attention, is that it facilitates more interpretability compared to normal autoencoders (Pereira & Silveira, 2019). Their approach is demonstrated to detect anomalous behaviour in solar energy systems, which can trigger alerts and enable maintenance operations.

### 2.3.2.4 Variational Autoencoders

Normal autoencoders, as described in Section 6.4.3, learn to encode input sequences to a low-dimensional latent space, but variational autoencoders are more complex. A variational autoencoder is a probabilistic model that combines the autoencoder framework with Bayesian inference. The theory behind VAE is that numerous complex data distributions may be modeled using a smaller set of latent variables with easier-to-model probability density distributions. The goal of VAE is to find a low-dimensional representation of the input data using latent variables (Guo et al., 2018). As a result various researchers investigated its application for anomaly detection.

Suh, Chae, Kang, and Choi (2016) introduced an enhanced VAE for multidimensional time series data to take the temporal dependencies in fictive data into account and demonstrated its good accuracy compared to conventional algorithms for time-series monitoring. (Ikeda, Tajiri, Nakano, Watanabe, & Ishibashi, 2019) propose to utilize a VAE to detect the presence of medical arrhythmia in cardiac rhythms or detect network attacks. The VAE estimates the dimensions which contribute to the detected anomaly. The authors state the probabilistic modeling can also be used for giving interpretations. Traditional variational autoencoders generally assume a single-modal Gaussian Distribution. Due to the intrinsic multi-modality in time series data, traditional AEs can fail to learn the complex data distributions and hence fail in detecting anomalies (Guo et al., 2018). Therefore, Guo et al. (2018) propose a variational autoencoder with Gated Recurrent Unit cells system to detect anomalies. Their approach is tested in two different settings with temperature recordings in a lab and Yahoo's network traffic data. Gated Recurrent unit cells discover the correlations among time series inside their variational autoencoder system. Similar, D. Park, Hoshi, and Kemp (2018) introduce a long short-term memory-based variational autoencoder to learn utilizes multivariate time series signals and reconstructs their expected distribution. The model detects an anomaly in sensor data generated by robot executions, when the log-likelihood of the current observation given the expected distribution is lower than certain threshold. In addition, the authors introduce a state-based threshold to increase sensitivity and lower the false alarms. Their variational autoencoding using LSTM units and state-based threshold method seems effective in detecting anomalies without significant feature engineering effort. Similar, the earlier described Pereira and Silveira (2019) propose a variational autoencoder, enhanced with an attention model, to detect anomalies in solar energy time series.

### 2.3.2.5 Deep Hybrid Anomaly Detection Models

Once the prediction and its prediction error are calculated, often a threshold is set which is used to determine whether a given time step is considered as an anomaly. At this stage, an appropriate anomaly threshold is sometimes learned with supervised methods that use labelled examples (Hundman et al., 2018). Utilizing supervised methods after using an autoencoder is considered as a hybrid model and often combined with support vector machines. In their paper, Nguyen et al. (2020), suggest to use one-class support vector machine (OCSVM) algorithm to separate anomalies from normal samples based on the output of an LSTM autoencoder network. The deep hybrid model is evaluated for anomaly detection using real fashion retail data. For each sliding window the model computes the reconstruction error vector which is used to detect an anomaly. Detecting anomalies based on the error vectors normally assumes these vectors follow a Gaussian distribution (Malhotra et al., 2016), which is often untrue. Nguyen et al. (2020) propose to overcome this issue by using unsupervised machine learning algorithms that do not require any assumption of data. OCSVM could draw a hyper-plane which separates anomalous observations from normal observations. On the other hand if labels are available, it is also possible to combine the output of autoencoders with supervised algorithms. Fu, Luo, Zhong, and Lin (2019) demonstrate how convolutions autoencoders and SVM can be combined to detect aircraft engine faults. Convolutional autoencoders are known for its good performance in many high-dimensional and complex pattern recognition problems. Fu et al. (2019) suggest to utilize multiple convolutional autoencoders for different feature groups. For each group, convolutional feature mapping and pooling is applied to extract new features. All new features are combined into a new feature vector which is then fed to an SVM model. The supervised SVM accurately identifies anomalies using this new feature vector. Similar, approach is suggested by Ghrib, Jaziri, and Romdhane (2020). The authors proposed to combine the latent representation of the LSTM autoencoder with a SVM to detect fraudulent bank transactions. The proposed model inherits the autoencoders ability of learning efficient representations by only utilizing the encoder part of a pretrained autoencoder.

### 2.4 Discussion

The conducted literature review discussed three main topics: the existing methods for controlling food mixing processes, machine learning applications in the food industry and anomaly detection. First, literature states, from a business perspective, techniques in the food processing industry should be as straightforward, efficient, and non-invasive. In large scaled production plants with multiple machines, techniques such as phenomenological models and advanced sensors are not applicable. Secondly, machine learning may be a novel technology that can be used to facilitate the design of quality during the actual manufacturing process. Moreover, it can be customized to specific task and does not require the challenging development of first-principle models. Several researchers have successfully used supervised learning techniques in a variety of food-related applications. Quality control and detection are found to be common objectives of such learning applications within the food industry. To the best of the inventor's knowledge, Gunaratne et al. (2019) and Benković et al. (2015) are the only work to predict chocolate properties using machine learning. Both authors utilize neural networks, the first predicts properties during the production of liquid chocolate, whereas the latter predicts properties of chocolate powder samples. However, supervised learning demands a sufficient number of qualitatively labeled examples. Because qualitative labels are typically insufficient in large-scale industrial operations, semi-supervised learning techniques are recommended.

Finally, the traditional autoencoder-based anomaly detection approach is considered as semi-supervised learning. Anomaly detection detects samples which deviate from normal behaviour and shows great potential to improve the operational stability of industrial processes in various applications. Applications are diverse such as engine faults detection, fraud detection, medical domains, cloud, monitoring or network intrusions detection. Deep anomaly detection methods derive hierarchical hidden representations of raw input data and are considered to be best suitable for time-series detection. However, the availability of labels facilitates the possibility of hybrid anomaly detection models. Utilizing supervised methods after using an autoencoder is considered as a hybrid model and is often combined with support vector machines. This study extends current literature by exploring the use of various outputs of different autoencoders as input to other supervised learning models. It is believed, that applying semi-supervised deep hybrid anomaly detection methods during the production of chocolate is innovative and contributes both to the literature in controlling food mixing processes, as well as the anomaly detection literature.

### SUMMARY OF THE INVENTION

According to aspects of the invention, there are provided computer-implemented methods as defined in the independent claims. Advantageous features are set out in the subclaims.

According to one aspect, there is provided a computer-implemented method of predicting quality of a food product sample after a mixing process. The quality prediction is based on properties of the food product. For instance, the quality prediction is based on properties of the food product itself and/or properties/parameters of the mixing process. The mixing process may be part of a manufacturing process, performed on a manufacturing line.

The method involves building a (deep) hybrid model. The hybrid artificial intelligence model comprises an autoencoder machine learning model and a supervised machine learning model. The process of building a hybrid model includes, firstly, training an autoencoder. An autoencoder typically comprises an encoder network and a decoder network. This autoencoder training is performed in an unsupervised learning step (that is, learning using unlabelled datasets). This unsupervised learning step uses historical process data of food product samples. As an example, the method may use a long short-term memory (LSTM) network autoencoder; one benefit of using an LSTM-autoencoder is that it eliminates the need for preparing hand-crafted features and thus facilitates the possibility to use raw data with minimal pre-processing. In this way, the autoencoder may be used as a feature extractor.

This process of building a hybrid model includes, secondly, training a supervised model in a supervised learning step (that is, learning using a labelled dataset). This supervised learning step uses the output of the (trained) autoencoder. For instance, the supervised learning step may use the error vector over time and the hidden space (or latent space) generated by the autoencoder.

The method then includes predicting the quality of the food product. This prediction is performed by inputting process data of current samples into the (trained) hybrid model. The hybrid model then classifies the current samples. In this way, the hybrid model involves the autoencoder feeding the supervised, anomaly detection algorithm. This classification allows detection of anomalous behaviour of the mixing process. For example, the classification may be “normal” or “anomalous”, or may be a graded classification.

Optionally, the method of predicting quality of a food product sample may use sensors to capture online and/or inline process data from a food manufacturing line. Online methods automatically take samples (from the manufacturing line) to be analysed without stopping, whereas inline methods directly measure the process stream without sample removal. The process data captured by the sensors may be used as historical process data, for training purposes. Additionally or alternatively, the process data captured by the sensors may be used as current process data, for prediction purposes. In either case, the use of sensors allows for automated data collection, removing the possible need for manual sampling.

Optionally, the process data may include raw material quantity data. Further, the process data may include mixing engine characteristics. For instance, the mixing engine temperature, rotation speed, power, etc. may be used.

Optionally, the process data may be truncated at a predetermined time. As the process data may be unlabelled, or labels are only known for the whole data sequence (e.g., average speed of mixing process), a variation in length of process data sequences may be difficult to handle (e.g., using a sliding window approach). Truncating sequences at a particular, predetermined time enables early anomaly detection. In addition, the truncation ensures that no data sequence needs to be padded (e.g., with zeros) to ensure identical length of data sequence.

Optionally, the method of predicting quality of a food product sample may comprise alerting an operator of an expected anomalous batch of food product if one or more samples is classified as anomalous. For instance, the hybrid model may be used as an alarming method in case a faulty batch occurs. This enables maintenance operations to be performed only when required (removing the need for unnecessary halting of a production process).

Optionally, the autoencoder may include an attention mechanism. The attention mechanism may be additive, multiplicative, or any other variation thereof. An attention mechanism assigns weights to every input sequence element and its contribution to every output sequence element, and enables encoding of past measurements with required importance to the present measurement. This helps to look at all hidden states from the encoder sequences within the autoencoder. That is, the hybrid model is able devote more focus to the small, but important, parts of the process data.

Optionally, the supervised learning model may be a random forest binary classification model. This random forest model may add randomness and generate decorrelated decision trees. Advantageously, the hybrid model with a random forest is not prone to over-fitting, has good tolerance against outliers and noise, is not sensitive to multi-collinearity in the data, and can handle data both in discrete and continuous form.

Optionally, the autoencoder of the hybrid model may be trained in a semi-supervised manner. Firstly, the autoencoder may be trained in an unsupervised manner, on purely normal samples (i.e., process data that does not include or relate to any anomalous samples). The autoencoder may then be trained a validation set of normal and anomalous samples (the anomalous set). The anomalous validation set may be used for supervised parameter tuning by setting an error threshold. Using a semi-supervised training method, the autoencoder is able to accurately distinguish normal samples from anomalous samples.

Optionally, the food product may be a confectionary product. For instance, the food product may be chocolate or caramel or cookie dough. Further, when the confectionary product is chocolate, the mixing process may be conching. In conching, a surface scraping mixer and agitator (conche) distribute cocoa butter within chocolate. When the method of quality prediction is applied to a conching process, the method enables accurate prediction of sample quality in an otherwise complex and non-linear process.

Optionally, the properties used in the determination of whether samples are labelled normal or anomalous are any or all of: yield stress of the food product being mixed (e.g., measured in pascals, Pa); viscosity of the food product being mixed (e.g., measured in pascal seconds, Pas); fat content of the food product being mixed (e.g., measured in percent of total mass/weight of product); and moisture (e.g., in percent of total mass/weight of product). These properties may be determined using in-line (or on-line) sensors after (or during) the mixing process. Preferably, a “normal sample” (i.e., non-anomalous) may be indicated/classified when the property (e.g., yield stress, viscosity, . . . ) is within a suitable, given (predetermined or flexibly calculated) range.

Optionally, the output of the autoencoder comprises a reconstruction error of the autoencoder. In this way, samples with large reconstruction errors may be predicted or classified as anomalies. In using autoencoders for anomaly detection, the learned feature representations may be forced to learn important regularities of the normal data to minimize the reconstruction error. It is assumed anomalies are difficult to reconstruct from these learned normal feature representation and thus have large reconstruction errors. Thus, reconstruction error provides a simple metric for sample anomaly.

Optionally, predicting the quality of the food product may comprise inputting process data of current samples to the autoencoder. The autoencoder may be configured to compress the input process data to a latent space, and to reconstruct the process data from the latent space. The prediction then involves generating a reconstruction error between the input process data and the reconstructed process data. The prediction then involves inputting the reconstruction error to the supervised model. The supervised model may be configured to process the reconstruction error according to supervised model parameters set during the supervised learning step. The prediction then involves obtaining, from the supervised model, an output. The output may comprise a predicted value of a measure, which indicates the quality of the food product.

Optionally, training the supervised model using the output of the autoencoder may comprises assembling a training data set comprising, for historical process data of food product samples, outputs from the autoencoder and labels corresponding to the outputs, Further the dataset may comprise values of a measure indicating quality of the food product. Training the supervised model then uses the assembled training data set to update trainable parameters of the supervised model.

Optionally, using the assembled training data set to update trainable parameters of the supervised model may comprise inputting the outputs from the autoencoder to the supervised model and obtaining-from the supervised model-outputs comprising a predicted value of a measure indicating quality of the food product. The method may then update trainable parameters of the supervised model so as to minimise a loss function based on a difference between the output of the supervised model and the labels of the training data set.

Embodiments of further aspects include a trained hybrid model, used to carry out a classification method as variously described herein (according to an embodiment). The module may be positioned in the neural network after an encoder module and before a pooling module, or in any other suitable position.

Embodiments of a still further aspect include a data processing apparatus comprising means for carrying out a method as variously described above.

Embodiments of another aspect include a computer program comprising instructions, which, when the program is executed by a computer, cause the computer to carry out the method of an embodiment. The computer program may be stored on a computer-readable medium. The computer-readable medium may be non-transitory.

Hence embodiments of another aspect include a non-transitory computer-readable (storage) medium comprising instructions, which, when the program is executed by a computer, cause the computer to carry out the method of an embodiment.

The invention may be implemented in digital electronic circuitry, or in computer hardware, firmware, software, or in combinations thereof. The invention may be implemented as a computer program or a computer program product, i.e., a computer program tangibly embodied in a non-transitory information carrier, e.g., in a machine-readable storage device or in a propagated signal, for execution by, or to control the operation of, one or more hardware modules. A computer program may be in the form of a stand-alone program, a computer program portion, or more than one computer program, and may be written in any form of programming language, including compiled or interpreted languages, and it may be deployed in any form, including as a stand-alone program or as a module, component, subroutine, or other unit suitable for use in a data processing environment.

The invention is described in terms of particular embodiments. Other embodiments are within the scope of the following claims. For example, the steps of the invention may be performed in a different order and still achieve desirable results.

### BRIEF DESCRIPTION OF THE DRAWINGS

Reference is made, by way of example only, to the accompanying drawings in which:

FIG. 1 is a flow chart of a method for predicting quality of a food product sample after a mixing process, based on properties of the food product, according to an embodiment;

FIG. 2 is a general framework of data analytics capabilities in a known manufacturing process;

FIG. 3 is a schematic diagram of known anomaly types;

FIG. 4 is a diagram of a problem solving cycle and a diagram of the CRISP-DM framework

FIG. 5 is a diagram of a conche machine;

FIG. 6 illustrates correlations between final measured chocolate properties;

FIG. 7 is a set of diagrams showing occurrences of chocolate viscosity measurements, illustrating the imbalanced distribution;

FIG. 8 is a set of diagrams showing occurrences of Occurrences first chocolate property measurement categorized by the different machines;

FIG. 9 is a set of diagrams showing occurrences of chocolate faults, illustrating the imbalanced distribution;

FIG. 10 is a distribution of the observed faults. The anomaly class is constructed using [Viscosity, Yield, Fat Content, Moisture]. 0 indicates the measured value is within the limits, whereas (−)1 indicates the measured value is above (below) the limit;

FIG. 11 is a diagram of example first principal component analysis over time categorized by the chocolate batch outcome;

FIG. 12 is a schematic diagram of construction of binary target label;

FIG. 12b is a diagram of applied pre-processing steps;

FIG. 13 is a schematic diagram illustrating truncating sequences with different lengths;

FIG. 14 is a schematic diagram illustrating generation of training, testing, and validation sets;

FIG. 15 is a schematic diagram of a recurrent neural network structure;

FIG. 16 is a schematic diagram of a LSTM unit;

FIG. 17 is a schematic diagram of an autoencoder structure;

FIG. 18 is a comparative diagram of normal vs attention based autoencoder architecture;

FIG. 19 is a schematic diagram of variational autoencoder architecture;

FIG. 20 is a schematic diagram of a final deep hybrid model according to an embodiment;

FIG. 21 is a set of confusion matrices for a supervised LSTM classifier benchmark model according to an embodiment;

FIG. 22 is a set of diagrams illustrating training and validation losses for different autoencoders, according to embodiments;

FIG. 23a is a set of attention maps for normal and anomalous cases in an example test set, where lighter colour indicates more attention is assigned to a certain time step;

FIG. 23b is a set of attention maps for normal and anomalous cases in an example test set, for a length of 240 minutes;

FIG. 23c is a set of attention maps for normal and anomalous cases in an example test set, for a length of 300 minutes;

FIG. 24a is a set of diagrams illustrating normal autoencoder validation set reconstruction loss distributions, where the anomalous samples are shown darker and the good samples lighter;

FIG. 24b is a set of diagrams illustrating attention autoencoder validation set reconstruction loss distributions;

FIG. 24c is a set of diagrams illustrating variational autoencoder validation set reconstruction loss distributions;

FIG. 25a is a precision recall curve and a threshold curve for a normal autoencoder, explaining graphically how the validation set may be used to determine a threshold;

FIG. 25b is a precision recall curve and a threshold curve for an attention autoencoder;

FIG. 25c is a precision recall curve and a threshold curve for a variational autoencoder;

FIG. 26 is a set of confusion matrices for final test performance for three different autoencoders according to embodiments;

FIG. 27 is a table showing average and standard deviation of the test performances when using different validation and test splits for autoencoders trained on different lengths, according to embodiments;

FIG. 28 is a table showing sensitivity analysis using different validation test splits according to embodiments;

FIG. 29 is a set of diagrams illustrating distribution of detected anomalies and undetected anomalies, where [viscosity, yield, fat content, moisture] denote the chocolate batch outcome used to construct the binary label;

FIG. 30 is a set of confusion matrices for LSTM Classifier Benchmark Model;

FIG. 30b is a set of diagrams illustrating training and validation loss;

FIG. 31 is a set of attention maps for normal and anomalous cases (based on control limits) in the test set, where lighter colour indicates more attention is assigned to a certain time step;

FIG. 32a is a set of diagrams illustrating the mean squared error of samples within the validation set generated by the normal autoencoder;

FIG. 32b is a set of diagrams illustrating the distribution of MSE reconstruction loss;

FIG. 32c is a set of diagrams illustrating precision and recall Curves which graphically show how thresholds are determined;

FIG. 33 is a table showing average and standard deviation of the test performances when using different validation and test splits, according to embodiments. For each model, the threshold is determined using the FB value on the validation set;

FIG. 34 is a table showing sensitivity analysis using different validation test Splits, test performance variability result;

FIG. 35 is a table showing a comparison of best thresholding model trained on different labels;

FIG. 36 is a table showing a further comparison of best thresholding model trained on different labels;

FIG. 37 is a diagram illustrating test performance for best performing deep hybrid detection model, according to an embodiment;

FIG. 38 is a set of specification limits sample force plots;

FIG. 39 is a diagram illustrating control limits for an autoencoder with RandomForest—Shap Values, according to an embodiment;

FIG. 40 is a set of diagrams illustrating classification results, distributed per machine;

FIG. 41 is a set of diagrams illustrating types of misclassifications based on the chocolate batch outcome;

FIG. 42 is a diagram of suitable hardware for implementation of invention embodiments.

### DETAILED DESCRIPTION

Controlling food processes is difficult because disturbances are easily propagated throughout the process, which affect the quality of the final product. One of the main objectives of food processing operations is thus to limit the variability such that consistent objective quality is obtained. As an example, this document concerns chocolate production but the skilled reader will appreciate that the techniques disclosed herein are applicable to production of other food products.

Chocolate production includes non-linear characteristics, such as crystallization, which makes online monitoring and process control additionally challenging. As a consequence, chocolate manufacturers require an efficient and reliable method for product and quality control. In recent years, digitization gave rise to large amounts of data and analyzing this data could enhance process understanding and efficiency. The motivation for this study is to investigate the potential of machine learning techniques in order to detect an incorrect behaving chocolate batch which can enhance the chocolate production control.

Chocolate confectionery production typically consists of multiple phases, which starts with the chocolate production known as conching. The chocolate production step is examined during this study because for this step the most data is easily available, though—again—the skilled reader will appreciate the techniques disclosed herein are applicable to production of other food products and to other production processes. Moreover, it is the production phase which is seen as the internal black box where little knowledge is available. Conching evenly distributes cacao-butter within chocolate to obtain a homogeneous mixture. Any variability in the semi-finished chocolate properties cause problems downstream the manufacturing lines. Mars current control practice is reactive because it measures the chocolate properties yield, viscosity, fat content and moisture using at-line sensors at the end of the production cycle. Moreover, an experienced operator can detect an incorrect process by manually monitoring the process. However, in such case the correctness of the detection is always unknown. Mars can thus only adapt the process with certainty when the properties are known, which can further delay the production process. A batch process is considered to be in control if all four properties are within control limits.

The goal of this research is to increase the overall process control by utilizing (a combination of) data-driven methods. These data-driven methods can be used to detect incorrect process behaviour and possibly investigate relations between production log data and chocolate properties. The data-driven approach is chosen because Mars stores a large amount of data at different systems without using this data to its full potential. It is performed in an online fashion, by using online process data which can be used to enhance quality control. Process log data related to raw material usage and engine characteristics over time serves as input for a deep hybrid machine learning model which tries to predict whether the current production cycle is in control. Current literature proposes manual sampling or advanced online sensors to feed scientific models or neural networks to predict chocolate properties. Another neural network required the full power curve of the main engine to predict the final viscosity. However, these methods are from a business perspective not practical for Mars because these require an accurate prediction early in the process. Moreover, advanced online sensor technology may not be suitable for large-scaled factories due to the high cost, while manual sampling limits real-time monitoring. This study extends the current literature by using the process log data to make an early prediction.

Data preparation resulted in a data set consisting of 1917 chocolate sequences with 21 process features which vary over time. All features are related to the usage of raw materials during the process or include actual conche characteristics. For each raw material, both PLC control indicators and a numeric feature indicate whenever and how much material are used. Further, conche characteristics regarding temperature, revolutions per minute and power are used. Each sequence corresponds to one chocolate production cycle which is eventually measured on four properties during the after mixing phase. Data exploration highlighted the difficulty of the faced problem as it turned out the dataset is highly imbalanced. The low availability of anomalous sequences limits classification possibilities, as such it is chosen to classify sequences as correct and incorrect. These two groups showed very little differences in the smoothed average of a single feature or the first principal component over time, making it even more complex. It was chosen to tackle the imbalanced nature of the target class by applying anomaly detection methods. Anomaly detection methods learn the ideal representations through autoencoders and learn these from the complete majority set. Anomaly detection is often performed in an unsupervised manner because labels are unknown. This research extends current anomaly detection literature by combining the output of various unsupervised autoencoders with supervised learning models into a deep hybrid detection model. As a result, different autoencoders which detect an anomaly by setting a reconstruction error threshold and a deep hybrid classification models which use autoencoders as feature engineering are compared. The advantage of the latter includes that it uses both the good sequences and incorrect samples, such that minimal information is lost during training. Training the autoencoders on shorter sequences showed better anomaly detection capabilities, because the performance decreased as the length of the sequence increased, indicating that the autoencoders which are trained exclusively on good behaviour learn more noise with longer sequences.

A deep hybrid approach which combines an unsupervised attention-based autoencoder, trained on “within control limit” chocolate batches, with a supervised Random Forest binary classification model exhibits the best performance. According to the test set's sensitivity analysis, the model can robustly notify an operator with nearly 70% precision and detect around 40% of all problematic out of control batches. Implementing such a model could increase the efficiency of the process and reduce operator workload. Currently, Mars relies on the operators to detect an incorrect chocolate batch on a specific conche in an early phase, which is additionally uncertain. Each operator must monitor multiple conches from a milling group, the anomaly detection model could emphasize the batch which is expected to become faulty with high certainty. . . . Moreover, both the attention mechanism and the supervised learning method enabled model interpretation. SHAP values can be utilized to interpret predictions from both a model and sample perspective, while the attention mechanism can be used to visualize essential minutes for reconstructing the time series of a sample. Both SHAP and attention weight evaluations accentuated the importance of the duration of the filling phase and therefore the main recommendation considers minimizing any disturbances within this period.

To conclude, this research investigated how Mars' current available data could be utilized to enhance the chocolate production control. This research showed the capabilities of neural networks to learn processing behaviour.

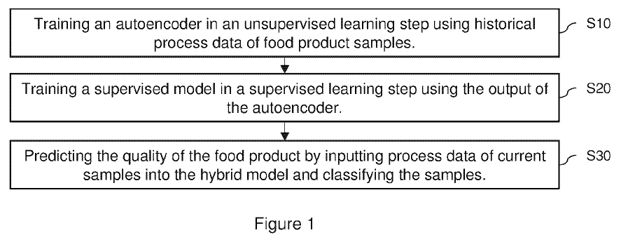

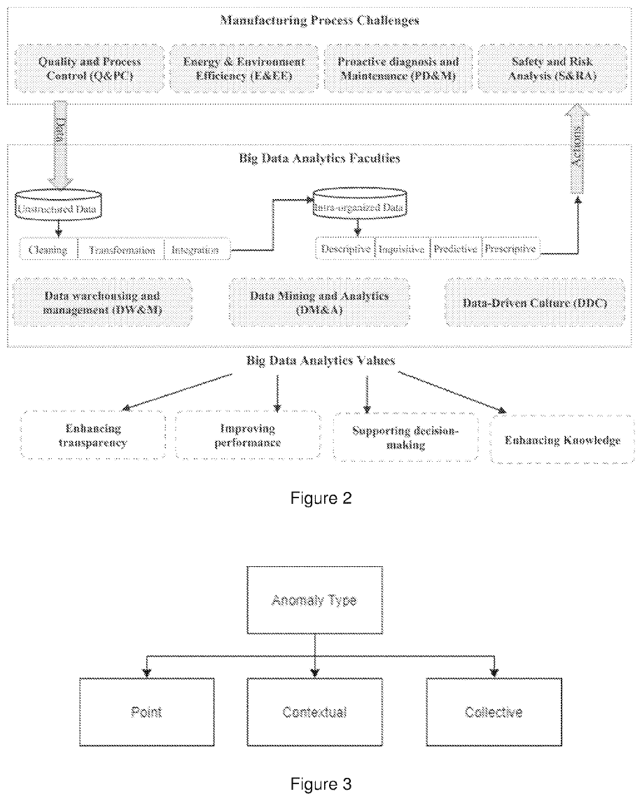

FIG. 1 is a block diagram, depicting a method for predicting quality of a food product sample after a mixing process, based on properties of the food product, according to an embodiment. S10 and S20 see the building/generation of a hybrid model: S10 trains an autoencoder in an unsupervised learning step using historical process data of food product samples and S20 trains a supervised model in a supervised learning step using the output of the autoencoder. S30 then predicts the quality of the food product by inputting process data of current samples into the hybrid model and classifying the samples.

### 3 Methodology

In order to effectively research and solve a specific problem, the research has to be performed systematically (van Aken, Berends, & Van der Bij, 2012). This section therefore introduces the research methodology which is applied thorough the research.

### 3.1 Problem Solving Cycle

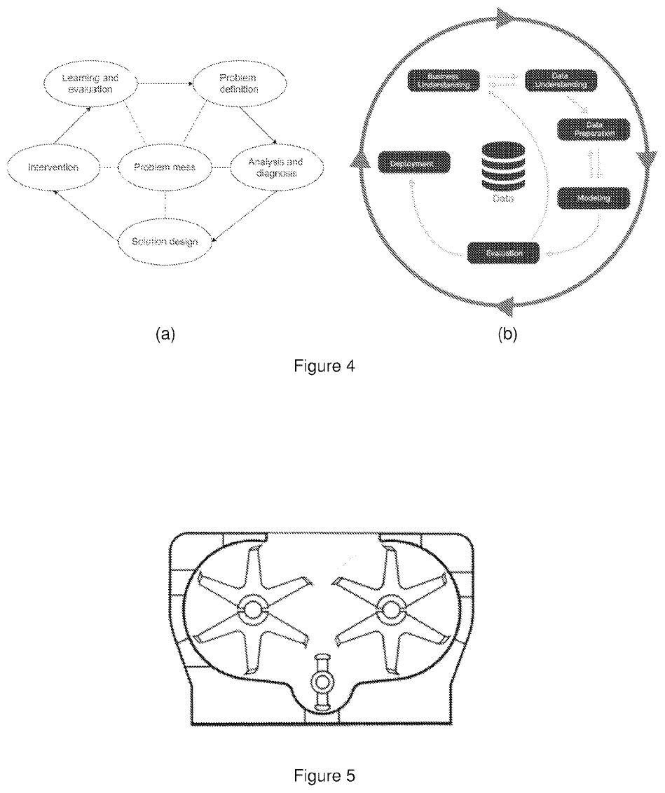

The research adheres to the problem-solving cycle, which is a design-oriented and theory-based process for creating solutions to field problems (van Aken et al., 2012). When a business problem emerges within a company, the problem solving cycle technique comes in useful. Business challenges are frequently a collection of interrelated problems, also referred to as a problem mess. In order to formulate a clear business problem, during the preliminary research proposal phase, this “problem mess” has been identified and structured. Structuring and identifying is the first step of the problem solving cycle and resulted in a problem definition, which is summarized in Chapter 1. The structuring step is followed by four more steps, which eventually result in a problem solution, which is implemented and evaluated (as shown in FIG. 4). During this research the following two steps analysis and diagnosis of the problem and solution design are executed. In order to further structure the research project, these two steps are approached using the CRISP-DM methodology, which is further explained in the next section. The remaining two phases (intervention and learning and evaluation) will be shortly addressed and will essentially serve as a preliminary assessment of the solution design. Due to the project's imposed time limits, completing the problem-solving cycle in its entirety is not feasible.

### 3.2 CRISP-DM

In order to approach this project in a structured manner, and systematically work towards the project goal, the Cross Industry Standard Process for Data Mining (CRISP-DM) is used. CRISP-DM is the most widespread methodology used for knowledge discovery. The methodology breaks down the life cycle of a data science project into six phases, as depicted in FIG. 4. The project is initiated by first gaining business understanding. Business understanding focuses on the project objectives and requirements and converting this knowledge into a project. The consecutive data understanding phase starts with an initial data collection and activities in order to get familiar with the data. During the data understanding phase, the quality of the data is assessed, first insights are generated and interesting subsets are obtained. The data preparation phase covers all activities to construct useful data sets serving as input for machine learning algorithms. During the modeling phase, different techniques are discovered, selected and applied. Afterwards these models are then tested in the evaluation phase and the best evaluated model is deployed during the deployment phase (Wirth & Hipp, 2000). The sequence of the phases is not rigid as moving back and forth between different phases is often required.

These phases overlap with the phases in the problem solving cycle: business understanding and data understanding are covered by the analysis and diagnosis phase while data preparation, modelling and evaluation are captured in solution design. The last step of the CRISP-DM framework, deployment, is closely related to the intervention step in the problem-solving cycle and is only partially addressed in this research project. The main focus of this project will be creating a data-driven learning model which predicts the quality of chocolate batches. The project will serve as a proof of concept for Mars Chocolate manufacturing environment. As a result, the deployment phase of the CRISP-DM model is less relevant during this study. However, all other phases of the methodology provide a solid structure to successfully perform a data-driven research within Mars.

Large Part of the business understanding phase has been addressed in Chapter 1. There the business problem has been formulated. Another part of the business understanding phase, concerns assessing the current business situation and processes. This assessment is performed in Chapter 4. The Data understanding is performed in Chapter 5 and involves taking a look at the available data and quality of the data. The business understanding and data understanding contribute to the final selection of data sources that are accessible for this research project. The data preparation phases are performed accordingly. During the modeling phase the final chocolate process anomaly detection model is constructed. Therefore, both phases are shown in Chapter 6, which answer the fourth research question. This chapter first describes the set of features selected for the modeling, how they are pre-processed and how the final data sets are constructed. Afterwards, the actual modeling approach is explained by first elaborating on Recurrent Neural Network Units and the used Long Short-Term Memory Units. LSTM units are applied within different autoencoder types, for which the output is finally used in supervised learning algorithms. Finally the model evaluation is performed in Chapters 7 and 8. In the conclusion of this research, the deployment phase will be briefly touched upon by providing implementation recommendations.

### 4 Application Background and Data Sources

This chapter belongs to the business understanding of the CRISP-DM methodology. As this research is conducted at Mars, it is important the develop the problem statement within the company. This chapter first explains the actual chocolate production process and the measured properties. It describes the current practice for monitoring by explaining how certain raw materials are used to influence the process and further explains current unexplored uncertainties supporting the problem statement. The chapter finalizes with identifying the important data sources available for this problem at hand.

### 4.1 Milling Groups

In Veghel, in total of 21 conche machines are aligned in different milling groups. In total there are four different milling groups and each group is able to produce different chocolate recipes. The chocolate powder determines the type of chocolate recipe and is either produced on Type A or Type B. The first and second conche groups are capable of producing the two types of chocolate Recipe A and Recipe B, whereas the third conche group is dedicated to producing the main chocolate recipe. A totally different type of chocolate is produced by the fourth conche group. This conche group produces multiple types of Type B chocolates.

Conches dedicated to producing Type A chocolate differ from conches that produce Type B chocolate. Each conche has its own parameter which regulate the actual process. In general, the parameters among conches within a conche group are quite similar. All conches have an engine with a similar electrical power, and thus have similar settings. Unfortunately, there is no such a system which logs the changes in conche parameters. For this research it is chosen to focus on conches of Type A 3 which produce exclusively Recipe A chocolate. Recipe A being the most produced recipe at Mars combined with the largest production group contributes to obtaining the largest possible sample size. Further, it is chosen not to include conches from Type A 1 and Type A 2 because these have different settings compared to Type A 3. Therefore, for the remainder of this research it is chosen to focus on Recipe A.

### 4.2 Production of Chocolate

As explained, this study focuses chocolate production at Mars. Chocolate manufacturing is known as a very complex process which requires a combination of several ingredients and technological operations to achieve the desired quality (Afoakwa, 2016). Chocolate is produced on the conche machine, which is illustrated in FIG. 5. Conching is an expensive process which requires high levels of energy consumption and limits capacities of the equipment (González et al., 2020). Conching is a process which evenly distributes cacao-butter within chocolate. As a result, a homogeneous mixture is obtained. In Veghel, different chocolate recipes are further used to produce different brands of chocolate bars. Besides producing the bars in Veghel, the semi-finished chocolate is further distributed to the other manufacturing locations of Mars around the world.

At Mars, conching is an automated batching process that tries to ensure the correct composition of chocolate considering the fat content, yield strength, viscosity and moisture. The process consists of different phases.

### 4.3 Properties of Chocolate

There are three different types of properties of chocolate, which include surface, sensory and physical quality. The surface quality is defined by the colour, shine and bloom, whereas taste and smell define the sensory quality. At Mars during the production of chocolate, the operators only steer towards the physical properties. It is assumed that these are most important and the bars are eventually tested on their surface and sensory quality at a later stage. Rheology, particle size, moisture, fat content and hardness define the physical state of chocolate. Rheology is a branch of physics that deals with the deformation and flow of materials. Within Mars rheology is conceptualized by using viscosity and yield stress. Viscosity is defined as the energy or force to keep the chocolate in motion, while yield stress is conceptualized as the minimum amount of energy to initiate fluid flow. Precise knowledge of the rheological properties of food is essential for the product development, sensory evaluation and design, quality control, and evaluation of the process equipment (Kumbár, Nedomová, Ondrušíková, & Polcar, 2018).

During the after-mixing phase for each batch a sample is taken. An operator then lubricates the sample on a little plate and places the plate in a rotational Rheometer (Anton Paar, Graz, Austria). This machine automatically determines the rheology of the sample and registers it in Sycon SubGroups Reports. Each chocolate sample is analyzed in rotational mode to determine the chocolate flow curve. The flow curve is obtained by the machine automatically rotating at a pre-set of shear rates and measuring the corresponding shear stress (Anton Paar, n.d.). Afterwards using Newton's Law the corresponding viscosity and yield stress can be calculated. Newton's Law defines the viscosity as the shear stress divided by the shear rate (Equation 1) (Mezger, 2011).

$$η = \frac{τ}{γ}$$

Where Viscosity is denoted by n, Shear stress and shear rate are represented by respectively t and y. Yield Stress can also be determined using the chocolate flow curves. The curve is measured using a linear increase of the shear rate. In order to determine the yield stress, the Anton Paar machine automatically fits the chocolate rheological flow using the Herschel-Bulkley model (Equation 2).

$$τ = {τ}_{HB} + c * {γ}^{p}$$

Where τ represent the shear stress, THB corresponds to the yield stress determined using the Herschel-Bulkey Model. The other parameters are c as the Herschel-Bulkley viscosity, γ as the shear rate and p as the Herschel-Bulkley index (Anton Paar, n.d.).

The rheological properties of chocolate are found to be significantly affected by the particle size distribution, fat and lecithin present. Adding fat or lecithin or changing the particle size distribution can be used to control the chocolate quality (Afoakwa, Paterson, & Fowler, 2007, 2008; Afoakwa, 2016). Adding cacao-butter, which consists of fat and lecithin, steers the chocolate mass to a suitable viscosity (Beckett, 2008; González et al., 2021), whereas increasing the particle size distribution of the ground chocolate increases the yield stress.

Besides the flavour components of chocolate, the properties fat content and moisture contribute to the experience of the consumer. These properties influence the mouth-feel, melting behaviour and flavour release of the chocolate and are thus of great importance for the final chocolate quality (Stohner et al., 2012). The concentrations of fat content and moisture is usually determine using costly laboratory tools, which can delay the production process (Stohner et al., 2012). At Mars, Near Infra-Red Spectroscopy is applied on the chocolate sample to determine the fat and water concentration. NIR induces vibrational adsorptions in molecules by using the electromagnetic radiation in the near infra red spectral-range. Due the near infrared radiation, the sample molecules in the chocolate sample absorb photons and undergo a transition from vibrational state of lower energy to a state with higher energy (Stohner et al., 2012). Part of the light will be absorbed if a chocolate sample becomes irradiated with light of intensity I₀ and the emergent radiation I will be weaker. The absorbance (A=In (I₀/I)) can be defined by the LambertBeer Law in Equation 3 and is linearly related to the concentration c of the substance in the sample, where epsilon equals the molar extinction coefficient, l is the path length (Stohner et al., 2012). Using both equations, the concentrations for fat content and moisture can be determined.

$$A = ϵ * l * c$$

### 4.4 Current Control Practice

Literature mentions temperature time as an import parameter of the production process. Tempering consists of multiple heat exchanges and obtaining a set of standard tempering conditions is difficult due to the variable particle sizes and fat content. However, Afoakwa (2016) state certain tempering methods can still be used as to control the chocolate quality. It can reduce processing times while assuring a certain chocolate quality. Tempering and the conching phase times can thus be used to affect the chocolate properties, but both consume lots of power and thus also affect chocolate production costs (Tscheuschner & Wunsche, 1979; Sokmen & Gunes, 2006; Gonçalves & da Silva Lannes, 2010; Konar, 2013).

Consistent with literature in monitoring and controlling mixing processes, for Mars controlling the chocolate mixing process is a difficult task. The physical properties of chocolate include non-linear characteristics, which make it hard to grasp. Operators intervene the process based on their experience and each adaptation affects all four chocolate properties. Moreover, controlling the chocolate production process is either performed using at-line sensors at the end of the production cycle or relies on the experience of the operator.

### 4.5 Uncertainties Regarding the Production Process

In Veghel, operational decisions have been made which might impact the final physical properties of a chocolate batch. However, these impacts have never been explored and remain as a gut feeling.

### 4.6 Information Systems

A vast portion of this research relies on the knowledge which resides in the company. Stahmann and Rieger (2021) stress the importance of storing domain knowledge. Domain knowledge is not used for data generation, but can be used for the enrichment of data analysis. Domain knowledge is gathered throughout (short) semi-structured interviews with chocolate production operators or quality technicians. The domain knowledge and obtained knowledge from previous chapters, is used to identify all other relevant data sources. The daily manufacturing process of Mars is supported by several information systems (FactoryTalk VantagePoint, Sycon Subgroup Reports and SAP). Besides the daily operational function of these systems, the data stored here may also serve as an additional value for the process control. Unfortunately there is no such easily accessible system and all systems work on their own. As a result, it should be explored whether these information systems can be linked. In the next section, a brief description of the available data sources is given.

### Factory Talk VantagePoint

Stahmann and Rieger (2021) identified sensor data as the most relevant data source for data generation. Sensor data is recorded over time and can be used to generate time series. FactoryTalk VantagePoint is a business intelligence solution which integrates manufacturing (sensor) data stored in a historian database. This information system consists of machine log data of the whole factory through registering an enormous amount of PLC data. As a result, this information system provides wide access to unlabelled data of many different processing steps. For each conche machine, the system logs changes in batch codes, conche substatus (conche phase), storage tank and recipe. As this system only logs changes, there is no standard in between time. In addition, the amount raw materials present in the conche at each timestamp is estimated through a calculation. For each conche and for each batch, the usage of raw materials is registered. Also, the temperature of the chocolate mass and the total energy exerted on the chocolate mass is also registered in this system. For the main engine of the conche machine its revolutions, current and temperature is registered.

### Sycon Subgroup Reports

Sycon Subgroup Reports is a tool which registers the measured chocolate properties of a batch. As of March 2021, Mars changed its method to register the chocolate properties of batches. Before, only the viscosity, yield stress, fat content and moisture measurements and the actual timestamp of the measurement for all conches in a milling group were registered. The measurement did not include a batch identifier or was not directly linked to a specific conche. In case a batch was not first time right and required rework, multiple chocolate property measurements per batch are performed. Tracing these measurements back to the actual process data in VantagePoint was only possible using the timestamps. The possibility of multiple measurements per batch and all stored per milling group made batch traceability extremely sensitive to errors. As of March 2021, Sycon Subgroup Reports has improved and registers an AP_UBC batch identifier for each chocolate property measurement. Traceability of batches has improved through the introduction of the AP_UBC batchcode. Therefore, for this research only limited historical labeled data is available. The raw material usage and phase duration in Sycon Subgroups Reports is computed using the logged data retrieved from Factorytalk VantagePoint. As a result, this information can be seen as a summarization of the Factorytalk Vantagepoint and not as new or unknown source. Therefore, during the anomaly detection using forecasting methods, this information is not utilized.

### 5 Data Exploration

Factorytalk Vantagepoint stored many unlabelled data, whereas Sycon Subgroup Reports registers final outcomes of a batch. Combining the unlabelled data from Factorytalk Vantagepoint with the labeled data from Sycon Subgroup Reports will be considered as the main data source for this study. Therefore, the available data set consists only of process log data labeled with its final property measurement. Historical labeled data is only available as of March 2021 and is thus limited. In order to obtain largest possible sample size, data gathering was an ongoing process during this study. Eventually the process and property data of chocolate batches were gathered from the 19th of March until the 1st of October 2021. Outliers in terms of extreme batch duration, chocolate powder usage, faulty chocolate property measurements were removed. After removing outliers, a total of 1917 correctly labeled chocolate batches were explored during this study. The remainder of this chapter first explores the first measured chocolate properties and its relation to certain process characteristics. Afterwards, the distribution of faults and the characteristics over time are explored.

### 5.1 Chocolate Properties

For each conche, viscosity, yield, fat content and moisture are variable. Conches may produce chocolate batches with their median viscosity value above the target but still within the control limits. In general, it can be stated based on these four chocolate properties we observe little differences between the conches.

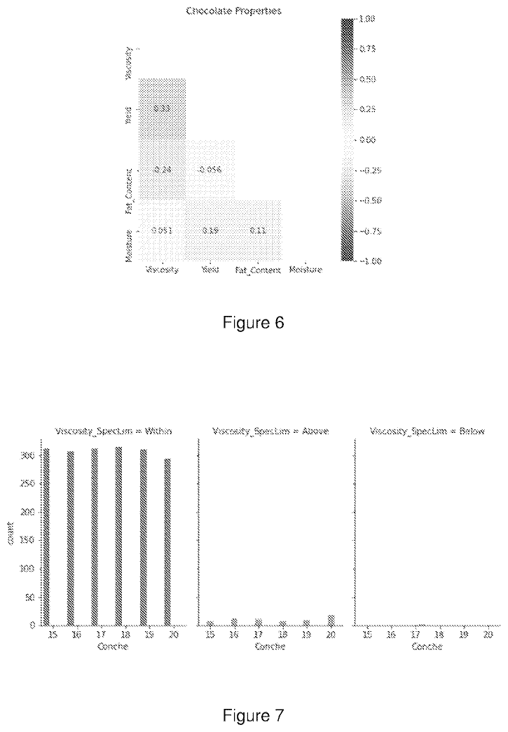

As mentioned in the previous chapter, the production of chocolate is complex. For data exploration the linear relationship among those four properties is explored. FIG. 6 provides an overview on how the four chocolate properties are related to each other. Within Mars the rheology is conceptualized by viscosity and yield, therefore we expect relationship between these two. It can be found that viscosity has a weak positive relation to yield (0.33). Further, viscosity is weakly negatively related to the fat content (0.24), which can be explained by the fact that fat is used to control the rheological properties. Moisture is weakly positive related to yield. Although the previous chapter described the chocolate properties as known to be non-linear, still weak relationships among those properties are found. This indicates that the chocolate properties are interrelated.

The first chocolate measurements seem to have little correlation and are little related. However, from the business understanding chapter it is known that adding certain raw materials or extending the duration of certain production phases affects the final chocolate property.

### 5.2 Distribution of Faults

Mars classifies their batches as right first time or as a fault based on the specification limits. The determination of the specification limits has been done using purely on domain knowledge. The production process is extended in case one of the four measured properties lies outside the specification limits. No process adaptations or rework is performed in case the chocolate properties are out of control. As a result, the specification limits are considered as more important. This section explores the faulty batches based on the specification limits.

FIG. 7 provides an overview of how the classification of the first measure of chocolate viscosity is distributed across different conche machines. Compared to all other measured properties, viscosity is the chocolate property with most out of specification limit observations. Therefore, the bar chart easily provides a first indication of the sparseness of faulty occurrences. As can be seen in FIG. 7, a total of 65 faulty batches are related due to a too high viscosity, while only 5 batches of chocolate are related to a too low viscosity. Quality technicians expect possible differences between uneven and even numbered conches to the dosage insecurity. However, this Figure does not confirm this expectation for viscosity as the faulty viscosity faults are about evenly distributed over all different conches, with a slightly higher value for conche 20.

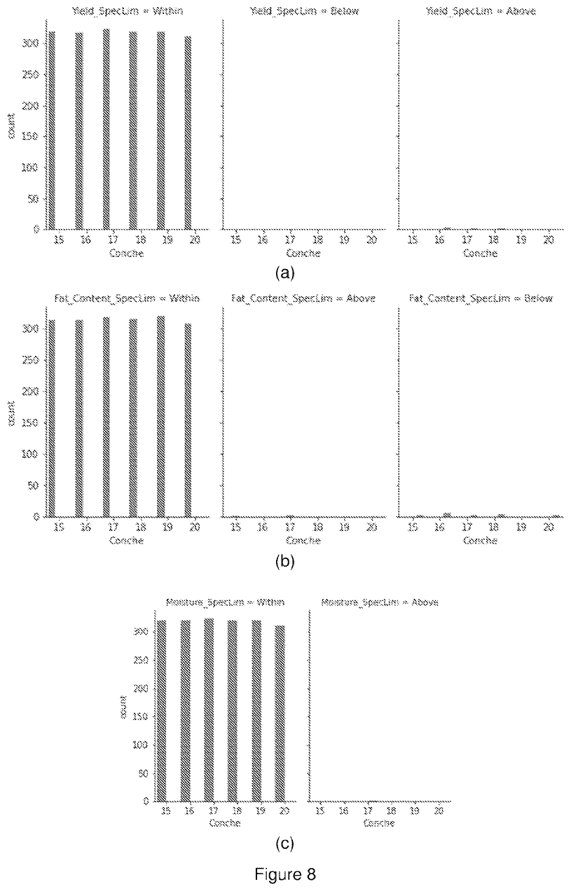

Similarly, FIG. 8 shows bar charts for respectively yield, fat content and moisture. It shows how the occurrences of above, within or below specification limit chocolate batches are distributed across the different conche machines. Results of yield are shown in FIG. 8a. The Figure shows only nine faulty batches occurred due to yield of which seven consist of a too high yield and only two batches have a too low yield. Chocolate batches with a yield below specification limit only occurred at conches 15 and 19, whereas a too high yield occurred at conches 16, 17 and 18. Surprisingly, the below specification and above specification limits occurred at different conches. Supporting the gut feeling of existing differences between conches due to the supply of chocolate powder. However, due to the very little occurrences this gut feeling cannot be tested and remains a gut feeling. Compared to the yield value, more faulty batches are observed due to a faulty fat content value. As shown in FIG. 8b, on all conches in total of 23 chocolate batches with yield value below specification. Chocolate batches with too low fat content are evenly distributed over conches 15, 16, 17, 18 and 20. In addition on conche 15 and 17 also seven chocolate batches with a yield value above specification limit were produced. FIG. 8c illustrates batches with a faulty moisture value, occurred very rarely as only three batches with a too high moisture value occurred only on conche 17.

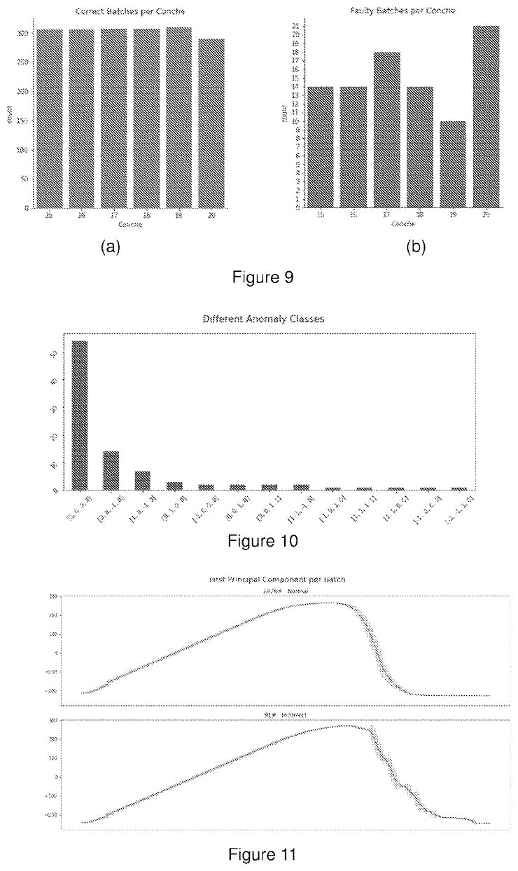

Due to the small number of observations no statement about the gut feeling regarding differences between even and uneven conches can be made. The little occurrences of faulty chocolate batches illustrate the sparseness of the problem. FIG. 9 shows the how the faulty batches are distributed across the six different machines if the faults are grouped and batches are binary labeled. Then in total of 1826 correct batches and only 91 faulty batches are found. All conches produced about 320 batches of chocolate. Conche 20 produced around 290 and also has the highest number of faulty batches, as a result this conche has the highest failure rate. Further, FIG. 10 demonstrates how the different fault classes are distributed. Each property can take up 3 values, either below, within or above specification limits and in total of 3*3*3*3=81 possibilities exist. However, as shown in FIG. 10, only 13 different types of anomalies and one normal sequences are found due to the limited sample size. The figure additionally shows more than 50 percent of all anomaly occurrences belong to one specific anomaly type. These chocolate batches have an viscosity value above specification limits.

The sparseness does introduce another challenge. Given a sufficient amount of anomalous samples, classification seemed to be the straightforward approach for pattern recognition in time series data. Then sequences get predicted as normal or as a specific fault. Supervised learning heavily relies on high quality of data, implying sufficient and qualitative labels. Therefore, standard machine learning algorithms often perform bad on imbalanced data sets. These algorithms rely on the class distribution to make predictions and learn that the minority class is not as important as the majority class. Due to the sparseness of fault chocolate batches together with the interrelated properties it is chosen to frame the project as an anomaly detection problem. Anomaly detection can be seen as form of pattern recognition.

5.3 Different characteristics between Normal and Faulty Batches

It is explored whether the normal (between specification limits) batches consist of distinctive characteristics from batches with chocolate properties out of specification limits. In standard situations, filling a conche with the raw materials should take approximately slightly less than one hour. However, during the data exploration phase samples with an extreme high filling duration were found. In few cases, when demand for chocolate is low, the choice is made to keep a conche machine as unused. However, it could happen that the machine had started filling the conche already with very little chocolate powder, after which it was turned off. For these cases, vantage point registers the machine incorrectly as started. As a result the filling duration could take up as long as it is used again. The choice has been made to remove such extreme samples. In order to keep the sparse set of anomalous samples, the choice has been made to include samples with a filling duration up to 2 hours. A filling duration above 60 minutes indicates either the machine has been paused during filling or the output of grinding machine was lower and filling took longer.

After filling the conche with raw materials, the dry conching phase starts. The dry conching duration is considered as the primary phase where chocolate characteristics are developed. For normal samples, the average time until this phase is finished centers around a particular time after commencement, whereas for the anomalous samples this value is as expected a bit higher. The conche machine automatically adapts duration of certain cycles. Therefore, and as expected, the production cycle of faulty batches is observed to be longer compared to the RFT batches.

### 5.4 Time Series Exploratory Analysis

Monitoring and controlling the chocolate production is known to be challenging due to its non-linear characteristics. Still, the FIG. 6 showed weak and moderate relationships. However, these exploration methods just utilize certain point characteristics and do not consider the time-dependence within the features. Therefore, this section checks for different patterns between Right First Time and faulty chocolate batches. For each numeric feature over time, the smoothed out average, with its standard deviation above and below is plotted over time. During the informal interviews with the chocolate production operators and quality technicians, it was mentioned conche process is fully automated. As a result, it is expected that anomalous batches show distinctive patterns from normal batches.

Normal and anomalous batches both show similar variance during the first phase. After this phase, in normal cases the dry conching phase should start. As earlier mentioned, within Mars dry conching is known as the main chocolate production phase, during which the chocolate properties develop. As a result, it was expected this phase shows more variance. However, no distinctive patterns between normal and anomalous batches are found until the end of the production cycles. This variance at the end of the production cycle might be induced by the extension of the dry conching phase and is thus not informative.

It can be concluded, looking at smoothed out averages of the sensor features, little differences between normal and anomalous chocolate batches are found. Literature already describes the chocolate production as a complex process and purely using single features is not sufficient to describe its quality. If differences in patterns are found at all, then these mainly occur at the end of the production cycle. However, for Mars predicting the quality of a batch at the end of the cycle is not interesting as it is standard practice to measure chocolate properties here.

5.5 First Principal Component over Time

Alternatively, by calculating the first principal component using Principal Component Analysis (PCA) the multivariate sensor channels may be reduced to univariate time series (Malhotra et al., 2016). Using the first principal component, a certain amount of variance from the original sensor channels is captured. As a result only one scalar value has to be considered per time-step, which can simplify the complexity of a neural network for anomaly detection (Malhotra et al., 2016). However, in this study, reducing the sensor channels will only be utilized as an exploratory manner. Detecting unexpected behaviour in the reduced dimension does not allow retracing the origin of the anomaly in the original channels.

Similar to the single features as described in previous section, it is checked whether the smoothed out average of the first principal component is distinctive between normal and anomalous batches. The smoothed averaged results of the first principal component analysis are shown in FIG. 11. For this comparison all sequences have been zero-padded to a fixed sequence length. The first principal component shows similar characteristics over time, but vary slightly in position and duration, such that each sequence is unique. Compared to the smoothed patterns for most single features, where no variance was visible after filling the machine, the first principal component does induce slightly more variance during dry conching for both normal and anomalous sequences. Variance is visible as the light blue surface surrounding a line. Unfortunately, the smoothed average first principal component shows no distinctive patterns until the end. At the end a slightly distinctive pattern is found. However, this distinctive pattern might be smoothed out whenever the sample size of anomalous patterns increases. The law of large numbers states that an observed sample average from a large sample will be close to the true population average and that it will get closer the larger the sample. As a result, anomaly detection using solely the first principal component of the sensor channels, as described by Malhotra et al. (2016), is not sufficient for detection of anomalous patterns.

### 5.6 Concluding Remarks

During the data exploration several useful insights were gained. Although the chocolate properties includes non-linear characteristics and require expensive laboratory tools to measure them, exploratory analysis showed the four chocolate properties are weakly related to few process characteristics. The weak relationships demonstrate the inputs are related to the output and provide an indication these features can be used for predicting the quality of such a complex substance. Moreover, the exploratory data analysis showed the data is highly imbalanced and some faults in terms of chocolate properties happen more frequently than others. As an example the most occurring fault is exclusively related to a too high viscosity value and represents 60 percent of all faults. The availability of anomalous sequences limits the modeling possibilities. As a result it was chosen to combine the different fault classes into one and explore these two categories. Data exploration regarding difference in patterns of RFT and faulty sequences revealed the difficulty of the faced problem. These two groups showed very little differences in the smoothed average of a single feature or the first principal component.

### 6 Quality Modeling

This chapter describes the development of a predictive model by addressing the data preparation and modeling phase of the CRISP-DM framework. The data understanding and business understanding were used to select data features that serve as input and output to the forecasting model. The data selection approach is listed in Section 6.1. The choice of final model architecture affects some data preparation decisions such as scaling and encoding. Therefore during data preparation, which is explained in Section 6.2, the type of models that are to be developed are already considered. The aim of the model is early detection of anomalous production cycle patterns, and thus concerns time-series data. As such, a dataset with time-series sequences will be generated in Section 6.3. Section 6.4 explains how different sequence-to-sequence are developed and how these can be applied to detect anomalies. Finally, Section 6.5 explains how the sequence-to-sequence models can also be applied in deep hybrid models to detect anomalies. The deep hybrid models utilize the sequence-to-sequence output as input to supervised classification algorithms.

### 6.1 Data Selection

The first step in the data preparation phase of the CRISP-DM framework is the data selection step. Data selection is concerned with selecting the data features that will be used in the machine learning model. Results of the data selection step is a set of data features that are relevant to the machine learning model. A total of 21 features is used, which are summarized in Table 4.

### 6.1.1 Data Features

As found in Chapter 5, different related data sources can be considered when determining data features. Timeseries data regarding the current production process can be retrieved from Vantagepoint, whereas the final production results are registered in Subgroups reports. Because Vantagepoint include a vast amount of data, a careful selection of which data to use is required. This requires domain experts' participation and provides an opportunity to incorporate their knowledge into the data (Guyon and De, 2003). The operators and quality technicians of Mars Veghel are domain experts and through several interviews with them a set of available data features has been constructed. These data features are expected to be associated with chocolate quality based on the interviews and chocolate manufacturing literature in Chapter 4. Table 4 provides an overview of the selected data features.

TABLE 4
Timeseries Data Features

| Type | Name | Explanation |
|---|---|---|
| General | Time | Actual Time |
| | Conche | Conche identifier |
| | Batchcode | Chocolate batch identifier |
| Production | Substatus | Current production phase category |
| Phase | | |
| Raw | Total_1 | Used quantity until this time |
| Materials | | |
| | Total_2 | Used quantity until this time |
| | Total_3 | Used quantity until this times |
| | Total_4 | Used quantity until this time |
| | Total_5 | Used quantity until this time |
| Main Engine | Current_MainEngine | Electrical current of the main engine in |
| | | Ampere |
| | Temperature_Frequency_Control | Temperature of the Frequency Controller |
| | | in Celsius |
| | Power_MainEngine | Electrical power of the main engine in kilo |
| | | Watt |
| | Temperature_Mass | Temperature of the mass in Celsius |
| | Revolutions_MainEngine | Revolutions per Minute |
| Controller | MainEngine_Control | Binary indicating whether the main engine |
| | | is active |
| | MainEngine_Control_Direction | Binary indicating whether direction |
| | | changes |
| | 1_Door_Control | Binary indicating whether 1 is added |
| | 2_Door_Control | Binary indicating whether 2 is added |
| | Boiler_Casing_Heat_Control | Binary indicating whether heating water is |
| | | used |
| | Boiler_Casing_Cool_Control | Binary indicating whether cooling water is |
| | | used |
| | Chocolate_Supply_Control | Binary indicating whether old chocolate is |
| | | added |
| | 3_Door_Control | Binary indicating whether 3 is added |
| | 4_Door_Control | Binary indicating whether 4 is added |
| | 5_Door_Control | Binary indicating whether 5 is added |

### 6.1.2 Target Variable

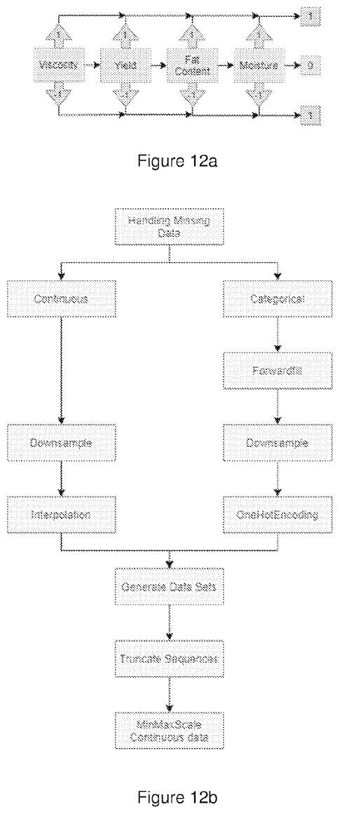

Within Mars a chocolate batch is registered as Right First Time or as incorrect. Every sequence is measured on four different properties; viscosity, yield, fat content and moisture. Based on these four properties a chocolate batch can either be in control, out control but within specification limits or out of specification limits. Right first time chocolate batches include all batches within specification limits and require no additional work. Most importance is assigned to out of specification limit batches, because these chocolate batches require additional work. Therefore, the data preparation started with first determining whether for each property the batch of chocolate was either below, within or above specification limits. Similar approach was used to determine whether the chocolate batch is in control. Each sequence is scored on all four chocolate properties by assigning a −1, 0, or 1. The value 0 indicates the chocolate batch was within specification, and (minus) 1 indicates it was either above (below) limits. During the data exploration it was found the number of sequences with a chocolate property outside the specification limits was quite sparse. Therefor, as shown in FIG. 12a, it is chosen to label each sequence with a binary value which indicates an incorrect chocolate batch. The binary value combines all four different chocolate specification limits, as shown in FIG. 12. In total of 3*3*3*3=81 possibilities exist, however due to the limited sample size only 13 different types of anomalies and one normal sequences are found. Eventually, and because the project is framed as an anomaly detection problem, the final outcome variable is either a 0 for normal sequences or a 1 for anomalous sequences.

### 6.2 Data Preprocessing

The pattern during which the anomaly occurs is unknown and might span over many minutes within the cycle. As a result, an enormous amount of data points per production cycle should be handled. Recent publications described in Section 2.3 utilized time series windows of sensor sequences with less than 500 data points for pattern recognition.

Additionally, the sampling rates of sensors and controllers are different and even vary between different conches. Therefore, re-sampling at a lower but fixed rate compared to the original data sequences is a crucial part of pre-processing for detection of anomalies in parts of the production cycle. An overview of the applied pre-processing steps is illustrated in FIG. 12b and the steps are further explained below.

Due to the different sampling rates many missing values are found. There are several possibilities to overcome this issue. First, continuous and categorical data types should be handled differently. The categorical features comprise the Conche, Substatus and all controller features. Conche and Substatus of the machine are both categorical values and the controller features are a binary indicating whether a certain function is active. For both types of features it is assumed the value remains the same until the next change. Therefore, missing categorical values are handled by forward filling the categorical values. Afterwards the time series is down-sampled to one sample per minute by taking the last value of each minute and finally OneHotEncoding is applied for the conche and substatus categorical features.

For numeric values a different pre-processing strategy is used because forward filling would lead to incorrect results. In general, with neural networks, it is assumed to be safe to input missing values with 0, as long that the 0 value does not have a meaningful value (Chollet, 2018). In this case it cannot be guaranteed that the zero does not have a meaning value. For example, in case of the raw material usage features, a value of 0 implies no raw materials are used while this is actually not true. Therefore, for the numeric values the time series is first down sampled by taking the average value to one sample per minute. Afterwards, missing values of the minutes whenever the machine was active are imputed by linearly interpolation. Once missing values are handled, the minutes whenever the machine was inactive is discarded. These minutes are discarded because the target value considers the chocolate properties of a batch of chocolate and inactive minutes are not labeled. Then the categorical and continuous data are merged. In order to generate the final time series sequences for predictive modeling, all minutes are grouped on its unique batch code. Resulting in multiple sequences where each sequence has shape (Minutes, Features) and is labeled with its chocolate properties.

LSTM autoencoders will be utilized as anomaly detection models, which will be explained in Section 6.4. A major benefit of such a combined model is that it eliminates the need for preparing hand-crafted features and facilitates the possibility to use raw data with minimal pre-processing for anomaly detection tasks (Chalapathy & Chawla, 2019).

### 6.3 Data Generation

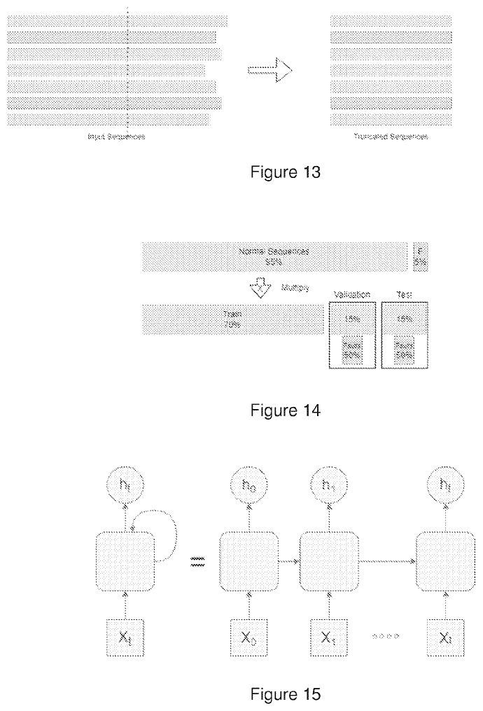

The multivariate input sequence from the given dataset have varying lengths, because the conche machine automatically adapts to the current cycle. As mentioned earlier, the decrease current of the main engine during the dry conching phase determines whether the conching cycle is extended or not. In addition, certain qualities of raw materials can cause the production cycle to have different characteristics, for example different quality of cacao butter can either smooth the particles or generate more resistance, and therefore result in an alternating sequence lengths. In literature, often sequences are padded with zeroes on the end to generate sequence with equally lengths. Literature then uses the full sequences to detect anomalies. However, for the case at Mars, it is not interesting to use the full sequence for prediction as it is standard practice to measure the four qualitative properties at the end of the cycle. Another possibility is using a sliding window approach, where for each sequence multiple sliding windows are generated. However, this induces another challenge because labels are only known for the whole sequence, and not for a part within the sequence. Therefore, it is chosen to truncate sequences after a certain amount of time. This process is illustrated in FIG. 13. Truncating sequences at a particular time enables an early anomaly detection. In addition, truncating ensures no sequences should be padded with zeros. The effect of having longer sequences is also explored by extending the sequences with either one or two hours.

Anomaly detection is often performed using autoencoders. An autoencoder learns to reconstruct normal sequences. Afterwards, anomalies can be detected through calculating an anomaly score based on the differences between the original and the reconstructed sequence. The function to calculate the anomaly score will be specified in another section. Different splits of data should be generated in order to learn the right behaviour. Therefore, the data will be randomly split as described in FIG. 14. It is chosen to use random splits because the chocolate production samples are individual occurrences. Splitting the data into a training, validation and test set ensures the model's predictive performance is tested on an unseen test data set. Training of the AutoEncoder model is done in a semi-supervised manner, where first each model is trained unsupervised exclusively on normal data. Once, training the autoencoder is done, the validation set is used for supervised parameter tuning by setting an error threshold. Hence, generating the data splits starts with generating a normal dataset and an anomalous data set. FIG. 14 illustrates the training set consists exclusively of 70% of all normal samples. The remaining normal samples and anomalous samples are evenly split into a validation and test set. Additionally, the chocolate production cycles are independent. Therefore, it is chosen to randomly split the normal and anomalous sets. An overview of the resulting sample set sizes is given in Table 5.

TABLE 5

| Sample set sizes | | | | |
|---|---|---|---|---|
| Type | Train | Validation | Test | Total |
| Normal | 1278 | 274 | 274 | 1826 |
| Anomaly | — | 45 | 46 | 91 |

### 6.3.1 Scaling the Data Sets

Before training a Neural network, the data may be scaled. Without scaling, if a feature is big in scale compared to others, then this big, scaled feature might become dominating and, as a result of that, predictions of the Neural Network may not be accurate. Further, models converge slowly without scaling because calculating of output might require a lot of computation time and memory. In order to prevent data leakage, scaling the data must be performed after splitting the data. Implying that only training sequences are used to fit a scaler. Afterwards, the same scaler is applied to the validation and test set. Literature suggests not to scale the measurements of sensors using standard normalization as these do not typically follow the normal distribution and scaling them might result into a loss of information (Sapkota, Mehdy, Reese, & Mehrpouyan, 2020). In addition, literature suggests to treat the measurements of actuators differently from sensor measurements. As a result it is chosen to only scale sensor measurements using Min Max scaling. Min-max normalization retains the original distribution of scores except for a scaling factor and transforms all the scores into a range between 0 and 1. One disadvantage of min max scaling, is that it is highly sensitive to outliers. Therefore, before spitting the sequences into the train, validation and test set the sequences with extreme outliers in terms of batch duration, chocolate powder usage, faulty chocolate property measurements were removed.

### 6.4 Sequence-to-Sequence Modeling

This section explains the sequence-to-sequence modeling techniques which are applied during this study. First the working of recurrent neural network units, in Section 6.4.1, and long short-term memory units, in Section 6.4.2, are explained. Thereafter, Sections 6.4.3, 6.4.4 and 6.4.5 demonstrate how these units are utilized to construct sequence-to-sequence models and how these can be utilized to detect anomalies. Finally, Section 6.4.6 describes how the architecture and parameters of the autoencoders can be optimized.

### 6.4.1 Recurrent Neural Networks Units

Recurrent Neural Network (RNN) is a subclass of Artificial Neural Networks designed to capture information from sequences or time series data. In a normal feed forward neural network signals flow in only one direction from the input to the output at a time. Contrary, a recurrent neural network is capable of receiving a sequence as input and can produce a sequence of values as output. Recurrent Neural Networks are capable of capturing features of time sequence data (Williams 1989).

Recurrent neural networks take as input not just the current input data, but also considers what has been perceived previously in time. An RNN maintains a hidden state vector which acts as a memory and preserves information about the sequences. Long-term dependencies between events are memorized through the hidden state. This allows the recurrent neural network to use simultaneously the current and past information for making a predictions. The structure of an RNN is illustrated in FIG. 15. Recurrent neural networks can effectively incorporate temporal dependencies within input data. It captures these dependencies by unrolling the temporal axis. As shown in FIG. 15, at each time step the network is provided with feedback connections from the previous time steps. The process works as follow: The output of a recurrent neuron, at given time step t, is a function of the previous input, which can be considered as a mechanism of memory. Each neuron has an output ŷₜ and a hidden state hₜ. The hidden state of a recurrent neuron is passed to the next time step neuron as shown in Equation 4

$${h}_{t} = g ( {h}_{t - 1} , {x}_{t} )$$

In order to train RNNs an adaptation of the normal Backpropagation called Backpropagation Through Time (BPTT) is used. BPTT works as follows; First, all time steps are unrolled, then each time step has one input time-step, one copy of the network, and one output. The loss is calculated for each time step and is accumulated. Once all timesteps are processed, the network is rolled back up and weights are updated accordingly. However, Hochreiter (1991) discovered classical RNNs suffer from the vanishing gradient problem, which is caused by the feedback loops inside the hidden layers. The vanishing gradient problem limits capabilities of RNNs to learn dependencies over long intervals (Chalapathy & Chawla, 2019). In order to overcome the vanishing gradient problem, Hochreiter and Schmidhuber (1997) developed a Long Short-Term Memory (LSTM) network.

### 6.4.2 Long Term Short-Term Memory Unit

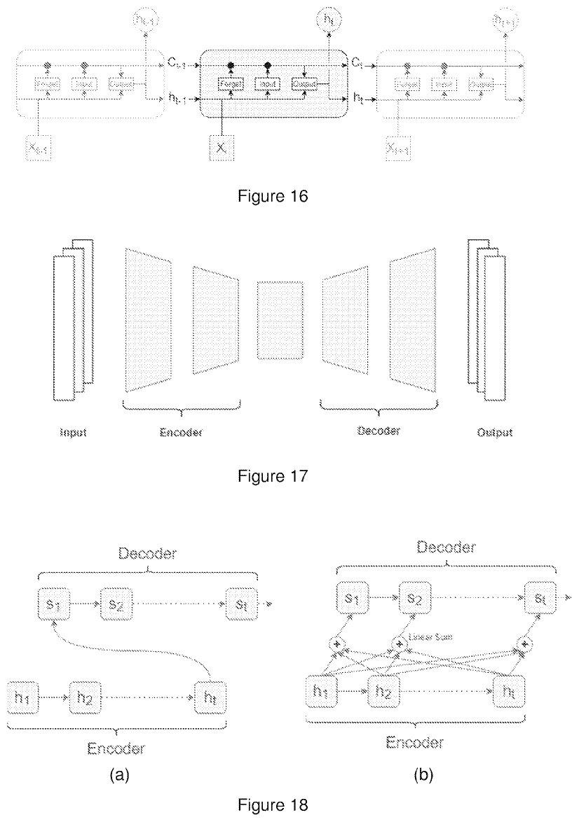

Hochreiter and Schmidhuber (1997) introduced an adaptation of classic RNNs to overcome its issues. Their introduced network is called the Long Short-Term Memory network (LSTM). Since its introduction, the networks have evolved and are now the most popular types of RNNs. LSTM is better capable of learning long term dependencies over substantial long time intervals without being affected by the vanishing or exploding gradient problem. The architecture of RNNs, as illustrated in FIG. 15, is adapted. Gates are activation functions, which can add or remove information from the cell state. The classical RNN acts as itself as a sole gate for the data manipulations. The classic RNN neuron and its feedback loop is replaced with an LSTM unit. An LSTM unit has a more complex structure composed of four gates. An LSTM unit is a gated cell which contains information outside the flow of an RNN. The memory of an LSTM is the cell state. The LSTM unit decides what to store on the cell state. Using gates, which can be opened or closed, it determines when the cell state can be read, written or deleted. Gates are opened or closed based on a signal. The signals strength determines whether information is passed or blocked. Similar to classic RNNs, BPTT learning is used to adjust and optimize the weights associated with the gates, such that the LSTM network learns when to allow the reading, writing or deletion of information. A simplified representation of an LSTM unit is illustrated in FIG. 16.

The first layer in the unit is called the forget layer, which takes as input the new information of the current time step Xₜ and the output of the previous time step (hₜ−1). Using this input, the forget layer decides which information to forget from the cell state of the previous time step (Cₜ−1) through the forget gate and computes its own cell state (Cₜ). The input layer then decides what new information will be stored on its own cell state. The input layer decides which values to update and with how much the values have to be updated through the input gate. Finally, in the output layer, using the output gate, the unit decides on the output (hₜ). The output is a filtered version of the updated cell state and its current input. Summarizing, the forget gate controls the extent to which a value remains in the cell state, the input gate controls the extent to which a new value flows into the cell state and the output gate controls the extent to which the value in the cell state is used to compute the output of the LSTM unit.

LSTMs have been proven to perform well in many recent publications and are rather easy to train. Therefore, LSTMs have become the baseline architecture for tasks, where sequential data with temporal information has to be processed. As an example, Chalapathy and Chawla (2019) state RNN and LSTM based methods show good performance in detecting interpretable anomalies within multivariate time series datasets.

### 6.4.3 Autoencoders

In this section three different autoencoders are introduced; a normal autoencoder, an autoencoder with an attention mechanism and a variational autoencoder. An autoencoder is composed of an encoder network and a decoder network and its structure is illustrated in FIG. 17. The encoder maps the original data onto a low-dimensional feature space, whereas the decoding network attempts to reconstructs the data from the projected low-dimensional space. A reconstruction loss function is used to learn the parameters of these two networks. A bottleneck architecture is often used to enforce the autoencoder to learn important information for reconstructing the data (Pang & Van Den Hengel, 2020). Different types of autoencoders are available and the architecture choice depends on the nature of the data. Convolutional Neural Networks are preferred for image datasets, whereas the LSTM based models show to have good performance for time series data (Chalapathy & Chawla, 2019; Pang & Van Den Hengel, 2020). As a result, during this study LSTM autoencoders are used. Of course, other autoencoders may be used in their place. A major benefit of using an LSTM-autoencoder is that it eliminates the need for preparing hand-crafted features and thus facilitates the possibility to use raw data with minimal pre-processing for anomaly detection tasks (Chalapathy & Chawla, 2019).

### 6.4.3.1 Binary Autoencoder Classification Using a Threshold

Although autoencoders are trained in an unsupervised manner, these methods can still be used as a binary classifier. After learning the normal behaviour by training the autoencoder on exclusive normal behaviour, the validation set enables to distinguish normal samples from anomalous samples. The autoencoder reconstructs each sample and the reconstruction can be used to calculate the mean reconstruction error. It is assumed that the reconstruction error of normal labeled samples differs from anomalous samples, where for normal samples the error should be low and high for anomalous samples (Pang & Van Den Hengel, 2020). Different possibilities for the reconstruction error exist such as the Mean Absolute Error (MAE) or Mean Squared Error. In order to classify new data samples, based on the validation set a threshold t must be set. The threshold is then used as cutoff point and the test set is used to evaluate the performance of the reconstructing autoencoder and its chosen t. When the errors are normally distributed, t can be determined by utilizing the standardized Z-scores. Z-scores enable the use of percentiles to set a threshold and points are considered as outliers based on how much they deviate from the mean value. However, the mean is also affected by outliers. Instead of using the mean, the Median Absolute Deviation (MAD) is less affected by outliers and thus more robust (Rousseeuw & Hubert, 2011). MAD is defined as the median of the absolute deviations from the data's median X, see Equation 6. The modified Z-score is then calculated with the MAD instead of the standard deviation, see Equation 7.

$$\tilde{X} = median ( X )$$

$$M A D = median ( \vert {X}_{i} - \tilde{X} \vert )$$

$${M}_{I} = \frac{0.6745 ( Xi - {X}^{\sim} )}{M A D}$$

These Z-scores can then be used to determine when a sample is an outlier or not, setting the threshold τ based on the standardized distribution. Alternatively, if errors are not normally distributed τ can be determined using the precision and recall curve of the validation set. Depending on the anomaly detection task, this method provides more flexibility in terms of favouring either recall or precision. Using thresholds gives the model some flexibility, but choosing the optimum threshold value is a difficult task which requires thorough validation to avoid over- or under-fitting.

Using this approach, enforces the autoencoder to learn important regularities of the normal data in order to minimize the reconstruction error. Pang and Van Den Hengel (2020) state advantages of using data reconstruction methods include the straight forward idea of autoencoders and its generic application to different types of data. However, the learned feature representations can be biased by infrequent regularities and the presence of outliers or anomalies in the training data. Besides, the objective function during training the autoencoder is focused for dimensionality reduction rather than anomaly detection. As a result the representations are a generic summarization of the underlying regularities, which are not optimized for anomaly detection (Pang & Van Den Hengel, 2020). Even though an LSTM unit performs better compared to a classic RNN network, classical LSTM autoencoders still suffer from long sequences. In a classical autoencoder, the entire sequence is encoded using the last hidden state at the last time step (Dai & Le, 2015; Kundu et al., 2020). In case the sequence is long, the encoder will tend to have much weaker memory about earlier time steps. Then the encoded state is often not sufficient for the decoder to produce a good reconstruction. An attention mechanism could solve this problem (Bahdanau, Cho, & Bengio, 2014), therefore the use of attention weights for anomaly detection is also considered.

6.4.4 Autoencoder with Attention Mechanism

Similar to Kundu et al. (2020), producing chocolate can be seen as an active process because the system automatically adds lecithin as a response to its trend. As a result it is logically to assume, the future is influenced by the past. An attention mechanism assigns weights to every input sequence element contribution to every output sequence element and enables to encode past measurements with its required importance to the present measurement (Kundu et al., 2020). Attention Mechanism for sequence modelling was introduced by Bahdanau et al. (2014). The authors used the attention mechanism to translate an English sentence to French and describe the main issue of classical autoencoders that it needs to be able to compress all the necessary information into a fixed-length vector. The fixed-length vector makes it difficult for the neural network to cope with long sequences. As explained in previous section, in a normal autoencoder architecture the decoder reconstructs the input by looking exclusively at the final output of the encoder step. Contrary, an attention mechanism helps to look at all hidden states from the encoder sequences. A reconstruction is generated after the mechanism has decided which hidden state is more informative. For both types a simple architecture is illustrated in FIG. 18. The Figure explains the difference between a normal and attention based autoencoder.

Basically, two different types of attention exist. First, additive attention was developed by Bahdanau et al. (2014). Based on the idea additive attention, Luong, Pham, and Manning (2015) further developed multiplicative attention. The two attention mechanisms differ when the attention mechanism is introduced in the decoder and in the way the alignment score is calculated. Additive attention uses the attention mechanism at the end of the decoding process, whereas multiplicative attention uses the RNN in the first step of the decoding process. Further for multiplicative attention three alignment score calculation methods, as explained below, exist. For simplicity during this research only one autoencoder architecture with multiplicative attention and dot alignment score is employed. Multiplicative attention starts with producing the encoder the encoder hidden states of each time step in the sequence. Iterating over each time step, the decoder utilizes the previous decoder hidden state and output to generate a new decoder hidden state for current time step (Luong et al., 2015). The full process can be best explained using the following steps:
1) For each time step t, the model starts with producing the encoder hidden state h̅ₛ
2) Then the model derives a variable-length alignment weight vector aₜ, whose length equals the number of time steps on the source side. The alignment vector is computed by scoring the current target hidden state hₜ with each source states h̅ₛ.

$${a}_{t ( s )} = \frac{exp ( score ( {h}_{t} , {\overline{h}}_{s} ) )}{{\sum }_{{s}^{′}} ( exp ( score ( {h}_{t} , {\overline{h}}_{s} ) ) )}$$

Three different alternatives of scoring are considered. These are given in Equation 9; the dot scoring function is considered as the simplest scoring function.

$$score ( {h}_{t} , {h}_{s} ) = \{ \begin{matrix}{h}_{t}^{T} {\overline{h}}_{s} & dot \\ {h}_{t}^{T} {W}_{a} {\overline{h}}_{s} & general \\ {W}_{a} [ {h}_{t}^{T} ; {\overline{h}}_{s} ] & concat\end{matrix}$$

3) After computing the alignment weights, the alignment weights are softmaxed to ensure that all weights are between 0 And 1.

aₜ=softmax(aₜ)
4) The dot product between the encoder hidden states (h̅ₛ) and attention weights (aₜ) is taken to compute the context vector cₜ.
5) Finally the context vector is concatenated with the decoder hidden states, which is then used as input to the last layer to make a reconstruction.

However, the normal and attention autoencoders, as described above, might not be able to grasp the non linear production process characteristics. Recently, Variational autoencoders have been developed as a deep generative model, which is known as a powerful method for learning representation from data in a non linear way. It exploits information in the data density to find an efficient lower dimensional feature space as a multivariate normal distribution (An & Cho, 2015; Suh et al., 2016). Therefore, it is explored whether variational autoencoders are better at detecting anomalous chocolate batches.

### 6.4.5 Variational Autoencoder

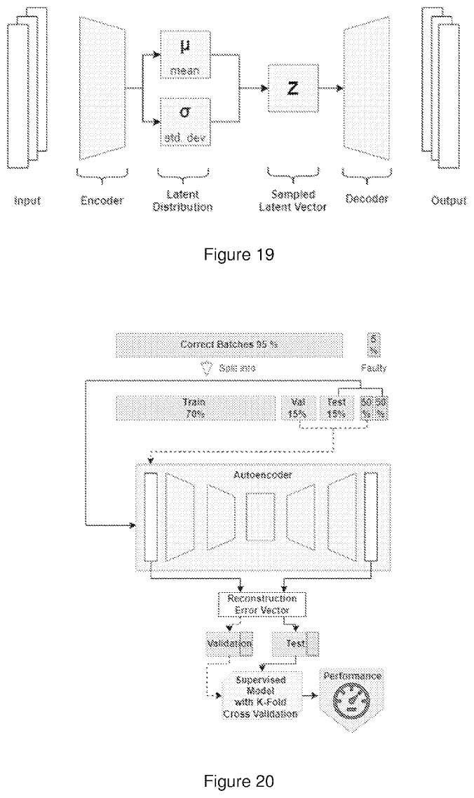

A variational autoencoder is a Bayesian neural network which does not try to reconstruct the original sequence, but tries to reconstruct the distribution's parameters of the output. A normal autoencoder encodes a smaller representation of the original input by learning a smaller representation. The decoder then reconstructs the original sequence from this smaller representation. Within VAE context, the smaller representation is known as a latent variable and has a prior distribution. For simplicity often the Normal distribution is chosen. A sequence is encoded into a mean and standard deviation of the latent variable. Then a sample is drawn from the latent variable's distribution. The decoder decodes the sample back into a mean value and standard deviation of the output variable. The sequence is reconstructed by sample from the output variable's distribution. The architecture is illustrated in FIG. 19. The full process can best be explained as below.

In Bayesian modelling, it assumed the distribution of observed variables are governed by the latent variables. Usually, only a single layer of latent variables with a Normal prior distribution is used. Let x be a local observed variable (sequence) and z its corresponding local latent variable. The probabilistic encoder, which is known as the approximate posterior q_{ϕ}(z|x), encodes observation x into a distribution over its hidden lower-dimensional representations. For each local observed variable xₙ, the true posterior distribution p(zₙ|xₙ) is approximated over its corresponding local latent variables zₙ. A common approach is to approximate it using a variational distribution qϕₙ(zₙ|xₙ), specified as a diagonal Gaussian, where the local variational parameters ϕₙ={μₙ, σₙ}, are the mean and standard deviation of this approximating distribution. Finally, the vector z is sampled by the encoder part of the VAE.

The decoder decodes the hidden lower-dimensional representation z into a distribution over the observation x. This joint distribution p_{θ}(x|z) is defined as a multivariate Bernoulli whose probabilities are computed from z using a fully connected neural network with a single hidden layer. The Negative Log Likelihood of a Bernoulli is equivalent to the binary cross-entropy loss and contributes as the data-fitting term to the final loss.

The variational autoencoder loss function is composed of the reconstruction loss, as explained above, combined with the KL divergence loss. The combination between reconstruction loss and the KullbackLeibler (KL) divergence ensures that our latent space is both continuous and complete. Further, gradient optimization requires that the loss function can be differentiated. However, this is not possible for variational autoencoders because the loss of VAEs depends on the parameters of the probability distribution. Therefore, Monte Carlo estimation using the reparameterization trick developed by Kingma and Welling (2013) is applied. Of all estimation methods, the reparameterization trick has been shown to have the lowest variance among competing estimators for continuous latent variables (Rezende, Mohamed, & Wierstra, 2014). The reparameterization trick samples the value for z using the computed μ And σ.

$$z = μ + {σ}^{2} * ϵ$$

### 6.4.6 Optimizing Autoencoder Hyperparameters

Hyperparameter tuning is considered as the key for machine learning algorithms. The goal of hyperparameter optimization is to find a set of parameters a predefined loss function on independent data (Claesen & De Moor, 2015). The optimal hyperparameters should avoid under-fitting, where both training and test error are high, and over-fitting where the training error is low, but test error is high. Carneiro, Salis, Almeida, and Braga (2021) state searching a grid with different sets of parameters is a method to find the best parameters of a neural network, as such grid-search is used.

### 6.4.6.1 Gradient Descent Optimization Algorithm

Gradient descent is used as optimization technique to optimize the networks parameters. After each iteration, which passes one batch of data, gradient descent uses the loss to optimize he weights of the neural network. Its goal is to minimize the chosen loss function. Different gradient descent optimization algorithms are available, but the most popular optimization algorithms are Momentum, RMSProp and Adam. Adam can be seen as a combination of RMSprop and momentum, and is seen as the current overall best gradient descent optimization algorithm (Ruder, 2017). Adam adds bias-correction and momentum to RMSprop. Kingma and Ba (2015) show regardless of the hyperparameters Adam is equally good as or better than RMSprop. Therefore, we conclude that Adam (Adaptive Moment Estimation) is the most appropriate and will be used throughout this research project. As explained in Section 6.3, the available data is partioned into training, validation and test sets. Training of the autoencoders is done in a semisupervised manner. First, the autoencoder is unsupervised trained on normal data. During training for autoencoder (with Attention Mechanism) the mean squared error (MSE) is used as the loss function that the gradient descent optimization algorithm tries to minimize during training. For the variational autoencoder a custom loss function is used, which is composed of the MSE with the KL divergence loss.

$$M S E = \frac{1}{n} {\sum }_{i = i}^{n} {e}_{i}$$

where eᵢ is the difference between the actual sequence and the reconstructed sequence and n is the number of samples.

### 6.4.6.2 Learning Rate

The learning rate is a hyperparameter which controls how much, after each iteration, the weights of the neural network are adjusted. A low learning rate, results in small steps and requires more time to converge. Optimization might get stuck in a non local minima due to a too low learning rate. Contrary, a too high learning rate might result in too large steps which miss local minima. The Adam optimizer, as explained above, overcomes this issue by computing adaptive learning rates for each parameter after each iteration (Kingma & Ba, 2015). The optimizer uses the first moment (mean) and average of the second moment (variance) of the gradients to update the learning rate. Moreover, it uses an initial learning rate α, the exponential decay rate for the first moment estimates β₁, the exponential decay rate for the second-moment estimates β₂ and a very small number to prevent any division by zero in the implementation ϵ. Kingma and Ba (2015) propose α=0.001, β₁=0.9, β₂=0.999 and ϵ=10⁻⁸ as the default parameters. However, tuning the initial learning rate a could further improve the model performance (Brownlee, 2019; Mack, 2018).

### 6.4.6.3 Number of Units in Hidden Layer

The number of units in the hidden layers are related to over-fitting and under-fitting of a neural network. Under-fitting happens whenever a model fails to learn the problem and performs poorly on both the training set and test set. Over-fitting occurs whenever the training set is well learned, but performance is bad on the test set. Reducing the number of layers and number of units per layer prevent over-fitting.

### 6.4.6.4 Batch Size

Batch size is the final hyperparameter to be tuned. The batch size defines the number of samples to be propagated through the network at every iteration. As mentioned above, after each iteration the weights of the neural network are updated using the gradient descent optimization algorithm. Having a batch size equal to the number of samples is computational expensive as all samples are propagated through the network at once. It is generally known, training neural networks with a too large batch sizes is more sensitive to worse generalization compared to small batch sizes (Shirish Keskar, Mudigere, Nocedal, Smelyanskiy, & Tang, 2016). Training deep auto encoders with a small batch size generally also leads to solutions closer to the starting point than a large batch (Wang, Ren, & Song, 2017). Moreover, Shirish Keskar et al. (2016) indicate the batch size should be a value that is the power of 2. Following this logic the following range of batch values is used: 16, 32 or 64.

### 6.5 Deep Hybrid Anomaly Detection Models

The above described autoencoders are used to reconstruct a sequence and calculate the prediction error, then often a threshold is set which is used to determine whether a sequence is considered as an anomaly. However, at this stage the output of a deep autoencoder can also be used in a deep hybrid model. As explained in Chapter 2, deep hybrid models mainly utilize the autoencoders as feature extractors in order to feed traditional (unsupervised) anomaly detection algorithms (Nguyen et al., 2020). Nguyen et al. (2020) suggested to use the reconstruction error vector as input to one class SVM, whereas (Ghrib et al., 2020) utilized the latent space generated by the encoder as input to their supervised learning methods. Inspired by their approach, this research explores different types of autoencoders to capture non linear characteristics of multivariate data from the conching process and combines them with supervised classification methods to detect anomalous behaviour. Consequently, for each sequence both the error vector over time and the hidden space generated by different types of autoencoders are used as input to supervised learning methods. FIG. 20 illustrates an overview of the final architecture according to an embodiment. Supervised learning algorithms are used because for this study labels are available. However, choosing the appropriate supervised learning algorithm for the problem is not trivial, therefore different classification methods are examined based on the differences in the underlying techniques. These methods include a linear method, tree-based methods and a non-linear method. The remainder of this section briefly describes these methods.

### 6.5.1 Logistic Regression

Logistic regression is a linear method which models the relationship between the log odds of a dichotomous variable and a set of explanatory variables (D. Kleinbaum, Dietz, Gail, Klein, & Klein, 2002). The reconstruction error or latent variable are not necessary described in a linear fashion. However, linear regression is one of the most simple machine learning models and is known for its ease of interpretation (D. G. Kleinbaum & Klein, 2010). Therefore, the logistic regression serves as a base model within the deep hybrid anomaly detection methods. However, one disadvantage is its bad performance when multicollinearity or outliers in the data are present. The equation for logistic regression is shown in Equation 13. The model can easily be interpreting by looking at the βₙ coefficients. The coefficients βₙ of the logit model can be interpreted as the change in the log odds of an event when xₙ increases by one and all other variables are held constant. The coefficients can be transformed into odd ratios by calculating e to the power of βₙ (D. Kleinbaum et al., 2002).

$$log \frac{P ( y = 1 )}{1 - P ( y = 1 )} = {β}_{0} + {β}_{1} {x}_{1} + … + {β}_{n} {x}_{n}$$

### 6.5.2 Random Forest

A random forest is a bagging ensemble learning technique, which combines individual decision trees (Breiman, 1996). In order to reduce the bias of the model, every decision tree uses different samples of the data and different random subsets of features and makes its own prediction. Its main purpose is to add randomness and generate decorrelated decision trees (Garcia-Ceja et al., 2019). In the end, the class with the highest weighted average is predicted by the random forest. Another advantage of random forest includes the possibility to extract the feature importance within the forest (Garcia-Ceja et al., 2019). The feature importance could then be used as a feature selection tool prior. Utilizing the random forest within the deep hybrid anomaly detection model has some advantages. The supervised learning model is not prone to over-fitting, has good tolerance against outliers and noise, is not sensitive to multi-collinearity in the data and can handle data both in discrete and continuous form (Chen et al., 2020). Important hyperparameters include the maximum tree depth, the minimum samples for each split and the total number of trees. The maximum depth of a decision tree limits over fitting.

### 6.5.3 Boosting

Within boosting ensemble methods, different estimators are made sequentially which try to improve the previous estimation. It does this by building the ensemble incrementally and emphasizes the, by the previously model, incorrect classified training samples to train the next model. As such each training sample is assigned a weight which increases if the instance is miss-classified. In order to make a prediction in the end all model results are combined into a voting mechanism. Adaboost was one of the first boosting ensemble methods and was developed by Freund and Schapire (1997). Adaboost uses many weak algorithms (small decision trees known as stumps) to classify. As such, the number of trees is one of the most hyper parameters of adaboost trees, and the learning rate controls the contribution of each model to the ensemble prediction. However, boosting techniques can be very computational expensive. Gradient boosting technique can be utilized to overcome this issue (Friedman, 2001). Adaboost minimizes the exponential loss function, which can make the algorithm susceptible to outliers, whereas any differentiable loss function can be minimized with Gradient Boosting. Implying that for Adaboost the shortcomings are identified by high-weight data points, while gradient boosting uses the residuals of the previous models, also known as gradients. The residuals speed up the process because the weights do not have to be calculated. Important hyper parameters for gradient boosting trees include tree-specific parameters and the same boosting parameters as above. The tree specific parameters include the maximum depth and the minimum samples required for a split or leaf.

### 6.5.4 Support Vector Machine

Support Vector Machine (SVM) is a supervised learning algorithm which was originally introduced by Vapnik (1963). Originally, SVM was introduced to classify discrete multidimensional data. Further development also enabled to solve regression problems (Ay, Stemmler, Schwenzer, Abel, & Bergs, 2019). SVMs suitable for non-linear classification problems with small sample sizes, making it useful for anomaly detection (Wei, Feng, Hong, Qu, & Tan, 2017). SVM require an input vector which is then mapped with a nonlinear function and weighted with learned weights. The algorithm tries to find a decision boundary, known as a hyper-line, which linearly separates different examples of different categories or classes. SVM tries to maximize the perpendicular distance between the hyper-plane and the points closest to the hyper-plane, known as the support vectors. New cases which are to be predicted are mapped into this space, and based on their position in that space relative to that learned hyperplane the new cases are predicted (Vapnik, 1963). Contrary to most machine learning algorithms, Vapnik (1963) show that SVM minimizes the structural risk. Structural risk describes the over-fitting of the model and probability of misrepresenting untrained data (Ay et al., 2019). In case linear models cannot fit the data well, it is possible to apply computational expensive non-linear transformations of the features. Data is transformed into a higher dimensional space, for which the data is linearly separable. The kernel trick solves this problem by describing the data solely through pairwise similarity comparisons between observations. The data is then represented by these coordinates in the higher dimensional space, saving computational effort. Support vector machines have two main hyperparameters (C and gamma) which can be tuned to find the most suitable model for a problem. C represents the penalty of miss-classified data points. In case the radial basis function as kernel function in order to create a linear separable data set, gamma determines the actual influence of a single data point.

### 7 Chocolate Batch Anomaly Detection Results

Using the available process data, different deep learning and deep hybrid approaches have been selected. These methods are evaluated through experiments on the datasets, which include the selection of hyperparameters and their capability of detecting anomalous patterns. This chapter first explains the experimental set up, which lists the implementation details, evaluation metrics and the benchmark model. The benchmark model is used to compare the prediction performance of the anomaly detection models against the straightforward supervised classification approach. Afterwards, the development of the predictive models is explained. Section 7.2 explains how for each different autoencoder the hyperparameters are optimized. Besides, the section also visualizes the attention weight plots generated by the attention-based LSTM autoencoder. Once the autoencoders have learned the normal behaviour, Section 7.3 explains how the autoencoders can be utilized to detect undesired process behaviour. Section 7.4 explains how the output of the different autoencoders can serve as input to supervised models by generating semi-supervised deep hybrid models. A comparison of the performance between the traditional anomaly detection method (by setting a threshold) and deep hybrid models is given in Section 7.5. This section additionally inspects possible reasons for the miss-classifications. Based on the inspection, it is chosen to further investigate the use of different labels. For the out of control batches, the whole process is repeated and the results are shown in 7.6. The performances of both label types are compared in Section 7.7 and finally some concluding remarks are given in Section 7.8.

### 7.1 Experimental Setup

The different autoencoders and the benchmark deep classification model are implemented using Keras. Keras was developed by Chollet (2018) with the aim to enable fast experimentation. The supervised classification models within the deep hybrid approaches are fed with the output of the autoencoders. The supervised classification models and evaluation metrics were implemented using the scikit-learn library for Python, which was developed by Pedregosa et al. (2011). As an example, training and evaluating the models has been performed on a Processor Intel (R) Core (TM) i5-8365 CPU @ 1.60 GHz with 8 GB of RAM. Of course, use of a GPU could significantly decrease the training time of the neural networks. The quality of a prediction model will depend on how it is intended to use. Predictions of anomaly detection models are usually evaluated on its precision, recall and F8-score. Precision, shown in Formula 14, indicates how accurate the model is. It indicates out of positive predicted, how many of them are actual positive. While recall, shown in Formula 15, indicates the proportion of identified positives out of all actual positives. F8 score, shown in Formula 16, gives a measurement for the quality of a classifier by calculating a weighted fraction of recall and precision. It's a useful metric to consider when precision and recall are both important, but one requires some more attention than the other. From a business perspective, the model will be used as an alarming method in case a faulty batch occurs. As such, it is desired to minimize the number of false alarms and thus it is chosen to assign more importance to the precision, which is indicated by setting β=0.5.

$$Precision = \frac{TruePositive}{TruePositive + FalsePositive}$$

$$Recall = \frac{TruePositive}{TruePositive + FalsePositive}$$

$${F}_{β} = \frac{( 1 + {β}^{2} ) * Precision * Recall}{( {β}^{2} * Precision ) + Recall}$$

### 7.1.1 Benchmark Model

The current problem can be seen as a classification problem, for which the straightforward approach includes supervised learning to classify a fault. However, the class imbalance, as shown in Chapter 5, limited the modeling possibilities. As a result it was chosen to utilize (deep) autoencoders to learn exclusively from the majority class and perform anomaly detection. In order to validate the choice for the semi-supervised approach, the performance of the anomaly detection models is compared against a supervised binary classification model. Training the supervised classification models is different from training the autoencoders because classification requires both normal and anomalous labels. Therefore, for the benchmark model, the available data is partitioned into a train, validation and test split in a stratified fashion. Training benchmark models is performed using 70% of all data. Binary cross-entropy is used as the loss function which is minimized by the stochastic gradient descent. Hyperparameters, such as layers, number of neurons, learning rate and batch size are optimized using 15% of the validation data. Finally, the performance of the best performing benchmark model is evaluated on the remaining 15%, which is represented in the test set. This benchmark model consists of similar architecture as the encoder part of the normal autoencoder. As a result the benchmark model architecture consists of either one or two hidden layers to map the original data into a lower dimensional feature space. After compressing the data a dense layer with a single neuron and sigmoid activation is used to make binary predictions. An overview of the hyperparameters is shown in table 6.

TABLE 6
Hyperparameters of the supervised LSTM classifier, which is used
as benchmark model.

| | Values |
|---|---|
| RNN Units | LSTM |
| Gradient Descent Optimization Algorithm | Adam |
| Learning Rate | 0.0001, 0.00001 |
| Number of hidden layers | 1 or 2 |
| Number of Units | 32, 16, 8, 4 |
| Batch Size | 16, 32, 64 |

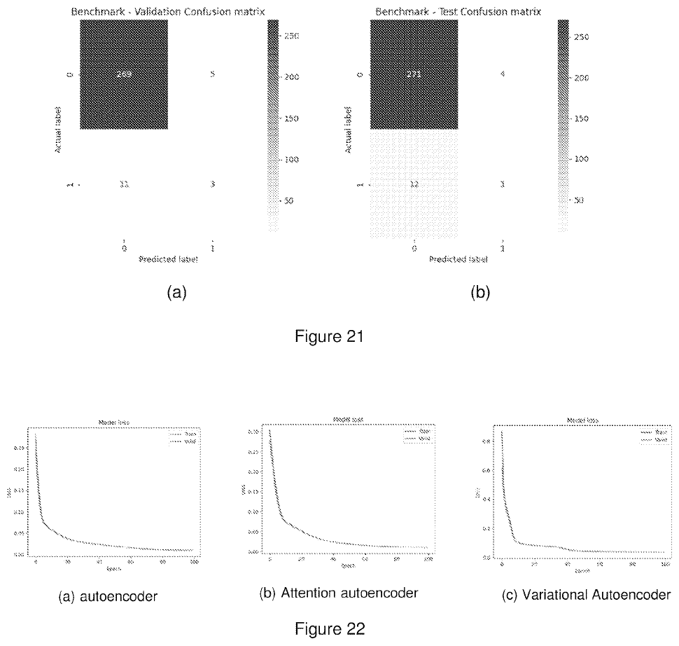

For sequences of 180 minutes the best performing model consists of only one hidden layer with 16 neurons, a learning rate of 0.0001 and a batch size of 64. The resulting validation and test confusion matrices are shown in FIG. 21. Although this architecture obtained highest validation performance, the precision, recall, F0.5 and F1 performance on the validation set are still low and equal respectively 37,5%, 21.4%, 32.6% and 27.7%. The model's performance on the test set is even worse. For the test set, the precision, recall, F0.5 and F1 equals 20%, 7.7%, 15.1% and 11.1%. The benchmark model seems to under-fit. The performance of the supervised classification models is as expected low which is caused due to the class imbalance. The remainder of this chapter explores whether training semi-supervised autoencoders are capable of achieving better performance.

### 7.2 Autoencoder Architecture and Parameter Tuning

As explained in Chapter 6, during this research three types of autoencoders are explored. These include a normal LSTM autoencoder, an LSTM autoencoder with multiplicative attention and a variational autoencoder. Additionally, within the normal autoencoders type, we explore whether utilizing more layers improves the results. The first autoencoder employment is the simplest LSTM autoencoder, with one input layer, one hidden encoder layer, one hidden decoder layer and one output layers. The number of hidden layers is fixed to two in this model type. Secondly, instead of using only one hidden layer for both the encoder and decoder, configurations with four hidden layers are checked. In the case of four hidden layers, the encoder and decoder both have two hidden layers. Finally, the model with the lowest reconstruction loss is chosen as the normal autoencoder. As explained in Section 6.4.4, Luong et al. (2015) suggests three different methods to calculate the alignment scores. For model simplicity, only one attention mechanism architecture with the dot function is chosen. Implementing the dot function requires only taking the dot product of the hidden states of the encoder and decoder. Moreover, again for simplicity only one variational autoencoder architecture is considered. The variational autoencoder is employed with one input layer, one hidden encoder layer. The encoder outputs a latent variable. The reparameterization trick is applied in the sampling layer, by sampling values and feed it into the decoder. Afterwards, the decoder decodes the sample back by reconstruction the input. As explained in Section 6.4.6, the hyperparameters of the autoencoders are optimized using grid-search. For each autoencoder type, the number of units in the hidden encoder and decoder layer is a tune-able hyperparameter. Additionally for the variational autoencoder the latent dimension is another hyperparameter. An overview of all hyperparameters is shown in Table 7. The autoencoders are trained on 70 percent of the normal samples which is known as the training set and validated exclusively on the normal samples present in the validation set.

TABLE 7
Overview of the Autoencoder Hyperparameters

| | Values |
|---|---|
| RNN Units | LSTM |
| autoencoder Type | AE, AE with Attention, VAE |
| Gradient Descent Optimization Algorithm | Adam |
| Learning Rate | 0.0001, 0.00001 |
| Number of Encoder layers | AE: 1 or 2, else 1 |
| Number of Decoder layers | AE: 1 or 2, else 1 |
| Number of Units | 32, 16, 8, 4 |
| Batch Size | 16, 32, 64 |

For each model type and multiple sequence lengths, the hyperparameters with the lowest loss is chosen as the best autoencoder, the hyperparameters and resulting loss are shown in Table 8. FIG. 22 shows the learning curves for the best normal autoencoder (22a), attention autoencoder (22b) and variational autoencoder (22c) for sequences of length 180 minutes. In all figures it can be observed the loss for both the train and validation set is low. Moreover, the training loss and validation loss are almost equal. Implying that the normal sequences in the validation data are well represented by the training data set and that the autoencoders are thus capable of learning the normal behaviour in the training and validation set. For the remainder of this study, the trained autoencoders using these parameters are used for detecting anomalies.

TABLE 8
Best Hyperparameters for Different Autoencoder Types trained on different
sequence lengths

| | | Encoder | | Decoder | | Learning | | |
|---|---|---|---|---|---|---|---|---|
| autoencoder | Sequence | Neurons | Neurons | Neurons | Neurons | | learning | End |
| Type | Length | 1 | 2 | 1 | 2 | batchsize | rate | Loss |
| Autoencoder | 180 | 16 | x | x | 16 | 16 | 0.0001 | 0.0095 |
| Autoencoder | 240 | 32 | 16 | 16 | 32 | 16 | 0.0001 | 0.0083 |
| Autoencoder | 300 | 32 | 16 | 16 | 32 | 32 | 0.0001 | 0.0121 |
| Attention | 180 | 16 | x | x | 16 | 32 | 0.0001 | 0.0492 |
| Attention | 240 | 16 | x | x | 16 | 16 | 0.0001 | 0.0356 |
| Attention | 300 | 8 | x | x | 8 | 16 | 0.0001 | 0.0446 |
| VAE | 180 | 32 | 16 | 16 | 32 | 16 | 0.0001 | 0.0360 |
| VAE | 240 | 32 | 16 | 16 | 32 | 16 | 0.0001 | 0.0356 |
| VAE | 300 | 16 | 8 | 8 | 16 | 16 | 0.0001 | 0.0393 |

### 7.2.1 Attention Weight Visualization

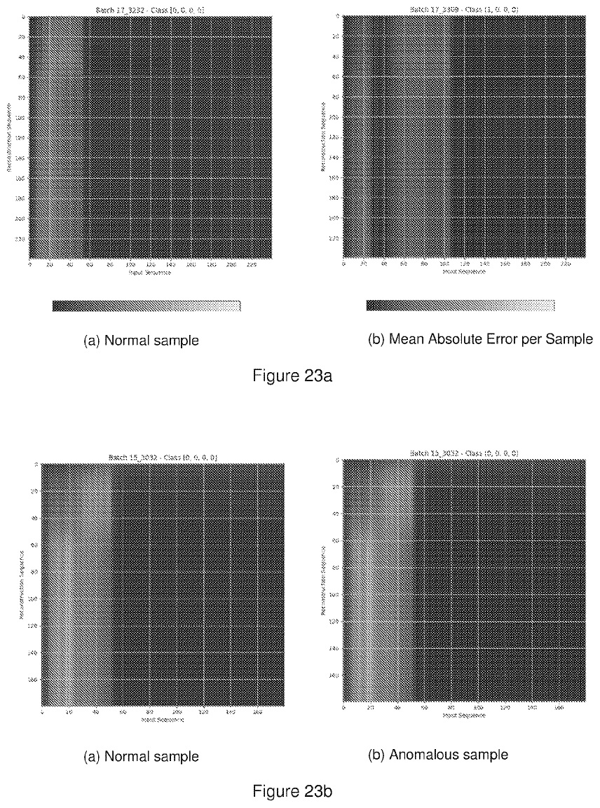

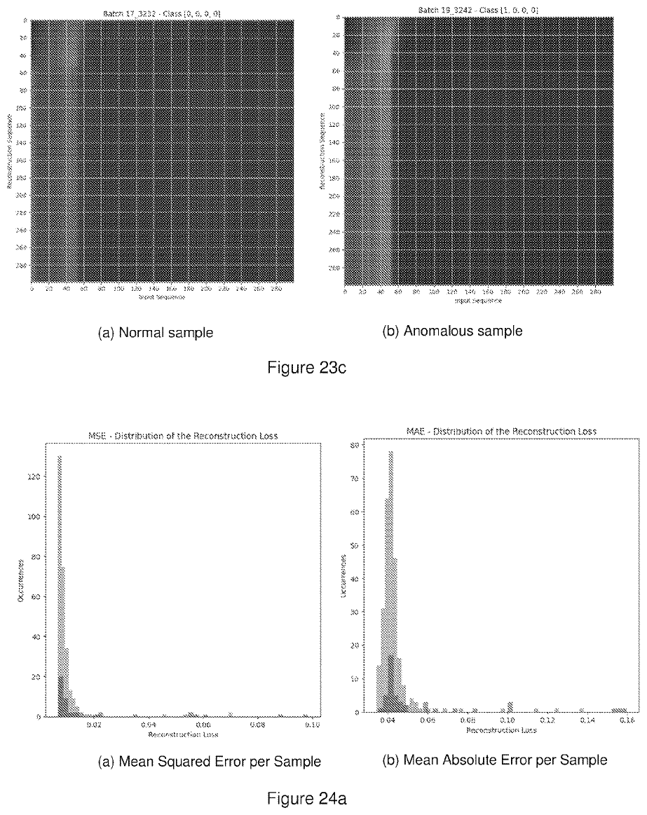

A major benefit of the attention mechanism is that it learns to pay more attention to certain encoded hidden states (Pereira & Silveira, 2019). As a result, the attention model produces a 2D map for each sequence with length T which visualizes where the neural network is putting its attention. As an example, FIG. 23 shows the attention maps for one normal and one anomalous sample for sequences of length 240. The map shows which input minutes, shown on the x-axis, are considered as most important for the reconstruction on the y-axis.

FIG. 23a illustrates the autoencoder assigns most attention weights to the first 50 minutes for the normal sample. Earlier data exploration, in Chapter 5, already highlighted this is the time it takes until the conche is filled with all the required raw materials. A similar observation can be made for the anomalous sample which is shown in FIG. 23b. The figure clearly indicates the autoencoder assigns more weights to the first 100 minutes. In addition, the model stresses an attention gap around the usual 40-50 minutes. Deeper data exploration for this specific case, reveals that the filling phase for this sample ends at 105 minutes. The attention mechanism mainly assigns attention to the first 60 minutes of the sequence. As a result, the attention maps show evidence for the intuition that the filling phase is of much importance for the final quality of a chocolate batch. Moreover, these plots show evidence that the autoencoder with attention mechanism can produce context-aware representations (Pereira & Silveira, 2019).

### 7.3 Autoencoder Anomaly Detection Performance

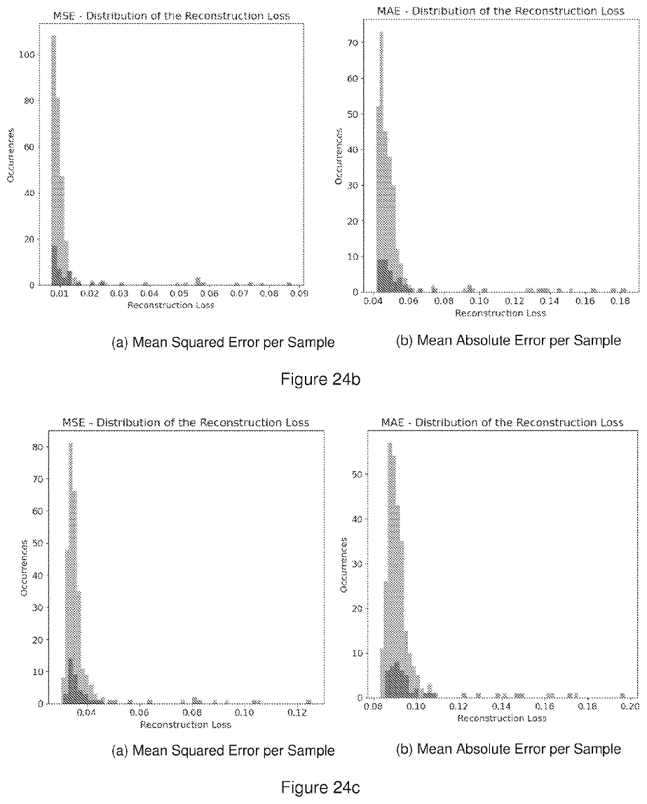

This section describes how the representations of the normal behaviour learned by the autoencoders can be utilized to detect undesired process behaviour. At first, the distribution of the reconstruction losses is explored. The reconstruction loss is computed by subtracting the reconstructed sequence from the original input. FIG. 24 shows the distribution of the MSE and MAE generated by the normal autoencoder for samples of length 180 minutes in the validation set. The figure illustrates the MSE and MAE and the normal chocolate batches are illustrated in green, whereas anomalous batches are shown in red. It can immediately be observed the two classes overlap and cannot easily be separated. As a result setting a threshold to detect anomalous samples, which is further explained in Section 7.3.1, is not expected to provide good results. When comparing the distributions of MSE (FIG. 24a) and MAE (FIG. 24b), we observe in case of relatively larger errors the MSE has slightly less green observations. Implying that it should be easier to set a threshold using the MSE value. For both autoencoder types, both the MSE and MAE reconstruction loss distributions look quite similar compared to the normal AE. Therefore, for the remainder of this study the MSE values are used to set a threshold.

### 7.3.1 Detecting Anomalies by Setting a Threshold

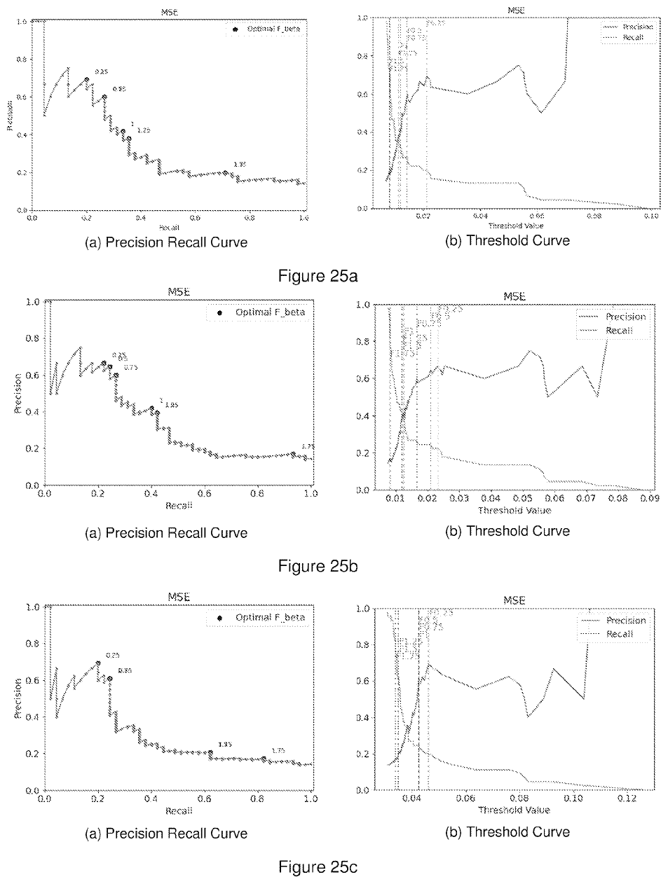

As explained in Chapter 6, the reconstruction error can be used to detect anomalies. The MSE of the samples in the validation set is used to determine the actual threshold. The whole process is explained using the normal AE model trained on 180 minute sequences, but is similar for all other autoencoder types. FIG. 25a illustrates the trade off for the precision and recall curve of the AE model trained on 180 minutes. As explained in Section 7.1, the F_{β} is a single score which evaluates both the precision and recall. Using the Fa scores to move the threshold counters the need for manual selection. Each threshold achieves a certain precision and recall score on the validation set, which can be used to determine the F_{β} score. Then for a given β, the threshold with the highest F_{β} score is considered as the optimal threshold for that β value. In order to prevent over-fitting, selecting the final threshold should be based on the validation set. Therefore, the F_{β} scores and the corresponding threshold values are calculated using the validation set. The performances of the chosen threshold can eventually be compared using the unseen test set.

FIG. 25a indicates the optimal points for a chosen F_{β}. The figure clearly demonstrates β<1 assigns more weight to precision, whereas a β>1 assigns more weight to recall.

For all different F-scores, FIG. 25b shows their corresponding thresholds with its validation precision and recall performance. The Figures show, even though different beta scores are used, still the same threshold value can be chosen. During the data exploration, it was already shown that the normal and anomalous chocolate batches have quite similar patterns. As shown in the previous section, training the autoencoder models purely on the normal instances will result in reconstruction loss distributions which have much overlap. As a consequence, it is quite hard to detect anomalous samples and performance is expected to be low. Additionally, the practical implementation concerns alarming whenever an incorrect chocolate batch occurs. It is thus desired to minimize the number of false alarms and therefor it is chosen to assign more importance to the precision. Implying, that only the thresholds for F0.25, F0.5 and F0.75 are considered as final threshold options. The other thresholds are also shown because it provides much indication what happens when changing the beta value.

Table 9 shows the performance of each threshold on the test set. Here it can be observed the F0.25 obtains best performance. The F0.25 is the only threshold which obtains similar test and validation performances and is thus not over-fitting. It has a relatively high test precision of 75 percent, but low recall of 20 percent, which was similar to the validation set. The F0.5 and F0.75 scores have the same threshold value, and thus share same test performance. It can be observed their test performance drops as the validation precision was 60% and is now 50%, while the recall stays quite similar around 24%.

TABLE 9
Test Performance of chosen thresholds for the normal Autoencoder. The
thresholds are determined using the Fbeta scores using the validation set.
In order to minimize false alarms, the threshold with the highest F0.5
score is considered as the best threshold

| | Threshold | Test Performance | | | | | | |
|---|---|---|---|---|---|---|---|---|
| Fbeta | Value | TN | FP | FN | TP | Precision | Recall | F0.5 |
| F0.25 | 0.02133 | 271 | 3 | 37 | 9 | 0.750 | 0.196 | 0.48 |
| F0.5 | 0.01443 | 263 | 11 | 35 | 11 | 0.500 | 0.239 | 0.41 |
| F0.75 | 0.01443 | 263 | 11 | 35 | 11 | 0.500 | 0.239 | 0.41 |
| F1 | 0.01196 | 251 | 23 | 32 | 14 | 0.378 | 0.304 | 0.36 |
| F1.25 | 0.01136 | 246 | 28 | 32 | 14 | 0.333 | 0.304 | 0.33 |
| F1.5 | 0.00826 | 136 | 138 | 17 | 29 | 0.174 | 0.630 | 0.20 |
| F1.75 | 0.00826 | 136 | 138 | 17 | 29 | 0.174 | 0.630 | 0.20 |

Same approach is performed for the other two autoencoder types. For the attention autoencoder, during validation, the trade-off between the optimal F0.25 and F0.5 seems to be quite similar. The first has a slightly higher precision, whereas the second has a slightly higher recall. Table 10 shows the final threshold performance on the test set. For the attention autoencoder, the best performance is also obtained using the F0.25 threshold. Both thresholds detect 10 anomalies, where the F0.5 has one more false alarm. Comparing the attention autoencoder with the normal autoencoder, it can be observed the attention model is capable of detecting one additional anomaly with the same amount of false positives.

TABLE 10
Test Performance of chosen thresholds for the Attention-based Autoencoder.
The thresholds are determined using the Fbeta scores using the
validation set. In order to minimize false alarms, the threshold
with the highest F0.5 score is considered as the best threshold

| | Threshold | Test Performance | | | | | | |
|---|---|---|---|---|---|---|---|---|
| Fbeta | Value | TN | FP | FN | TP | Precision | Recall | F0.5 |
| F0.25 | 0.0232 | 271 | 3 | 36 | 10 | 0.769 | 0.217 | 0.510 |
| F0.5 | 0.0210 | 270 | 4 | 36 | 10 | 0.714 | 0.217 | 0.490 |
| F0.75 | 0.0166 | 261 | 13 | 35 | 11 | 0.458 | 0.239 | 0.387 |
| F1 | 0.0125 | 250 | 24 | 30 | 16 | 0.400 | 0.348 | 0.388 |

For the last autoencoder type, the chosen threshold and their test performance is given below in Table 11. The best performance is expected for the F0.25 threshold. The corresponding F0.5 and F0.75 threshold share the same value as the F1 threshold, implying that these do not favour the precision score. Table 11 shows the F0.25 threshold is the optimal threshold which is capable of detects most anomalies. Although this autoencoder type detects the most anomalies, it provides 7 false positives.

TABLE 11
Test Performance of chosen thresholds for the Variational Autoencoder. The
thresholds are determined using the Fbeta scores using the validation set.
In order to minimize false alarms, the threshold with the highest F0.5
score is considered as the best threshold

| | Threshold | Test Performance | | | | | | |
|---|---|---|---|---|---|---|---|---|
| Fbeta | Value | TN | FP | FN | TP | Precision | Recall | F0.5 |
| F0.25 | 0.0462 | 267 | 7 | 35 | 11 | 0.611 | 0.239 | 0.466 |
| F0.5 | 0.0425 | 264 | 10 | 35 | 11 | 0.524 | 0.239 | 0.423 |
| F0.75 | 0.0425 | 264 | 10 | 35 | 11 | 0.524 | 0.239 | 0.423 |
| F1 | 0.0425 | 264 | 10 | 35 | 11 | 0.524 | 0.239 | 0.423 |

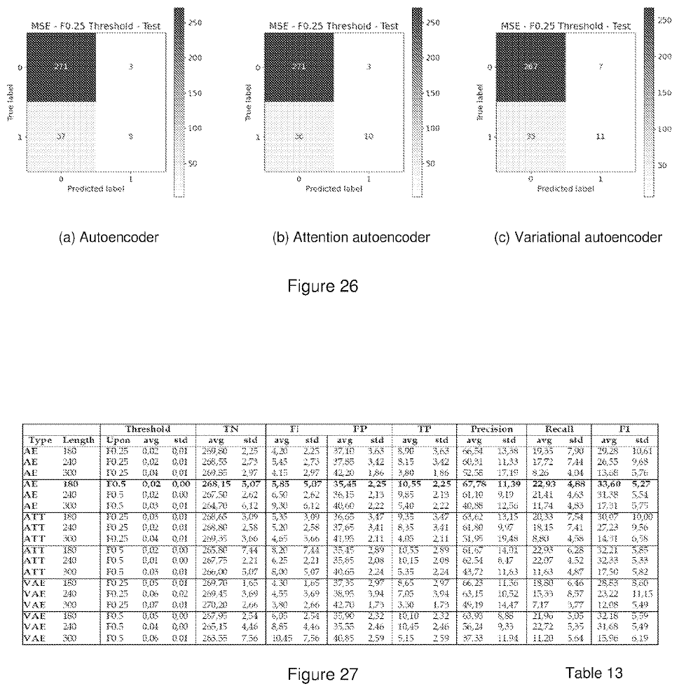

The confusion matrices for the optimal thresholds are shown in FIG. 26 and an overview of the final test performance of different autoencoders with MSE Thresholding is shown in Table 12. For all autoencoder types the F0.25 obtained best performance in terms of trade off between precision and recall (as indicated by the highest F0.5 performance). Comparing the different autoencoder types, it seems utilizing attention yields improves the performance. It is capable of detecting 10 anomalies, while producing only three false alarms. Moreover, the confusion matrices show the VAE model is capable of detecting the most anomalies, but at a cost of more false positives. Although the performances of the autoencoder anomaly detection models are not high, the results still validate the choice to prefer semi-supervised approach over the supervised approach. Table 12 shows each the obtained FB performance of semi-supervised approach is at least three times higher compared to the supervised benchmark.

TABLE 12
Overview autoencoders Test Performance. In order to minimize
false alarms, most importance is assigned to precision indicated
by a high F0.5 score

| Autoencoder | Threshold | Precision | Recall | F0.5 |
|---|---|---|---|---|
| Autoencoder | 0.0213 | 75.00% | 19.57% | 47.87% |
| Attention Autoencoder | 0.0232 | 76.92% | 21.74% | 51.04% |
| Variational Autoencoder | 0.0462 | 61.11% | 23.92% | 46 .−1% |

| | | | |
|---|---|---|---|
| Benchmark-LSTM Classifier | 20.00% | 7.69% | 15.15% |

### 7.3.2 Validation and Test Split Sensitivity Analysis

As mentioned in Section 6.3, 70 percent of the normal data is randomly split and used for training the autoencoders. In order to prevent data leakage this data was discarded. The other 30 percent and all anomalous data is also randomly split into a validation and test set. This section explores whether the randomly chosen validation and test set splits influences the performance. For this analysis, the average and standard deviation of the true negatives, false positives, false negatives, true positives, precision, recall and F1 score over 20 different validation and test splits are obtained. Instead of manual setting and in order to have a fair comparison again different F_{β} thresholds are used. FIG. 27 provides an overview of all most important results, including insights in the effect of having different validation and test splits. For each model, the threshold is determined using the FB value on the validation set

In the previous section, the F0.25 threshold was chosen as best threshold for all models for a single validation and test split. Contrary, in case of the sensitivity analysis for the normal autoencoder the F0.5 threshold is favoured for sequences of length 180 and 240 minutes. For both sequence lengths this threshold value yields an average higher test precision and recall accompanied with a lower standard deviation. However, it must be mentioned the differences are quite small. For sequences of length 300 minutes, the F0.25 does yield an average higher precision compared to the F0.5 threshold, but it must be mentioned this precision is still extremely low. For the attention autoencoder, the performance of both thresholds shows little differences. For sequences of length 180 the F0.25 threshold has on average a slightly higher precision with a slightly lower standard deviation, but its recall performance shows opposite behaviour. As a result, on average the less recall penalizing F0.5 threshold seems to be better. For the length 240 minutes, the F0.50 threshold is again better as it obtains similar precision, but slightly higher recall. Similar as with the normal autoencoder, the attention autoencoder for sequences of length 300 minutes obtains bad performance. For this sequence length the F0.25 yields a higher precision, but this precision is again very low. Similar findings are observed for the VAE model, where again the F0.50 threshold seems to have the best overall performance compared to the F0.25. For the VAE autoencoder on all sequence lengths the F0.50 threshold achieves a higher F1 score compared to the F0.25 score. Overall, the F0.50 threshold is capable of obtaining a better average weighted trade off between precision and recall on all sequence lengths.

Further, for all autoencoders types we observe similar behaviour; on average the F0.50 threshold shows the best average trade-off between precision and recall and its performance decreases if the sequence length becomes longer. Implying that extending the sequences with more minutes, does only induce more noise and does not enable easier anomaly detection. Therefore, for the remainder of this study only sequences of length 180 minutes are used. Moreover, the differences between the normal, attention and variational autoencoder using the F0.50 threshold seem to be quite small. The normal autoencoder achieves on average the highest precision, recall and F1 score. As a consequence, this autoencoder is considered as the best performing anomaly detection threshold model. Additionally, we compare the performances of the autoencoder with and without attention mechanisms. We observe that the performance of the attention autoencoders becomes higher than the normal autoencoder if the sequence length is increased. In case of the F0.25 threshold, on average the attention autoencoder scores better in terms of F1 score for all sequence lengths. As stated above, the overall best performing model is the F0.50 normal autoencoder for sequence length 180. This model has a higher precision and equal recall compared to the attention autoencoder. However, if the sequence length is increased to 240 or 300 minutes, the F0.50 attention autoencoder obtains higher precision, recall and F1 scores, indicating that the attention mechanism is beneficial for longer sequences.

### 7.4 Detecting Anomalies Using Deep Hybrid Models

In the previous two sections it may be observed, poor results are obtained for anomaly detection models which only utilize setting a threshold on the reconstruction error. This section explores whether deep hybrid models improve the prediction capabilities. As described in Chapter 6, the output of an autoencoder can serve as input to another machine learning model, which is known as a deep hybrid model. Nguyen et al. (2020) used the reconstruction error vector as input, while Ghrib et al. (2020) use the output of the encoder of a fully trained autoencoder, known as the latent space. Consequently, for each sequence of 180 minutes both the error vector over time and the latent space generated by different types of autoencoders are explored. First, hyperparameter tuning for each of the supervised models is performed. In order to prevent data leakage, after training the autoencoders the training set is discarded. The autoencoder learned this set of data as normal behaviour and as a result it is likely this set has misleading reconstructions or a misleading latent space. Due to the removal of this sample set only a small set of 639 samples remains available for optimizing the supervised algorithms. Tuning the hyperparameters, which are shown in Table 14, is performed using grid-search on the validation set. As the dataset is quite small grid-search is combined with Repeated Stratified Kfold, which divides the validation set into three different folds and iterates three times over these folds to retrieve the optimal hyperparameters for each model. Stratified folding was used to ensure each fold consists of anomalous and normal samples. For consistency, for each deep hybrid model combination, the parameters with the highest F0.5 cross-validation performance is chosen as the final model configuration. Once the final hyperparameters are found and as the validation set is already very small, training is again performed on the full validation set because training the model on more data makes it more likely to generalize to unseen data.

TABLE 14
Overview of tuned parameters for each classifier

| Classifier | Parameter | Hyper Parameter Ranges |
|---|---|---|
| Random Forest | n estimators | 100, 200, 500, 1000 |
| | max features | auto, log2 |
| | max depth | None, 10, 50, 100 |
| | min samples split | 2, 5, 10 |
| | min samples leaf | 1, 3, 4 |
| | bootstrap | True, False |
| LogisticRegression | solver | newton-cg, lbfgs, liblinear |
| | C | 0.01, 0.1, 1, 10, 100 |
| | Penalty | L2 |
| AdaBoost | learning rate | 0.01, 0.1, 1 |
| | n estimators | 100, 200, 500, 1000 |
| GradientBoosting | learning rate | 0.01, 0.1, 1 |
| | n estimators | 100, 200, 500, 1000 |
| | min samples split | 2, 5, 10 |
| | min samples leaf | 1, 3, 4 |
| | max depth | None, 10, 50, 100 |
| SVC | kernel | rbf |
| | gamma | scale, 0.001, 0.01, 0.1, 1, 10 |
| | C | 0.1, 1, 10, 100, 1000 |

### 7.4.1 Reconstruction Error Per Minute

This section provides the results of using mean squared error per minute vector generated by the different autoencoders as input to different supervised learning algorithms. The explored approach is inspired by Nguyen et al. (2020), which used the reconstruction error vector as input to one class support vector machines. The cross-validation results of the hyperparameter tuning are listed in Table 15, and its corresponding best parameters are given in Table 15b. The Normal AE combined with SVC shows highest average validation precision which equals 76.39%, but its recall is too low. It can be observed the for each autoencoder type, the hybrid combination with the random forest has highest crossvalidation F0.5 performance. When comparing all three autoencoder types, the attention autoencoder scores best with a cross-validation F0.5 equaling 47.72%. The performance of the hybrid combination using the normal and variational autoencoders score about equal because the F0.5 values equal respectively 41.25% and 40.85%.

TABLE 15
Cross-validation Hyperparameter Tuning Results Reconstruction error per
minute as input to supervised models. In order to minimize false alarms, most importance
is assigned to precision indicated by a high F0.5 score

| | | Precision | | Recall | | F0.5 | | F1 | |
|---|---|---|---|---|---|---|---|---|---|
| Type | Classifier | Avg | Std | Avg | Std | Avg | Std | Avg | Std |
| AE | AdaBoost | 44.99 | 12.48 | 21.11 | 7.11 | 36.11 | 10.40 | 28.33 | 8.78 |
| AE | Gradient Boosting | 63.90 | 18.92 | 20.00 | 7.70 | 39.99 | 7.91 | 28.60 | 8.59 |
| AE | Logistic | 53.47 | 24.11 | 18.89 | 13.01 | 33.53 | 12.93 | 25.17 | 13.76 |
| | Regression | | | | | | | | |
| AE | Random Forest | 62.78 | 21.98 | 18.89 | 5.98 | 41.25 | 10.70 | 28.37 | 8.10 |
| AE | SVC | 76.39 | 26.53 | 13.33 | 5.44 | 38.53 | 13.65 | 22.47 | 8.63 |
| ATT | AdaBoost | 45.48 | 12.16 | 21.11 | 8.09 | 34.61 | 6.18 | 27.12 | 6.72 |
| ATT | Gradient Boosting | 67.46 | 16.76 | 20.00 | 6.67 | 44.12 | 9.05 | 30.06 | 7.91 |
| ATT | Logistic | 35.42 | 12.21 | 12.22 | 4.58 | 24.28 | 6.72 | 17.48 | 5.56 |
| | Regression | | | | | | | | |
| ATT | Random Forest | 66.31 | 16.23 | 25.56 | 11.17 | 47.72 | 11.49 | 35.26 | 11.42 |
| ATT | SVC | 56.44 | 22.59 | 22.22 | 13.70 | 40.45 | 17.73 | 30.27 | 15.55 |
| VAE | AdaBoost | 44.31 | 22.93 | 20.00 | 10.18 | 34.24 | 17.85 | 26.45 | 13.48 |
| VAE | Gradient Boosting | 58.24 | 36.85 | 17.78 | 9.16 | 37.14 | 19.99 | 25.76 | 13.10 |
| VAE | Logistic | 33.06 | 19.73 | 14.44 | 9.75 | 25.51 | 15.81 | 19.61 | 12.63 |
| | Regression | | | | | | | | |
| VAE | Random Forest | 62.70 | 22.77 | 17.78 | 3.14 | 40.85 | 10.57 | 27.31 | 5.48 |
| VAE | SVC | 49.80 | 15.45 | 18.89 | 11.81 | 35.84 | 14.57 | 26.36 | 13.55 |

Table 15b: Reconstruction Error per Minute-Optimal Hyper-parameters hybrid models

TABLE 15b
Reconstruction Error per Minute-Optimal Hyper-parameters
hybrid models

| AE | CLF | params |
|---|---|---|
| AB | AdaBoost | {‘learning_rate’: 1, ‘n_estimators’: 500} |
| AE | GradientBoosting | {‘learning_rate’: 1, ‘max_depth’: 10, |
| | | ‘min_samples_leaf’: 3, |
| | | ‘min_samples_split’: 10, ‘n_estimators’: 200} |
| AE | LogisticRegression | {‘C’: 1000, ‘penalty’: ‘l2’, ‘solver’: ‘liblinear’} |
| AE | RandomForest | {‘bootstrap’: True, ‘max_depth’: 10, ‘max_ |
| | | features’: ‘auto’, ‘min_samples_leaf’: 1, |
| | | ‘min_ samples_split’:7, ‘n_estimators’: 100} |
| AE | SVC | {‘C’: 10, ‘gamma’: 1, ‘kernel’: ‘rbf’} |
| ATT | AdaBoost | {‘learning_rate’: 1, ‘n_estimators’: 1000} |
| ATT | GradientBoosting | {‘learning_rate’: 1, ‘max_depth’: 50, |
| | | ‘min_samples_leaf’: 4, |
| | | ‘min_samples_split’: 5, ‘n_estimators’: 200} |
| ATT | LogisticRegression | {‘C’: 1000, ‘penalty’: ‘l2’, ‘solver’: ‘liblinear’} |
| ATT | RandomForest | {‘bootstrap’: False, ‘max_depth’: None, ‘max_ |
| | | features’: ‘auto’, ‘min_samples_leaf’: 1, ‘min_ |
| | | samples_split’: 10, ‘n_estimators’: 100} |
| ATT | SVC | {‘C’: 10, ‘gamma’: ‘scale’, ‘kernel’: ‘rbf’} |
| VAE | AdaBoost | {‘learning_rate’: 0.1, ‘n_estimators’: 500} |
| VAE | GradientBoosting | {‘learning_rate’: 1, ‘max_depth’: 10, |
| | | ‘min_samples_leaf’: 1, |
| | | ‘min_samples_split’: 10, ‘n_estimators’: 100} |
| VAE | LogisticRegression | (‘C’: 1000, ‘penalty’: ‘l2’, ‘solver’: ‘liblinear’} |
| VAE | RandomForest | {‘bootstrap’: True, ‘max_depth’: None, ‘max_ |
| | | features’: ‘auto’, ‘min_samples_leaf’: 1, ‘min_ |
| | | samples_split’: 5,‘n_estimators’: 100} |
| VAE | SVC | {‘C’: 10, ‘gamma’: 10, ‘kernel’: ‘rbf’} |

The final performance of the deep hybrid which utilized the reconstruction error per minute autoencoder output on the test set is given in Table 16. It can immediately be observed that each deep hybrid model obtains higher performance compared to the benchmark model. This again validates the chosen semi supervised approach over the supervised approach. Similar to the cross-validation results, the best F0.5 performance is obtained for the attention autoencoder combined with the random forest. However, the normal autoencoder with the random forest and the variational autoencoder combined with an SVC score a slightly lower but about equal performance. Within each autoencoder type, these three hybrid combinations outperform the others. However, across the autoencoder types the performance is about equal as the differences only include one additional true or false positive. Because differences across these models are this small and Section 7.3.2 earlier demonstrated the high variability due to the chosen splits, in the next section again a sensitivity analysis is performed to account for the effect of having different validation test splits. For the sensitivity analysis, the performance of the hyperparameters is checked using different train test splits. Compared to the threshold methods, the hybrid models have one additional advantage. The hybrid models using the mean squared error per minute facilitate direct interpretable models which will be explained in Section 8.1.

TABLE 16
Test Performance Reconstruction error per minute as input to supervised
models In order to minimize false alarms, most importance is assigned to precision indicated
by a high F0.5 score.

| autoencoder | Classifier | TN | FP | FN | TP | Precision | Recall | F0.5 | F1 |
|---|---|---|---|---|---|---|---|---|---|
| AE | AdaBoost | 273 | 1 | 41 | 5 | 83.33 | 10.87 | 35.71 | 19.23 |
| AE | Gradient Boosting | 269 | 5 | 39 | 7 | 58.33 | 15.22 | 37.23 | 24.14 |
| AE | Logistic | 273 | 1 | 43 | 3 | 75.00 | 6.52 | 24.19 | 12.00 |
| | Regression | | | | | | | | |
| AE | Random Forest | 271 | 3 | 37 | 9 | 75.00 | 19.57 | 47.87 | 31.03 |
| AE | SVC | 271 | 3 | 43 | 3 | 50.00 | 6.52 | 21.43 | 11.54 |
| ATT | AdaBoost | 272 | 2 | 40 | 6 | 75.00 | 13.04 | 38.46 | 22.22 |
| ATT | Gradient Boosting | 270 | 4 | 37 | 9 | 69.23 | 19.57 | 45.92 | 30.51 |
| ATT | Logistic | 273 | 1 | 42 | 4 | 80.00 | 8.70 | 30.30 | 15.69 |
| | Regression | | | | | | | | |
| ATT | Random Forest | 270 | 4 | 36 | 10 | 71.43 | 21.74 | 49.02 | 33.33 |
| ATT | SVC | 268 | 6 | 37 | 9 | 60.00 | 19.57 | 42.45 | 29.51 |
| VAE | AdaBoost | 269 | 5 | 41 | 5 | 50.00 | 10.87 | 29.07 | 17.86 |
| VAE | Gradient Boosting | 267 | 7 | 41 | 5 | 41.67 | 10.87 | 26.60 | 17.24 |
| VAE | Logistic | 273 | 1 | 43 | 3 | 75.00 | 6.52 | 24.19 | 12.00 |
| | Regression | | | | | | | | |
| VAE | Random Forest | 268 | 6 | 37 | 9 | 60.00 | 19.57 | 42.45 | 29.51 |
| VAE | SVC | 271 | 3 | 37 | 9 | 75.00 | 19.57 | 47.87 | 31.03 |

| | | | | | | | | |
|---|---|---|---|---|---|---|---|---|
| Benchmark: LSTM Classifier | 271 | 4 | 12 | 1 | 20.00 | 7.69 | 15.15 | 11.11 |

### 7.4.2 Latent Output of Encoder

Contrary to the section above, which utilized the full autoencoder, this section investigates the performance when only the encoder part is used. Inspired by Ghrib et al. (2020), this section uses the encoder output of a fully trained autoencoder as input to different supervised learning classification algorithms. All results are shown in tables 17a and 17b, where first the hyperparameter optimization results are given and then the performance on the test set is evaluated. Table 17b lists the cross-validation performance for the best performing model, whereas Table 17c lists the corresponding parameters for the latent vectors produced by different autoencoder types. In case the latent vectors are used as input, the highest average precision is obtained for the normal AE combined with Logistic Regression with an average precision of 70.56%, however the recall and consequently the F0.5 is too low. The normal autoencoder obtains highest average F0.5 performance when combining it with an SVM. Similar for the attention autoencoder, and even across the autoencoder types the highest average performance is also obtained by combining the attention autoencoder with an SVM. The validation performance of the deep hybrid models which use the VAE encoder, seem to have extremely low performance indicating these are under-fitting.

TABLE 17a
Reconstruction Er-or per Minute-Optimal Hyper-parameters hybrid models

| | | Precision | | Recall | | F0.5 | | F1 | |
|---|---|---|---|---|---|---|---|---|---|
| Type | Classifier | Avg | Std | Avg | Std | Avg | Std | Avg | Std |
| AE | AdaBoost | 40.32 | 13.99 | 24.44 | 11.33 | 35.11 | 13.61 | 29.90 | 12.73 |
| AE | Gradient Boosting | 49.81 | 26.84 | 26.67 | 9.43 | 41.32 | 18.36 | 33.77 | 12.76 |
| AE | Logistic Regression | 70.56 | 24.98 | 14.44 | 8.09 | 37.02 | 15.67 | 23.12 | 11.71 |
| AE | Random Forest | 57.26 | 16.84 | 21.11 | 9.75 | 41.23 | 12.67 | 30.01 | 11.32 |
| AE | SVC | 64.38 | 22.34 | 24.44 | 6.29 | 47.53 | 13.07 | 34.84 | 8.71 |
| ATT | AdaBoost | 44.59 | 11.11 | 27.78 | 8.09 | 39.01 | 9.67 | 33.53 | 8.83 |
| ATT | Gradient Boosting | 67.30 | 25.60 | 17.78 | 4.97 | 39.37 | 6.29 | 26.37 | 4.89 |
| ATT | Logistic Regression | 62.10 | 26.07 | 17.78 | 11.33 | 38.86 | 17.35 | 26.33 | 13.92 |
| ATT | Random Forest | 60.20 | 23.12 | 16.67 | 3.33 | 38.11 | 10.19 | 25.41 | 5.35 |
| ATT | SVC | 68.92 | 20.88 | 30.00 | 9.23 | 54.20 | 15.04 | 41.43 | 11.61 |
| VAE | AdaBoost | 17.47 | 12.31 | 12.22 | 7.11 | 15.92 | 10.70 | 14.18 | 8.95 |
| VAE | Gradient Boosting | 36.11 | 27.18 | 14.44 | 11.81 | 26.77 | 20.00 | 20.06 | 15.63 |
| VAE | Logistic Regression | 16.11 | 13.93 | 4.44 | 3.14 | 10.07 | 7.79 | 6.70 | 4.85 |
| VAE | Random Forest | 26.98 | 20.65 | 7.78 | 5.98 | 17.15 | 12.56 | 11.67 | 8.73 |
| VAE | SVC | 33.33 | 10.67 | 8.89 | 3.14 | 20.90 | 5.88 | 13.80 | 4.41 |

TABLE 17b
Latent Input-Optimal Hyperparameters-hybrid models-Cross Validation Results

| AE | CLF | params |
|---|---|---|
| AE | AdaBoost | {‘learning_rate’: 0.1, ‘n_estimators’: 1000} |
| AE | GradientBoosting | {‘learning_rate’: 0.1, ‘max_depth’: None, ‘min_samples_leaf’: |
| | | 3, ‘min_samples_split’: 7, ‘n_estimators’: 500} |
| AE | LogisticRegression | {‘C’: 1000, ‘penalty’: ‘l2’, ‘solver’: ‘lbfgs’} |
| AE | RandomForest | {‘bootstrap’: False, ‘max_depth’: 10, ‘max_features’: |
| | | ‘log2’, ‘min_samples_leaf’: 3, ‘min_samples_split’: 2, ‘n_ |
| | | estimators’: 100} |
| AE | SVC | {‘C’: 1000, ‘gamma’: 1, ‘kernel’: ‘rbf’} |
| ATT | AdaBoost | {‘learning_rate’: 1, ‘n_estimators’: 1000} |
| ATT | GradientBoosting | (‘learning_rate’: 1, ‘max_depth’: 100, |
| | | ‘min_samples_leaf’: 1, |
| | | ‘min_samples_split’: 7, ‘n_estimators’: 100} |
| ATT | LogisticRegression | {‘C’: 1000, ‘penalty’: ‘l2’, ‘solver’: ‘liblinear’} |
| ATT | RandomForest | {‘bootstrap’: False, ‘max_depth’: 100, ‘max_features’: |
| | | ‘auto’, ‘min_samples_leaf’: 3, ‘min_samples_split’: 10, ‘n_ |
| | | estimators’: 200} |
| ATT | SVC | {‘C’: 1000, ‘gamma’: 1, ‘kernel’: ‘rbf’} |
| VAE | AdaBoost | {‘learning_rate’: 1, ‘n_estimators’: 1000} |
| VAE | GradientBoosting | {‘learning_rate’: 0.1, ‘max_depth’: 100, |
| | | ‘min_samples_leaf’: 3, |
| | | ‘min_samples_split’: 10, ‘n_estimators’: 500} |
| VAE | LogisticRegression | {‘C’: 10, ‘penalty’: ‘l2’, ‘solver’: ‘newton-cg’} |
| VAE | RandomForest | {‘bootstrap’: False, ‘max_depth’: 10, ‘max_features’: |
| | | ‘auto’, ‘min_samples_leaf’: 1, ‘min_samples_split’: 2, ‘n_ |
| | | estimators’: 100} |
| VAE | SVC | {‘C’: 1000, ‘gamma’: 0.1, ‘kernel’: ‘rbf’} |

TABLE 17c
Test Performance Encoder Latent output as input to supervised models In order to
minimize false alarms, most importance is assigned to precision indicated by a high F0.5 score.

| AutoEncoder | Classifier | TN | FP | FN | TP | precision | recall | F0.5 | F1 |
|---|---|---|---|---|---|---|---|---|---|
| AE | AdaBoost | 261 | 13 | 36 | 10 | 43.48 | 21.74 | 36.23 | 28.99 |
| AE | Gradient Boosting | 270 | 4 | 39 | 7 | 63.64 | 15.22 | 38.89 | 24.56 |
| AE | Logistic Regression | 272 | 2 | 42 | 4 | 66.67 | 8.70 | 28.57 | 15.38 |
| AB | Random Forest | 264 | 10 | 39 | 7 | 41.18 | 15.22 | 30.70 | 22.22 |
| AE | SVC | 274 | 0 | 45 | 1 | 100.00 | 2.17 | 10.00 | 4.26 |
| ATT | AdaBoost | 255 | 19 | 32 | 14 | 42.42 | 30.43 | 39.33 | 35.44 |
| ATT | Gradient Boosting | 268 | 6 | 41 | 5 | 45.45 | 10.87 | 27.78 | 17.54 |
| ATT | Logistic | 272 | 2 | 39 | 7 | 77.78 | 15.22 | 42.68 | 25.45 |
| | Regression | | | | | | | | |
| ATT | Random Forest | 264 | 10 | 36 | 10 | 50.00 | 21.74 | 39.68 | 30.30 |
| ATT | SVC | 273 | 1 | 43 | 3 | 75.00 | 6.52 | 24.19 | 12.00 |
| VAE | AdaBoost | 249 | 25 | 44 | 2 | 7.41 | 4.35 | 16.49 | 5.48 |
| VAE | Gradient Boosting | 272 | 2 | 46 | 0 | 0.00 | 0.00 | 0.00 | 0.00 |
| VAE | Logistic Regression | 266 | 8 | 46 | 0 | 0.00 | 0.00 | 0.00 | 0.00 |
| VAE | Random Forest | 273 | 1 | 46 | 0 | 0.00 | 0.00 | 0.00 | 0.00 |
| VAE | SVC | 267 | 7 | 44 | 2 | 22.22 | 4.35 | 12.20 | 7.27 |

| | | | | | | | | |
|---|---|---|---|---|---|---|---|---|
| Benchmark: LSTM Classifier | 271 | 4 | 12 | 1 | 20.00 | 7.69 | 15.15 | 11.11 |

The final performance of the hybrid models using the solely the encoder output of the autoencoder on the test set is shown in Table 17c. For the normal autoencoders, the hybrid combination with gradient boosting has best performance, with a precision of 63.64% and recall 15.22%. The attention autoencoder combined with logistic regression obtains highest performance for the deep hybrid models which only utilize the encoder output. All variational autoencoder hybrid models seem to drastically over-fit as the final test performance is extremely low. These deep hybrid models provide many false alarms shown by the many false positives, whereas they are only capable of detecting at most 2 anomalies. However, the table immediately shows only utilizing the latent space of the autoencoder scores worse than the performance of using the Mean Squared Error per minute.

### 7.4.3 Validation and Test Split Sensitivity Analysis

The cross-validation performance in the previous two sections showed for each model the standard deviation of all performance measures is relatively high. Indicating that within the validation set, which was used for training the supervised models, differences exists and generalization is likely to be difficult. This is probably again caused due to the very small validation set. Additionally, the hybrid models utilizing the latent vector are extremely over-fitting. Contrary, the deep hybrid models utilizing the reconstruction error per minute did achieve comparable test results as the cross-validation performance. However, the Section 7.3.2 already demonstrated the performance of the threshold detection models was also affected by the chosen validation and test split. Therefore, this section explores the effects of using different validation and test splits using the obtained hyperparameters.

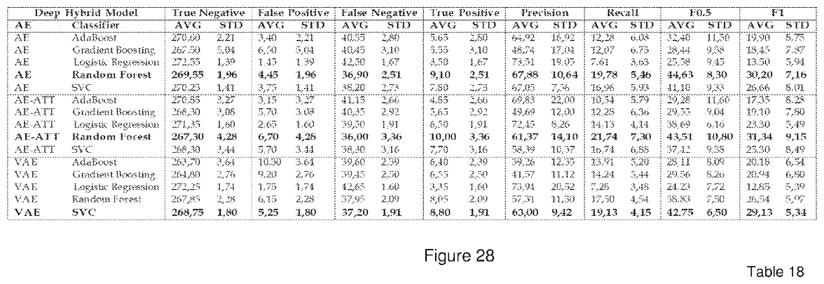

FIG. 28 lists the average test performance over 20 different train test splits for deep hybrid models using the reconstruction error vectors as input. Similar average hybrid performance results are obtained compared to the results of the single split in Section 7.4.1. In the table, it can be observed on average the highest Precision and F0.5 performance is obtained for the deep hybrid model consisting of the normal Autonencoder combined with a Random Forest model. This hybrid model configurations detects on average 9 anomalies accompanied with 4.5 false positives and is considered as the best deep hybrid model. However, the hybrid models consisting of attention autoencoder and random forest and VAE with SVC only have a slightly lower average F0.5 performances. Thus likewise to Section 7.3.2, but based on the average split results, the best hybrid models combine normal AE with RF, attention AE with RF and VAE with SVC. For each autoencoder type, these hybrid combinations are capable of detecting on average most true positives. It must be mentioned and similar to Section 7.3.2, for all deep hybrid model configurations a relatively high standard deviation can be observed which implies that generalization is likely to be difficult.

Further, if we compare the supervised learning models in FIG. 28, it may be observed that each deep hybrid combination with logistic regression is capable of achieving on average the highest precision. As explained in Chapter 6, logistic regression is one of the simplest machine learning algorithms and it can apparently in this case be used to obtain a high precision. However, this large precision is accompanied with large standard deviation and low recall values. This low generalization capability is probably caused due to the many input features and multi-collinearity generated by the autoencoders. As explained in Chapter 6, logistic regression is known to be affected by multi-collinearity or outliers present in the data. It seems a logical assumption the error vectors over time are related with the previous error vector which induces the collinearity. As expected, the random forest seems to have a better performance. As explained in Chapter 6, due to its nature random forest are (relatively to other well known methods) not prone to over-fitting, have good tolerance against noise or outliers and are not sensitive to multicollinearity. If we compare the two boosting algorithms, it seems having stump decision trees as used in Adaboost achieves better performance compared to the gradient boosting. On the other hand, SVMs are designed to minimize structural risk which describes the over-fitting and probability of misrepresenting unseen data. Table 18 shows for all autoencoder types the SVM also perform reasonable.

### 7.4.3.1 Latent Output of Encoder Sensitivity Analysis

Sections 7.4.1 and 7.4.2 already showed the performance of the latent encoder output, for the standard train, validation and test split, is worse compared to the hybrid models using the reconstruction error vector. Although the performance of models only utilizing the output of the encoder on a single split is lower, the effect of the chosen splits is still examined. Table 18 lists the average test performance over 20 different train test splits for deep hybrid models using the latent vectors as input. As expected the average performance results, shown in Table 18, also achieve worse performance. For this input type, both the normal AE combined with AdaBoost and the attention autoencoder combined with logistic regression are capable of obtaining the highest average F0.5. The normal autoencoder model is capable of detecting on average of 11 anomalies, but has a low average precision of 45.24%, recall of 23.91% and an F0.5 of 37.93%. For the attention autoencoder best performance is obtained by combining the autoencoder with a logistic regression classifier, resulting in an average precision of 72.85%, but it has a low average recall of 13.8%. Section 7.4.2 already showed bad performance of hybrid models which utilize the latent vectors from the VAE as input on a single validation and test split. As expected, also poor test average performance is obtained for the latent vector of the VAE autoencoder. All deep hybrid configurations using the VAE encoder have a low average precision below 20 percent and a recall below 10 percent. When comparing both supervised input types, it is observed the reconstruction error per minute vectors have better performance than the latent vectors. Therefore, for further explorations the deep hybrid models using the reconstruction error per minute as input vector are used.

TABLE 40
Latent Vector-Te-t Performance-Different Validation Test Splits

| Deep Hybrid | | True | | False | | False | | Positive | | | | | | | | | |
|---|---|---|---|---|---|---|---|---|---|---|---|---|---|---|---|---|---|
| Model | | Negative | | Positive | | Negatie | | True | | Precision | | Recall | | F0.5 | | F1 | |
| AE | Classifier | AVG | STD | AVG | STD | AVG | STD | AVG | STD | AVG | STD | AVG | STD | AVG | STD | AVG | STD |
| AE | AdaBoost | 260.20 | 4.94 | 13.80 | 4.94 | 35.00 | 2.51 | 11.00 | 2.51 | 45.24 | 8.53 | 23.91 | 5.46 | 37.93 | 6.53 | 30.89 | 5.70 |
| AE | Gradient | 263.65 | 3.92 | 10.35 | 3.92 | 37.80 | 3.05 | 8.20 | 3.05 | 44.86 | 10.77 | 17.83 | 6.64 | 33.57 | 8.14 | 24.98 | 7.46 |
| | Boosting | | | | | | | | | | | | | | | | |
| AE | Logistic | 270.50 | 2.69 | 3.50 | 2.69 | 41.15 | 2.30 | 4.85 | 2.30 | 61.09 | 14.84 | 10.54 | 5.00 | 29.06 | 10.41 | 17.38 | 7.40 |
| | Regression | | | | | | | | | | | | | | | | |
| AE | Random | 266.60 | 3.60 | 7.40 | 3.60 | 38.30 | 2.70 | 7.70 | 2.70 | 53.04 | 14.53 | 16.74 | 5.86 | 35.59 | 8.48 | 24.79 | 7.26 |
| | Forest | | | | | | | | | | | | | | | | |
| AE | SVC | 273.40 | 1.35 | 0.60 | 1.35 | 43.70 | 0.80 | 2.30 | 0.80 | 87.33 | 20.01 | 50.0 | 1.74 | 19.69 | 5.74 | 9.35 | 3.07 |
| AE- | AdaBoost | 256.70 | 5.27 | 17.30 | 5.27 | 34.75 | 3.35 | 11.25 | 3.35 | 39.55 | 9.50 | 24.46 | 7.29 | 34.82 | 8.49 | 29.86 | 7.84 |
| ATT | | | | | | | | | | | | | | | | | |
| AE- | Gradient | 264.00 | 3.16 | 10.00 | 3.16 | 38.75 | 2.15 | 7.25 | 2.15 | 42.58 | 12.41 | 15.76 | 4.67 | 31.38 | 8.29 | 22.78 | 6.22 |
| ATT | Boosting | | | | | | | | | | | | | | | | |
| AE- | Logistic | 271.40 | 2.11 | 2.60 | 2.11 | 39.65 | 2.39 | 6.35 | 2.39 | 72.85 | 15.78 | 13.80 | 5.20 | 37.95 | 9.88 | 22.77 | 7.33 |
| ATT | Regressinon | | | | | | | | | | | | | | | | |
| AE- | Random | 264.50 | 3.87 | 9.50 | 3.87 | 38.60 | 2.64 | 7.40 | 2.64 | 44.13 | 14.42 | 16.09 | 5.75 | 32.10 | 10.10 | 23.22 | 7.66 |
| ATT | Forest | | | | | | | | | | | | | | | | |
| AE- | SVC | 271.20 | 2.24 | 2.80 | 2.24 | 40.85 | 1.81 | 5.15 | 1.81 | 68.66 | 20.11 | 11.20 | 3.94 | 32.61 | 9.13 | 18.90 | 6.05 |
| ATT | | | | | | | | | | | | | | | | | |
| VAE | AdaBoost | 253.90 | 5.72 | 20.10 | 5.72 | 42.15 | 1.81 | 3.85 | 1.81 | 15.91 | 6.23 | 8.37 | 3.94 | 13.23 | 5.38 | 10.78 | 4.67 |
| VAE | Gradient | 269.65 | 2.89 | 4.35 | 2.89 | 45.20 | 1.01 | 0.80 | 1.01 | 15.10 | 17.99 | 1.74 | 2.19 | 5.64 | 6.81 | 3.05 | 3.78 |
| | Boosting | | | | | | | | | | | | | | | | |
| VAE | Logistic | 272.40 | 1.88 | 1.60 | 1.88 | 45.70 | 0.66 | 0.30 | 0.66 | 9.35 | 23.85 | 0.65 | 1.43 | 2.29 | 4.85 | 1.17 | 2.54 |
| | Regression | | | | | | | | | | | | | | | | |
| VAE | Random | 273.05 | 1.57 | 0.95 | 1.57 | 45.85 | 0.37 | 0.15 | 0.37 | 11.67 | 31.11 | 0.33 | 0.80 | 1.43 | 3.50 | 0.63 | 1.54 |
| | Forest | | | | | | | | | | | | | | | | |
| VAE | SVC | 264.70 | 4.46 | 9.30 | 4.46 | 44.00 | 0.92 | 2.00 | 0.92 | 19.23 | 9.55 | 4.35 | 1.99 | 10.93 | 4.60 | 6.91 | 2.98 |

### 7.5 Comparison Threshold Method Against Deep Hybrid Models

As explained above, the deep hybrid models utilizing the mean squared error per minute output of the autoencoder have higher performance compared to the latent vector ones. Therefore, for further analysis only these deep hybrid models are used. On average, the best threshold performance is obtained for the normal autoencoder with the F0.5 threshold, whereas the best deep hybrid performance is obtained combining the normal autoencoder with the random forest classifier. Table 19 shows the average and standard deviation of the true negatives, false positives, false negatives, true positives, precision and recall for both models. For both models, 20 different validation test splits are used. In the table, it can be observed both models have similar performance. The Deep hybrid has a slightly higher average precision and slightly lower standard deviation, where its recall is slightly lower with higher standard deviation. One advantage of the deep hybrid model over the standard autoencoder threshold detection method, is that the deep hybrid model facilitates the use of shapley values to interpret the model, which is further explained in Section 8.1.

TABLE 19
Comparison best models It is desired to have both precision and recall as high
as possible, but in order to minimize false alarms most importance is assigned to precision

| | True Negative | | False Positive | | False Negative | | True Positive | | Precision | | Recall | |
|---|---|---|---|---|---|---|---|---|---|---|---|---|
| Autoencoder | AVG | STD | AVG | STD | AVG | STD | AVG | STD | AVG | STD | AVG | STD |
| AE Random | 269.55 | 1.96 | 4.45 | 1.96 | 36.90 | 2.51 | 9.10 | 2.51 | 67.88 | 10.64 | 19.78 | 5.46 |
| Forest | | | | | | | | | | | | |
| AE F0.5 | 268.1 | 5.07 | 5.85 | 5.07 | 35.45 | 2.25 | 10.55 | 2.25 | 67.78 | 11.39 | 22.93 | 4.88 |
| Threshold | | | | | | | | | | | | |

### 7.5.1 Inspection of Misclassifications

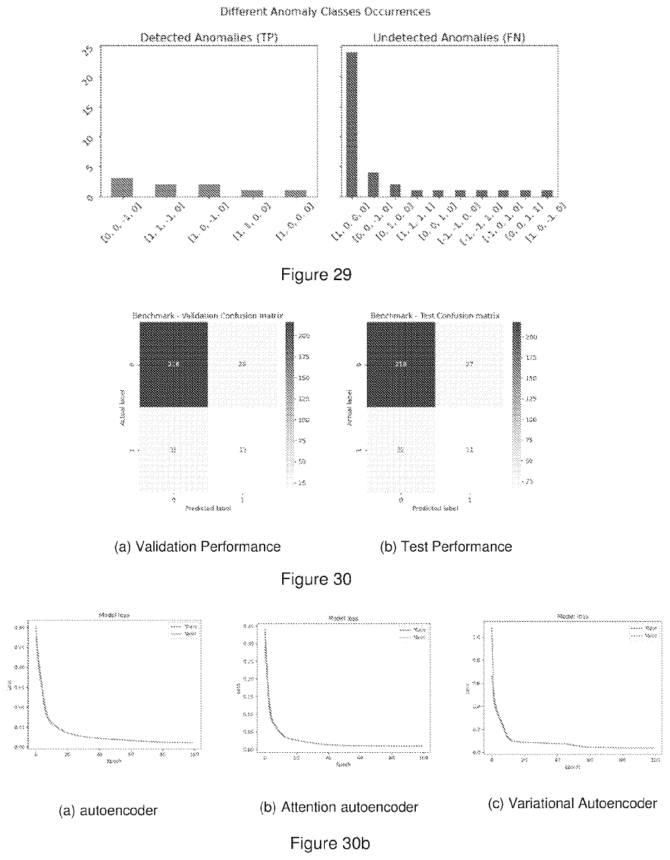

None of the deep autoencoder anomaly detection models seem to be capable to obtain a good performance with both high precision and recall. Therefore, this section inspects the miss classifications based on the labels and on the final chocolate properties. Although the actual values of the properties were not used during modeling, it is still possible to inspect them. In Chapter 5 we have seen that sequences with only a too high viscosity are the major anomaly type. FIG. 29 shows the distribution of the labels of anomalies categorized upon the detected and undetected anomalies. It can be observed the model has a hard time in detecting this majority anomaly class. As a result it is chosen to further inspect the specification thresholds by categorizing the test set based on its prediction outcome and inspect the property values.

A large part of the false negatives are mainly from the majority anomaly class, and are centred near the specification limit. At the same time, a large group of true negative sequences exist which also have a viscosity value closely to the specification limit. Analysis shows the other three properties of the majority anomaly class which remains undetected is almost always between control limits. For the yield and fat content property, it can be observed although the value is far above the upper specification limit still the anomaly remains undetected. These observations have been discussed with several quality technicians within Mars and this might be the result of poorly defined specification limits. Moreover, the quality operators mention the manual influence an experienced operator can perform before the chocolate sample is smeared on the inspection plate. Therefore, it is also checked whether using the control limits instead of specification limits improves the model.

### 7.6 Control Limits as Labels

As there is doubt about the quality of the labels, the influence of using different labels is evaluated. Moreover, during the data exploration phase, box plots were used to graphically inspect the chocolate properties on their quartiles. The box plots showed the actual control limit values of all four properties were in line with the interquartile range or the actual whiskers of the box plot distributions. The whiskers in the box plot indicate the minimum and maximum of the quartile range and points outside this range are considered as anomalies. This observation strengthens the doubts into the quality of the labels. Therefore, similar as for the specification limits labels the data is again split in a train, validation and test set. The train set consists of exclusively 70 percent of the normal samples. The train set is first used to fit a new min max scaler. In order to prevent data leakage, all sequences are scaled using the fitted scaler and after training the autoencoders the training set is discarded. The remainder of this section first explains the benchmark model in Section 7.6.1, which will be used to compare the semi-supervised anomaly detection approach against a supervised classification model. Further, Section 7.6.2 explains the optimization of the different autoencoders and visualizes the attention weight plots generated by the attention-based LSTM autoencoder. The anomaly detection results by setting a threshold are shown in Section 7.6.3 and the deep hybrid anomaly detection results are shown in Section 7.6.4.

TABLE 20

| Control Limit as Binary Labels Sample set sizes | | | | |
|---|---|---|---|---|
| Type | Train | Validation | Test | Total |
| Normal | 1138 | 244 | 245 | 1627 |
| Anomaly | — | 145 | 145 | 290 |

### 7.6.1 Benchmark Model Supervised Classification

Again a supervised binary benchmark model is developed which is used to compare the performance of semi-supervised anomaly detection models. For this supervised classification model, same hyperparameters and training method as described in Section 7.1 are used. The samples are again split into 70% training, 15% validation and 15% test set in a stratified manner. The classifier composed of two hidden layers, with respectively 16 neurons and 8 neurons, trained using a learning rate of 0.0001 and a batch size of 16 obtained the best validation performance. The validation and test confusion matrices are shown in FIG. 30. Compared to the benchmark model for the specification limit anomalies, the test performance of the benchmark model for this label type improved. The test performance obtains a precision of 28.9%, recall of 25.6%, F0.5 of 27.1% and an F1 of 28.2%. Although the performance is still quite low, it is expected that the performance of the detection methods is better.

### 7.6.2 Model Architecture and Parameters

For this data set the three autoencoder types are trained using the same hyperparameters as for the data set which uses the specification limits as labels. FIG. 30b illustrates the training and validation loss during training for the best performing autoencoder, attention autoencoder and variational autoencoder. During training the autoencoders all three models approach a loss of zero, indicating that the three types of autoencoders learn a representative representation of the right chocolate batches process data. Trained autoencoders which learned the “in control” behaviour are further used to detect anomalies, which is shown in Sections 7.6.3 and 7.6.4.

### 7.6.2.1 Attention Weight Plots

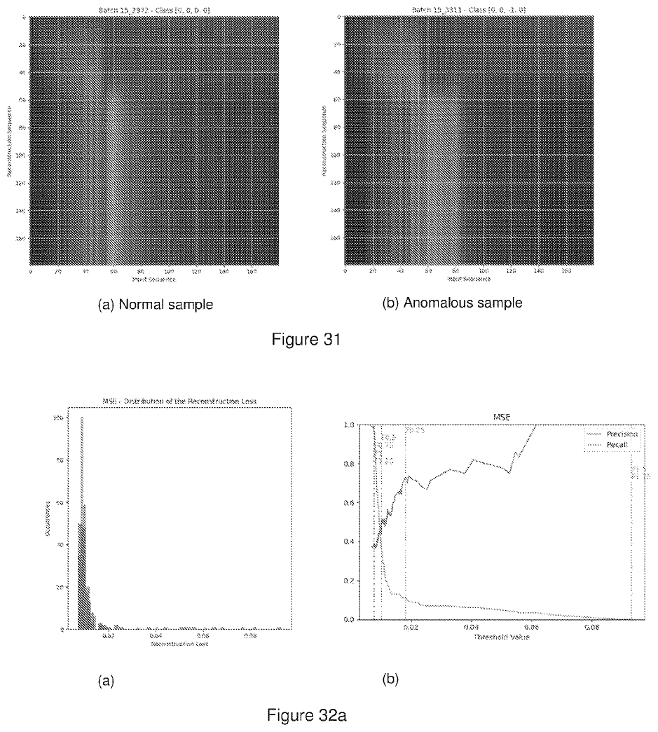

FIG. 31 shows the attention weight plots for the reconstruction of a normal and anomalous sample. The figure shows which input minutes are important for the reconstructing the sequence. The attention weight plots of both samples look quite similar. In both plots, we observe that the first twenty minutes receive little attention. After the first twenty minutes and until 60 minutes, the attention moves directly proportional. Further, we observe a straight vertical line of attention weights starting at input 60. The vertical line indicates the input around one hour seems to gain consistent of attention for the remainder of the reconstruction. However, differences exist as for the anomalous sample the period which gains consistent attention is longer and focuses from 60 till 80 minutes. In case of the normal sample this surface centers near 55 and 65 minutes. It is likely that filling the conche for the production of the anomalous sample took longer, which is remembered during the reconstruction of the sequence by assigning more weights to it. This figure again shows the autoencoder with attention mechanism can build context-aware representations.

### 7.6.3 Anomaly Detection by Setting a Threshold

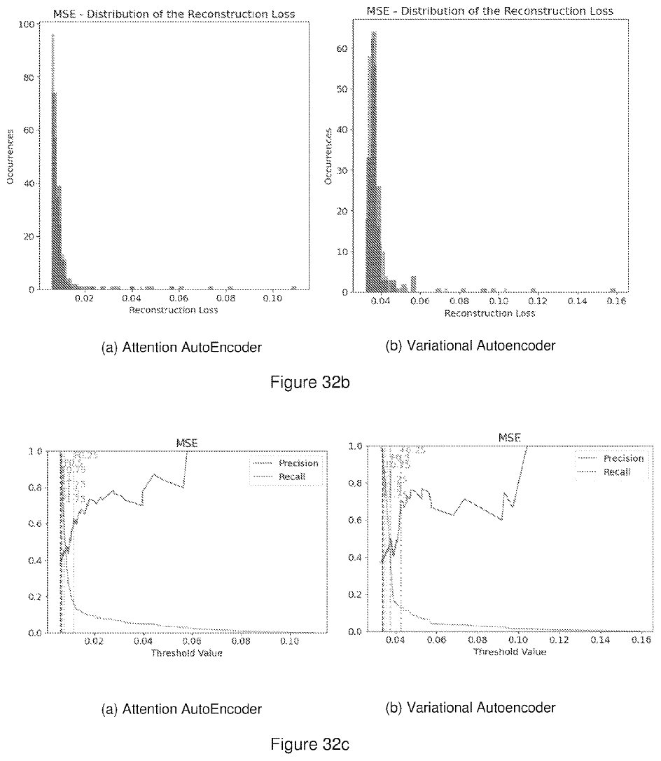

If we explore the MSE reconstruction loss distributions categorized by the labels for the normal autoencoder in FIG. 32a, it can be seen that a large part of the distributions overlap. The distributions for the attention autoencoder and variational autoencoder in FIG. 32b look similar. Earlier in Section 7.3.1, it was shown overlapping mean squared error distributions make it difficult to set a threshold which is capable of achieving both high recall and precision. FIG. 32a(b) shows the optimal threshold values for different FB values for the normal autoencoder. Using the validation set only the F0.25 threshold seems to obtain a reasonable performance, where it has high precision, but unfortunately again a low recall. Again, similar observations can be made for the threshold curves of the attention and variational autoencoder, shown in FIGS. 32c(a) and 32c(b). As a result, for all three autoencoder types using the validation set the optimal threshold can be determined using the F0.25 score, which obtains a relatively high precision but low recall.

The F0.25 threshold and its validation and test performance are shown in Table 21. Here it can be observed that the normal autoencoder with MSE F0.25 threshold achieves good test performance. The normal autoencoder achieves a high 90 percent precision on the test set, but has low recall of about 15 percent. The attention autoencoder has a lower precision of 65 percent, but detects more anomalies and thus has a higher recall of almost 20 percent. Finally, the variational autoencoder has a precision of about 80 percent and a recall of 15 percent. It can be observed the normal autoencoder has obtained best performance when the three different autoencoders are compared. Comparing the autoencoder threshold detection methods against the supervised benchmark model, it can be observed that the autoencoders achieve higher precision and thus higher F0.5 scores. Although, the benchmark LSTM classifier is capable of obtaining a higher recall, the result again validates the choice for the semi-supervised anomaly detection approach. However, for all three autoencoder types, it can be observed the test performance is higher compared to the validation performance. Therefore, there is a chance that the used test set consists of a better representation of the training data compared to the validation. Indicating that the anomalies in the test set are more distinctive from the normal learned behaviour compared to the anomalies in the validation set.

TABLE 21
Overview autoencoders Threshold Performance. In order to minimize false
alarms, most importance is assigned to precision indicated by a high F0.5 score

| | F0.25 | Validation | | | | | | | Test | | | | | | |
|---|---|---|---|---|---|---|---|---|---|---|---|---|---|---|---|
| Model | Threshold | TN | FP | FN | TP | Precision | Recall | F0.5 | TN | FP | FN | TP | Precision | Recall | F0.5 |
| Autoencoder | 0.0181 | 238 | 6 | 129 | 16 | 72.73 | 11.03 | 34.33 | 243 | 2 | 124 | 21 | 91.30 | 14.48 | 44.30 |
| Attention | 0.0115 | 230 | 14 | 121 | 24 | 63.16 | 16.55 | 40.40 | 230 | 15 | 117 | 28 | 65.12 | 19.31 | 44.16 |
| VAE | 0.0425 | 236 | 8 | 126 | 19 | 70.37 | 13.10 | 37.55 | 239 | 6 | 123 | 22 | 78.57 | 15.17 | 42.80 |

| | | | | | | | | | | | | | | |
|---|---|---|---|---|---|---|---|---|---|---|---|---|---|---|
| Benchmark | 216 | 28 | 31 | 13 | 31.71 | 29.55 | 31.25 | 218 | 27 | 32 | 11 | 28.95 | 25.58 | 28.21 |

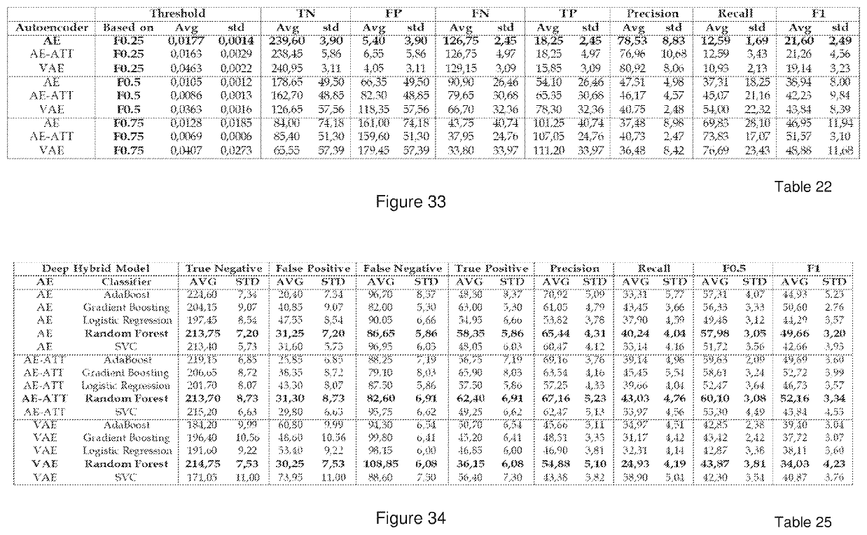

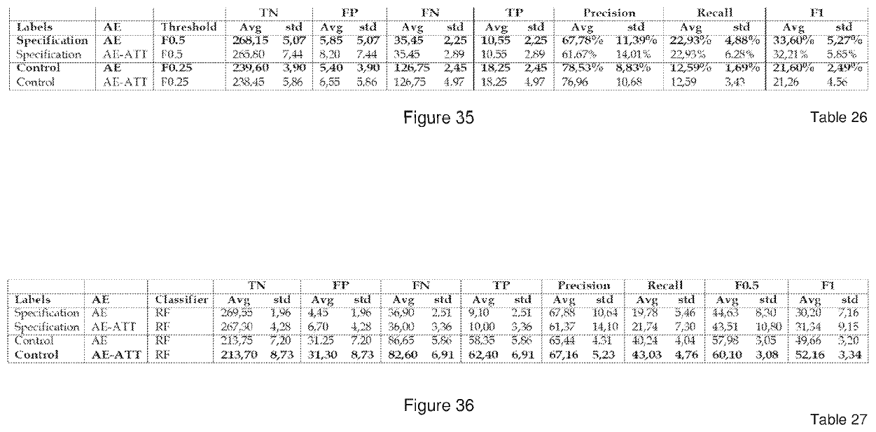

As a result and similar to Section 7.3.2, again the effect of having different validation and test split is explored. FIG. 33 shows the average test performance over 20 different train test splits. For each of these split the thresholds are determined based upon the highest F0.25, F0.5 and F0.75 value. Unlike the results of the specification limits in Section 7.3.2, for the control limit labels the F0.25 threshold obtains best trade off in terms of high precision and recall. The overall best trade off is obtained for the normal autoencoder anomaly detection model. This autoencoder on average achieves a precision of about 79 percent and a recall of only about 13 percent, with a relatively high standard deviation of respectively 9 and 2 percent. The autoencoder obtains a slightly lower average precision and recall of 77 and 13 percent with a higher standard deviation of respectively 11 and 3 percent. The variational autoencoder obtains highest average precision equalling 81 percent with a standard deviation of 8, but its recall performance is worse because the mean and standard deviation equal 11 and 2 percent. As a result the trade off between precision score and recall is best for the normal autoencoders. Due to the overlapping MSE distributions for all autoencoder types the other two thresholds can only obtain a precision of at most 50 percent but accompanied with an equally high recall. These precision and recall result in a relatively high F1 performance score, compared to the other lower threshold. However, setting such a threshold for a detection model would yield many false alarms and is undesired. Moreover, when looking at the standard deviations we can observe relatively high standard deviations for all models.

### 7.6.4 Deep Hybrid Anomaly Detection

Similar to Section 7.4.1, the performance of the deep hybrid models for the control labeled anomalies is also examined. Fully trained autoencoders are used to reconstruct the 180 length sequences and then for each minute the mean squared error is calculated. This error vector serves as input for the deep hybrid anomaly detection methods. Again, the hyper hyperparameters for the supervised learning classifiers are obtained using K-fold cross-validation using the validation set. The cross-validation results for the best performing hybrid models are shown in Table 23a and the actual hyperparameters are shown in Table 23b. The results show the precision and recall of the hybrid models consisting of the autoencoder and attention autoencoder are all similar. The precision and recall centers respectively close to 60 and 40 percent. However, the obtained standard divisions are relatively high compared to the obtained precision and recall scores, indicating that generalization can be difficult due to over-fitting. For both autoencoder types the random forest seems to be the best performing combination. Similar to the specification labels, the hybrid models using the output of the VAE perform worse. Based on the cross-validation results this model can best be combined with the gradient boosting algorithm.

TABLE 23a
Cross-validation Hyperparameter Tuning Results Reconstruction error per
minute as input to supervised models In order to minimize false alarms, most importance is assigned
to precision indicated by a high F0.5 score.

| | | Precision | | Recall | | F0.5 | | F1 | |
|---|---|---|---|---|---|---|---|---|---|
| Type | Classifier | Avg | Std | Avg | Std | Avg | Std | Avg | Std |
| AE | AdaBoost | 63.27 | 11.16 | 41.79 | 11.55 | 56.84 | 10.20 | 49.78 | 10.58 |
| AE | Gradient Boosting | 60.28 | 7.14 | 47.64 | 8.47 | 56.99 | 6.86 | 52.89 | 7.13 |
| AE | Logistic Regression | 56.19 | 4.92 | 45.88 | 8.79 | 53.54 | 5.44 | 50.22 | 6.74 |
| AE | Random Forest | 67.20 | 13.05 | 43.49 | 7.31 | 60.14 | 9.71 | 52.36 | 7.82 |
| AE | SVC | 58.64 | 10.36 | 40.35 | 10.28 | 53.66 | 10.50 | 47.69 | 10.53 |
| ATT | AdaBoost | 62.89 | 10.79 | 37.97 | 6.32 | 55.29 | 8.54 | 47.05 | 7.01 |
| ATT | Gradient Boosting | 62.77 | 11.29 | 43.49 | 9.87 | 57.51 | 11.01 | 51.23 | 10.58 |
| ATT | Logistic Regression | 53.88 | 5.56 | 38.66 | 8.02 | 49.73 | 6.05 | 44.77 | 6.95 |
| ATT | Random Forest | 64.61 | 10.00 | 44.53 | 7.67 | 58.89 | 8.32 | 52.32 | 7.55 |
| ATT | SVC | 58.98 | 3.98 | 37.29 | 6.99 | 52.64 | 5.19 | 45.50 | 6.30 |
| VAE | AdaBoost | 47.45 | 7.70 | 37.64 | 10.72 | 144.44 | 6.57 | 41.15 | 7.48 |
| VAE | Gradient Boosting | 48.24 | 7.80 | 37.32 | 10.66 | 45.15 | 8.08 | 41.55 | 8.94 |
| VAE | Logistic Regression | 44.41 | 4.89 | 34.51 | 5.35 | 41.93 | 4.86 | 38.74 | 4.99 |
| VAE | Random Forest | 55.84 | 10.87 | 27.67 | 11.30 | 44.57 | 9.80 | 35.61 | 10.76 |
| VAE | SVC | 42.47 | 3.16 | 42.77 | 1.83 | 42.45 | 2.25 | 42.50 | 1.06 |

TABLE 23b
Control-Limits Labels-HyperParameter Cross Validation Optimal Parameters

| AE | Classifier | Params |
|---|---|---|
| AE | AdaBoost | {‘learning_rate’: 0.01, ‘n_estimators’: 100} |
| AE | GradientBoosting | {‘learning_rate’: 0.1, ‘max_depth’: 10, |
| | | ‘min_samples_leaf’: 4, |
| | | ‘min_samples_split’: 10, ‘n_estimators’: 200} |
| AE | LogisticRegression | {‘C’: 1000, ‘penalty’: ‘l2’, ‘solver’: ‘liblinear’} |
| AE | Random Forest | {‘bootstrap’: True, ‘max_depth’: 50, ‘max_features’: |
| | | ‘auto’, ‘min_samples_leaf’: 3, ‘min_samples_split’: 2, ‘n_ |
| | | estimators’: 100} |
| AE | SVC | {‘C’: 10, ‘gamma’: 10, ‘kernel’: ‘rbf’} |
| ATT | AdaBoost | {‘learning_rate’: 0.01, ‘n_estimators’: 500} |
| ATT | GradientBoosting | {‘learning_rate’: 0.1, ‘max_depth’: 10, |
| | | ‘min_samples_leaf’: 4, |
| | | ‘min_samples_split’: 2, ‘n_estimators’: 500} |
| ATT | LogisticRegression | {‘C’: 1000, ‘penalty’: ‘l2’, ‘solver’: ‘lbfgs’} |
| ATT | RandomForest | {‘bootstrap’: True, ‘max_depth’: 10, ‘max_features’: |
| | | ‘auto’, ‘min_samples_leaf’: 3, ‘min_samples_split’: 10, ‘n_ |
| | | estimators’: 1000} |
| ATT | SVC | {‘C’: 100, ‘gamma’: 1, ‘kernel’: ‘rbf’} |
| VAE | AdaBoost | {‘learning_rate’: 0.1, ‘n_estimators’: 1000} |
| VAE | GradientBoosting | {‘learning_rate’: 0.1, ‘max_depth’: None, ‘min_samples_leaf’: |
| | | 3, ‘min_samples_split’: 7, ‘n_estimators’: 200} |
| VAE | LogisticRegression | {‘C’: 1000, ‘penalty’: ‘l2’, ‘solver’: ‘liblinear’} |
| VAE | RandomForest | {‘bootstrap’: True, ‘max_depth’: 50, ‘max_features’: |
| | | ‘auto’, ‘min_samples_leaf’: 1, ‘min_samples_split’: 7, ‘n_ |
| | | estimators’: 500} |
| VAE | SVC | {‘C’: 100, ‘gamma’: ‘scale’, ‘kernel’: ‘rbf’} |

### 7.6.4.1 Test Performance of Chosen Hyperparameters

The cross-validation results in Table 23, showed the obtained standard divisions are relatively high compared to the obtained precision and recall scores, indicating that generalization can be difficult due to over-fitting. However, if we explore the final test performance results in Table 24 it is observed the test performance is quite similar to the cross-validation performance. For this validation test split, the autoencoder seems to be best combined with the logistic regression, obtaining a precision, recall and F0.5 score of respectively 65, 39 and 59 percent. Almost similar results are obtained for the Attention autoencoder. The deep hybrid combinations with the normal autoencoder perform slightly better than the combinations with the attention autoencoder. However, the differences are quite small. This autoencoder type can best be combined with the Adaboost classifier, obtaining a precision, recall and F0.5 score of respectively 65, 41 and 58 percent. Again, the VAEs perform worse as it obtains its highest performance scores when the autoencoder is combined with adaboost with a precision, recall and F0.5 score of respectively only 57.14, 30.34 and 48.57 percent. Comparing all deep hybrid anomaly detection methods with the benchmark, it can be observed the benchmark model is outperformed by all other hybrid models.

TABLE 24
Test Performance Reconstruction error per minute as input to supervised
models In order to minimize false alarms, most importance is assigned to precision indicated by
a high F0.5 score.

| autoencoderClassifier | | TN | FP | FN | TP | Precision | Recall | F0.5 | F1 |
|---|---|---|---|---|---|---|---|---|---|
| AE | AdaBoost | 216 | 29 | 89 | 56 | 65.88 | 38.62 | 57.73 | 48.70 |
| AE | Gradient Boosting | 203 | 42 | 88 | 57 | 57.58 | 39.31 | 52.68 | 46.72 |
| AE* | Logistic | 218 | 27 | 89 | 56 | 67.47 | 38.62 | 58.70 | 49.12 |
| | Regression* | | | | | | | | |
| AE | Random Forest | 206 | 39 | 86 | 59 | 60.20 | 40.69 | 54.93 | 48.56 |
| AE | SVC | 209 | 36 | 95 | 50 | 58.14 | 34.48 | 51.12 | 43.29 |
| ATT | AdaBoost | 212 | 33 | 85 | 60 | 64.52 | 41.38 | 58.03 | 50.42 |
| ATT | Gradient Boosting | 191 | 54 | 89 | 56 | 50.91 | 38.62 | 47.86 | 43.92 |
| ATT | Logistic Regression | 215 | 30 | 92 | 53 | 63.86 | 36.55 | 55.56 | 46.49 |
| ATT | Random Forest | 209 | 36 | 90 | 55 | 60.44 | 37.93 | 54.03 | 46.61 |
| ATT | SVC | 201 | 44 | 87 | 58 | 56.86 | 40.00 | 52.44 | 46.96 |
| VAE | AdaBoost | 212 | 33 | 101 | 44 | 57.14 | 30.34 | 48.57 | 39.64 |
| VAE | Gradient Boosting | 183 | 62 | 92 | 53 | 46.09 | 36.55 | 43.80 | 40.77 |
| VAE | Logistic Regression | 176 | 69 | 87 | 58 | 45.67 | 40.00 | 44.41 | 42.65 |
| VAE | Random Forest | 197 | 48 | 96 | 49 | 50.52 | 33.79 | 45.97 | 40.50 |
| VAE | SVC | 165 | 80 | 98 | 47 | 37.01 | 32.41 | 35.99 | 34-56 |
| Benchmark- | LSTM Classifier | 218 | 27 | 32 | 11 | 28.95 | 25.58 | 28.21 | 27.16 |

### 7.6.4.2 Validation and Test Split Sensitivity Analysis

Due to the small sample sets again a sensitivity analysis on the validation and test set is performed. Results of the sensitivity analysis are shown in FIG. 34. The results in this table show that the best performing hybrid model during validation is not as robust. Earlier parameter optimization through cross-validation returned the autoencoder with logistic regression as the best validation model. However as explained before, the logistic regression is sensitive to multi-collinearity and the reconstruction error output of the autoencoder can possess auto-correlation. Therefore, the normal autoencoder can better be combined with the random forest which obtains a robust F0.5 score. This hybrid model has a precision on average of 64.44 percent with 4.31 standard deviation, a recall of 40 percent and standard deviation of 4 and final F0.5 performance of 58 percent with a standard deviation of 3 percent. Similar findings can be made for the attention autoencoder. Combining the attention autoencoder with the random forest model seems to be more robust compared to the other supervised learning algorithms. On average the random forest achieves a precision of 67 percent with a s.d. of 5, a recall of 43 with a s.d. of 5 percent and the final F0.5 performance of 60 percent with a standard deviation of 3 percent. It can thus be observed that the deep hybrid models with bagging random forest have best capabilities to predict whether a chocolate batch is anomalous, which is similar to the results in Section 7.4. Consistent with the other results, the VAE hybrid model performance is worse than the other two autoencoder types. The highest performance for the VAE hybrid model is obtained by combining it with the random forest.

Furthermore, if we inspect the standard deviations of all hybrid models, we observe that the standard deviations are much smaller compared to the models trained for specification limits in Section 7.4.3. Indicating that training the models using control limits are much more robust compared to the deep hybrid models trained on specification limit anomalies.

### 7.7 Comparing Models Trained on Different Labels

FIG. 36 compares the results of the anomaly detection methods which set a threshold for the mean squared error. This table compares the performance of using specification limits as label against using the control limits. Unfortunately for first label type no good performance is obtained. The best model for the detecting out of specification limit anomalies consists of the normal autoencoder and setting a threshold using the F0.5 threshold. On average this model obtains a 68 percent precision with a relatively large standard deviation of 11 percent, the recall equals on mean 23 percent with s.d. 5 percent. In terms of precision, the models detecting out of control anomalies score better. The best performing model is also the normal autoencoder which obtains an average precision of 78 percent with s.d. of 9 percent. However, their detection rate is much lower as the recall only equals about 12 percent. Consequently, it can be stated anomaly detection methods to detect out of control or out of specification chocolate batches by setting a threshold have low detection rates as both recall values are low. However, setting thresholds is a method which is capable of achieving relatively high precision for the out of control chocolate batches. The results of the latter label type are quite robust, as the standard deviations are not as large.

Above, it is explained that for both label types, the threshold methods have low detection rates. Choosing which type of model is best depends on the trade-off between the precision and recall. The detection rate of the threshold method for specification limit anomalies is higher, whereas the precision for the control limit anomalies threshold method is much higher. Further, in Section 7.5 it was already stated that for the specification limit anomalies, the deep hybrid methods have similar performance as the threshold method. However, the deep hybrid models for the out of control limit anomalies seem to outperform all other models due to its higher detection rate. FIG. 36 compares the deep hybrid models for both labeling types. The table shows that the attention autoencoder combined with a Random Forest is capable of achieving on average a precision of 67 percent with a standard deviation of 5 percent, combined with a recall of 43 percent with a standard deviation of 5 percent. The performance of this model on average achieves an F0.5 score of 60 percent, with a moderate standard deviation of 3 percent.

### 7.8 Concluding Remarks

In this chapter, we have trained multiple unsupervised autoencoders to learn the normal process behaviour of chocolate batches. Results have validated the anomaly detection approach over the straightforward supervised classification approach. For practical reasons, the specification limits were used to classify each batch of chocolate. The learned normal behaviour could facilitate the detection of an incorrect chocolate batch. First, for each sequence length the hyperparameters of an LSTM autoencoder, LSTM autoencoder with multiplicative attention and an LSTM variational autoencoder were optimized such that lowest reconstruction loss was obtained. First insights were obtained by inspecting the attention weight plot for different samples, which all assigned more importance to the filling phase. Implying that the attention mechanism is able to learn a context aware representations. During the data exploration, it was observed the differences in patterns of single variables and first principal component over time between good and out of specification batches were quite small. This observation was confirmed by the reconstruction loss distributions plots. The mean squared error values of good and incorrect samples showed quite some overlap, which indicates the difficulty of the faced problem.

Anomaly detection can learn from good cases and provide an additional dimension to the data, but an important assumption accompanied with this method concerns that the distributions of the normal and anomaly data set are substantially different. As a result setting a threshold did not yield the desired performance. The highest average observed precision was only 68 percent and the recall was only 23 percent and both test performance measures showed large variance. Indicating that the anomaly threshold detection models are not robust. Moreover, it was observed the performance of the detection methods decreased as the length of the sequence increased. Indicating that the autoencoders which are trained exclusively on good behaviour learn more noise with longer sequences and as such shorter sequences are preferred. It was further investigated whether combining the output of unsupervised autoencoders with supervised learning models improved the prediction performance. For the autoencoder method, the majority of exclusive normal samples was used to learn the desired behaviour. Then only a small subset of both normal and anomalies was used to train the supervised method with the learned representations of the autoencoders. Two methods for such semi-supervised model were considered; one using the reconstruction error and the other using the output of the encoder as input vector to the supervised model. Results show the reconstruction error vector provided better differences between both data types, but the small subset available for training the supervised algorithms makes the deep hybrid model prone to over-fitting. As a result, the performance was still similar to the threshold performance. It is thus concluded, the autoencoder was not able to detect major differences between in within specification and outside specification chocolate batches, providing a noisy reconstruction error input for the supervised learning models.

As mentioned, for practical reasons the specification limits were used to define the labels of sequences. However, during the data exploration phase, box plots were used to graphically inspect the chocolate properties on their quartiles. The box plots showed the control limit values of all four properties also described either the interquartile range or the whiskers of the distributions. Discussing this observation together with the bad anomaly detection performance gave birth to doubts in the used specification labels. Therefore, also the effect of using the control labels was explored. The attention mechanism again highlighted the filling phase for the reconstruction of the sequence. Compared to the specification labels, setting a threshold for detecting out of control anomaly detection models is capable of obtaining higher precision but at the cost of an even lower recall. The variances of the test performance were also quite similar and thus relatively high. Leading to the conclusion that anomaly detection by setting a threshold on the reconstruction error is not sufficient for out of control chocolate batches. Contrary, the deep hybrid anomaly detection models showed more satisfying results. Evaluating the different supervised learning methods demonstrated the random forest as the dominating model to use within the deep hybrid model. Although the difference with the standard autoencoder was quite small, the autoencoder with attention yields the best performance. Compared to training the deep hybrid models with specification limits, the detection rate (recall) of out of control anomalies doubled, whereas the precision performance kept similar. Additionally the standard deviations of precision, F0.5 and F1 decreased by half, which yields a robust detection model. Indicating incorrect chocolate process behaviour can best be detected training autoencoders on chocolate production batches which are in control. Overall the best performing model detects out of control chocolate batches by combining the output of an Attention autoencoder with a Random Forest.

### 8 Explaining the Best Model

This chapter covers the evaluation phase of the CRISP-DM methodology by obtaining insights from the selected model from a business perspective. As explained in previous chapter, the best performing model combines the attention-based LSTM autoencoder with a random forest classifier to detect “out of control” anomalies. The results of the best model are examined and explained using SHAP values. Furthermore, it is explored how the model makes a certain classification in order to translate them to business insights.

### 8.1 SHAP Values of Best Performing Model

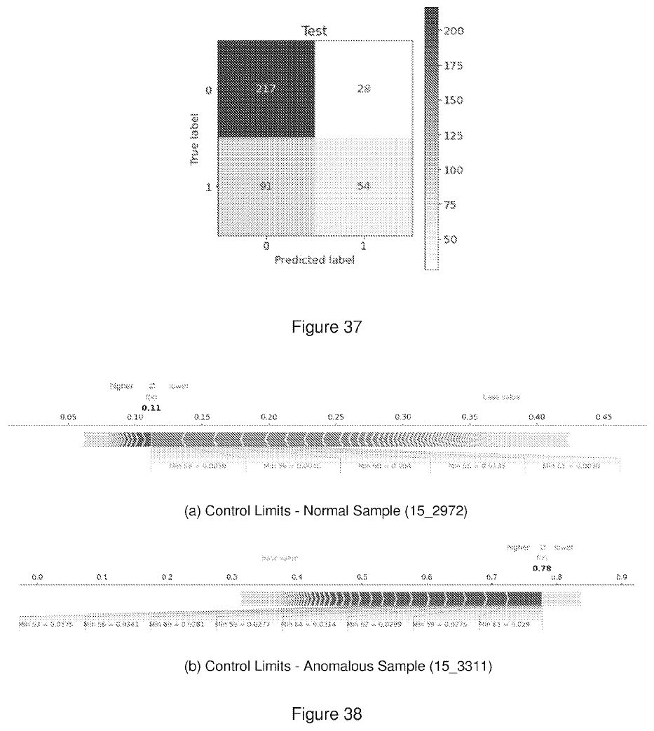

One major advantage of the hybrid anomaly detection method, which uses the reconstruction error per minute as input feature, is that interpretability is facilitated using SHaPley values. SHapley Additive explanations (SHAP) algorithm was first published by Lundberg and Lee (2017) and is a way to reverse-engineer the output of a machine learning algorithm. A single validation and test split is chosen to illustrate the interpretability using SHAP values. The confusion matrix is given in FIG. 37. The performance for the out of control anomaly detection model obtains a precision, recall, F0.5 and F1 of respectively 65.86, 37.24, 57.08 and 47.57 percent.

Local interpretability regards the analysis of individual samples which are predicted by the model. A force plot is used to show the SHAP values for both a normal and an anomalous sample and gives an idea of the contribution of features to an actual prediction. Before examining the results, it is important to note that these values do not serve as causal relations, but only provide insights to the associations between the process features and the target variable. As an example, FIG. 38 shows the SHAP values for two representative samples from the out of control test set. The prediction output of the force plot of the normal sample, as shown in FIG. 38a, equals 0.1. The figure plots the SHAP plots the top most influential features for the sample under study. Features in red color influence positively by dragging the prediction value closer to 1, whereas features in blue color do the opposite. The MSE value at minutes 56, 58, 59, 60 and 61 all contribute to the prediction of a normal sample. Furthermore, it can be observed the total surface of the red color is relatively small compared to the blue surface. Indicating that these minutes have much larger contributions to the final output of the model. The SHAP values for the anomalous sample are shown in FIG. 38b, the model predicts a value of 0.78. The contribution of the blue surface for this sample is vastly small because the stacked surface is only given in light blue. Implying that these features have low importance for the prediction output for this value. Moreover, it can be observed the MSE at minutes 53, 56, 60, 58, 64, 62, 59 and 61 drive the prediction of an anomaly. In both figures a base value close to 0.37 is given, which represents the average of all prediction outputs during training. In general, these individual SHAP value figures provide an intuitive insight in how the model makes a prediction. These examples can either be used to validate the results with domain knowledge, improve the domain knowledge or enhance the model by detecting potential noisy parts in time features.

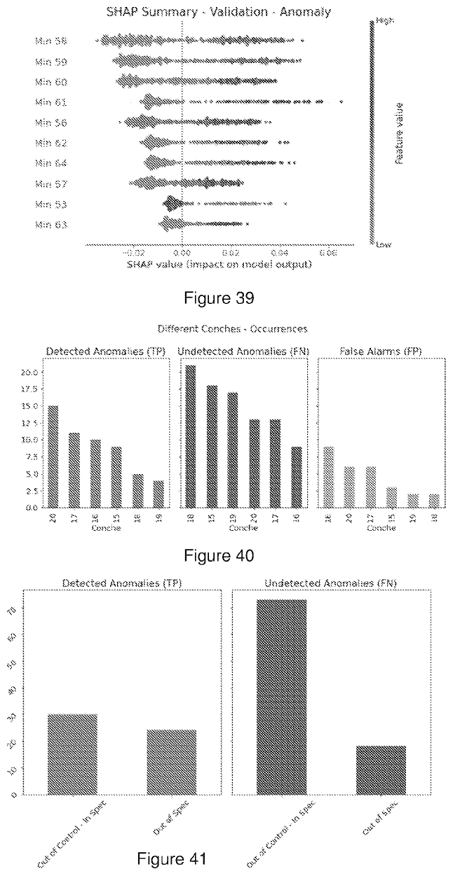

The collective SHAP values of the training set are used to examine the feature importance of the model, also known as the global interpretability. FIG. 39 shows a SHAP summary plot for the prediction of out of control anomalies. The summary plot lists the top contributing features of all samples used during training the random forest. The top ten features include the mean squared error at minutes 53, 56, 57, 58, 59, 60, 61, 62, 63, and

64. It can be observed the top ten most influential features all center around one hour. As explained in Section 7.2.1 and in Chapter 5, this is the time it takes until the conche is filled with all the required raw materials. Additionally, the value that each sample has at the specific feature is represented with the color. It is observed on all minutes, except minute 53, that a high error on these minutes typically pushes towards the prediction of the anomalous class. Combining the local SHAP values with the plot in FIG. 39 and the attention weight plots in Section 7.2.1, stresses a deviation from the standard filling process is more likely to be associated to incorrect chocolate properties. As such this research revealed the importance of the filling duration. Discussing this observation with quality technicians, reveals that a longer filling duration is either caused due to a manual pause in the system or due to a lower output by the air classifying mill.

### 8.2 Classification Inspections

FIG. 40 shows how the detection of the model is distributed across the machines. It may be observed that the model detects chronologically most incorrect batches produced for conches 16, 17 and 20. As expected, these batches have also the least undetected anomalies because the distribution of anomalies over all machines was quite even. However, simultaneously the detection algorithm also produces most false alarms for these machines. For conche 18 and 19, many anomalies remain undetected as the deep hybrid anomaly detection method detects only respectively 5 and 4 samples. The least amount of false alarms (3) is also given for these two machines. Although these results show, in terms of prediction modeling, there little differences between conches exist supporting the gut feeling of quality technicians. However, due to the very little occurrences this gut feeling cannot be tested and remains a gut feeling.

Moreover, on a high level anomalies can be divided into two different types by using the control limits as labels. An anomalous chocolate batch is either out of control but between specification limits or it is out of specification limits. The latter is the worst anomaly type because then further work is necessary. FIG. 41 shows that from all detected models, the model detects an about evenly amount of both types of samples. The figure shows the model detects about half of the out of specification limit samples, whereas only one third of the out of control but in specification samples become detected.

The Attention Weights plots in Sections 7.2.1 and 7.6 and the SHAP values in Section 8.1 stressed the importance of the filling phase for the final prediction of an anomaly. Therefore, it is chosen to inspect the fill duration values based on the final prediction outcome. Results show the median fill duration of the normal chocolate batches (true negatives) equals about 51 minutes, whereas the median of the false positives and true positives is higher and centers nearby 58 minutes. In terms of fill duration the group of undetected anomalies (false negatives) looks very similar to the group of normal batches. In case the fill duration is much higher compared to the normal behaviour, the anomaly detection model can identify an anomaly. However, the results also show the model is capable of detecting anomalies which have similar filling duration as the true negatives, indicating that the autoencoder does find differences during the production process. Similar for the false positives with a low fill duration. Apparently for these samples something has happened which makes the model think it is an anomaly.

In order to further explore why the model makes a certain decision. The values of the raw material usage and machine characteristics at an arbitrary point in time are explored. Further studies were performed for the raw material usage and machine characteristics after one hour. The choice for looking at one hour has been made due to the attention weight plots and SHAP values. Results indicate easily two clusters can be made, one dense cluster which follows standard process and one less dense which deviates from the standard process. Results for raw materials shows a cluster of samples where each combination of features used less materials or energy. The model predicts the samples of this cluster as an anomaly because these samples have higher reconstruction errors on this period. On the other hand, a more dense cluster is found, for which the samples of this cluster used more resources after one hour. These samples followed the standard production process and a result the deep hybrid anomaly detection model classifies these samples as normal.

### 8.3 Concluding Remarks

The problem was known to be very challenging because during the data exploration it was shown that the patterns of the in specification limit batches were not different from out of specification limit batches. Moreover, the problem was framed such that an early prediction was made, whereas the label of the batch is only assigned towards the end of the sequence. It is possible for the batches which currently remain undetected that the actual deviation took place later during the sequence. Additionally, the unknown raw material characteristics are also expected to affect the prediction performance. Although the detection rate of the best performing model was relatively low, model evaluation still revealed that the detection of an incorrect batch is related to any disturbances during filling the conche machine. One possible explanation for this might include that the quality of the raw materials might deteriorate if the filling process is disturbed.

### 9 Conclusion

Food quality management is known to be difficult because each disturbance is easily propagated throughout the process and affects the quality of the final product. Variability in chocolate quality causes problems downstream the manufacturing line. Hence, one of the main objectives of food processing operations is to damp the variability of the inputs in order to obtain consistent objective quality. Literature states chocolate manufactures require an efficient and reliable product and quality control method. This research proposes a deep hybrid model for early detecting anomalous behaviour of a faulty chocolate batch. The machine learning model uses time series process data and is capable of alarming whenever the chocolate production process has high chance of becoming out of control. This section summarizes the main findings of the research by answering the research question as proposed in Chapter 1. All chapters in this report provide an extensive answer to the sub questions. This section provides a brief summary of the findings, which will collectively answer the main research question:

What machine learning model can be developed to learn the influential factors of the quality during the production of Chocolate?

### 9.1 Main Findings

Online monitoring and process control of chocolate production is known to be challenging due to the crystallization within the process. The physical properties of chocolate consist of non-linear characteristics. As a result, the current control practice of Mars is reactive and relies heavily on the judgement of operators. Mars current practice measures the viscosity, yield, fat content and moisture at the end of the production cycle using expensive laboratory equipment and is performed using manual monitoring the process. As such, Mars can only intervene the production process with certainty when the actual values of the chocolate properties are known, which can further delay the production process. Besides the current practice, the literature proposes to utilize advanced online sensor equipment, scientific models or neural networks to monitor the quality of the chocolate. The latter two both require manual sampling or require input from advanced online sensors. The manual samples limit the applicability for online process control, whereas advanced online sensor equipment may not be applicable for large scale plants with multiple machines due to its large investment. Additionally, neural networks seem to be capable of predicting rheology of dough at the end of the production process purely based on the power of the engine. However, from a business perspective, an early prediction is required. The available literature does thus not provide a suitable approach. As such, this research extends current literature by making an early prediction based on early process log data.

Further, the literature describes process parameters such as particle size distribution, fat content, lecithin, temperature and conching time can all be used to control chocolate properties, while reducing the production costs and assuring quality. Moreover, the power curve of the main engine seemed an important predictor for the rheology of dough. During the data gathering, issues occurred mainly due to infrequent sampling rates or the inability to link different data sources and as a consequence data regarding the particle size distribution and properties of the used fats and lecithin are unavailable. Resulting in an available feature set of which all are related to engine characteristics or raw material usage over time. The exploratory data analysis showed that the data is highly imbalanced and the little anomalous sample set limits the modeling possibilities. Additionally the data exploration showed little differences between the patterns. As a result it is unknown whenever the fault occurs. The limited anomalous sample size together with little differences of the patterns makes the faced problem additionally challenging. As a result, anomaly detection methods for time series were trained. Comparing the results of the anomaly detection methods against a supervised LSTM classifier validated this choice. By applying anomaly detection during the chocolate production, the time-series anomaly detection methods are applied in a new context. Further, this research extends current anomaly time series detection literature by combining different autoencoders with supervised learning models.

Chocolate making is known as a complex process. Training autoencoders exclusively on early batch process data was capable of learning the normal processing behaviour. Normal behaviour is defined as the chocolate process data for which the first measured chocolate properties were in between specification limits. Due to the little differences between correct chocolate batches and chocolate batches with their properties outside specification limits, the trained autoencoders were only capable of detecting a small proportion of the faulty batches with low precision. Besides the low precision, large variance in the model performance was observed which indicates an unstable model with little generalization capabilities. Further, inspections of the miss classifications gave birth to the doubt into the quality of the chosen specification limits. At Mars, the specification limits were chosen empirically and purely based on domain knowledge. Additionally, quality technicians mention the manually influence of the operators on the final sample properties. Changing the soft specification limits to the harder control limits improved the anomaly detection capability of the investigated autoencoders and deep hybrid models. Overall the best performing model detects out of control chocolate batches by combining the output of an Attention autoencoder with a Random Forest. It obtains on average a reasonable precision and recall. Changing the labels to out of control anomalies further reduced the variance of the performance metrics and thus improved the robustness of the model.

The best performing model is a semi-supervised model which consists of an unsupervised attention based autoencoder combined with a supervised Random Forest binary classification model. Although it is uncertain whenever the actual fault occurs, important features were missing and little differences between patterns exist, based on the sensitivity analysis, the final model can still alarm an operator with almost 70 percent precision and detects about 40 percent of all faulty batches. This demonstrates the capability of neural networks to learn the desired processing behaviour. Moreover, the attention mechanism and supervised learning method both facilitated model interpretation. The attention mechanism can be used to visualize important minutes for reconstructing the time series sequence, whereas SHAP values can be used to interpret the predictions from both a global and local perspective. The former provides an importance for each feature related to the target variable, and the latter increases the transparency of individual predictions based on their feature values.

### 9.2 Business Recommendation

Machine learning algorithms are only as good as the data they are fed. One of the drawbacks of this research concerns that the data related to the chocolate production was stored on different databases. Literature and interviews revealed that certain features are expected to influence the chocolate properties, but due to the different storage locations these features remain unexplored. This availability of the data features is expected to limit the obtained model performance. Therefore, it is recommended to adapt current data storage methods to enable the linking between databases. Moreover, we recommend developing a new database system which links all different systems. After improving the data availability, the model could be reconfigured and implemented. Implementing such a model could increase the efficiency of the process and reduce operator workload. Currently, Mars relies on the operators to notice faulty processing behaviour of a conche. Each operator must monitor multiple conches from a milling group, the anomaly detection model could stress attention towards the actual batch which is expected to become faulty. However, implementation of the developed model is not trivial and should not be underestimated.

In terms of modeling the differences between good and faulty batches, we recommend Mars to reinvestigate the current specification and control limits. Data exploration and results of the anomaly detection methods revealed a chocolate batch which is out of control, but within specification limit, shows no real different characteristics from the out of specification batches. As a result, detecting differences for facilitating process control with the current data is proven to be a difficult task.

The recommendation regarding the filling duration is twofold. First, the deep hybrid detection model with attention mechanism highlighted the importance of reducing the disturbances during the filling phase. Therefore the first recommendation concerns that Mars should only start filling the conche machine with its raw materials if it is sure it can be finished under 60 minutes. As such the quality of raw materials will not deteriorate while being within the conche. Secondly, discussions with quality technicians revealed disturbances during the filling phase are often manual interventions, but currently it has not been logged why a certain disturbance happened. As an effect these disturbances could not have been investigated and therefore in order to improve the analysis we recommend to log certain disturbances.

### 9.3 Limitations and Future Research

Finally, the research is concluded by describing the limitations of this study and specifying the directions for future research:

The applicability of the anomaly detection process model to alarm for faulty process batches depends on the quality of data and number of faulty samples available at Mars. Although the deep hybrid anomaly detection method for out-of-control anomalies obtains reasonable performance. A large part of the faulty chocolate batches remain undetected. Literature states that the final chocolate quality is affected by the quality of its input. Moreover, currently unsupervised autoencoders were used as a model to detect faulty samples. The choice for autoencoders was based on the limited availability of faulty samples. Autoencoders enable to learn exclusively from normal data and the autoencoders trained on controlled chocolate batches demonstrated the capability of neural networks to learn the desired processing behaviour. However, the autoencoders eliminated the possibility to classify the actual fault. The performance of autoencoders might also be sub-optimal. Literature state the objective function of autoencoders focuses on dimensionality reduction, rather than anomaly detection. Therefore, the representations of the autoencoders are a generic summarization of the underlying regularities which are not optimized for anomaly detection. In case more samples are available, classification neural networks might directly be trained with the objective to classify a fault. Moreover, machine learning algorithms should finally be evaluated using performance measures that represent costs in the real world. Due to the anomaly detection direction, no translations in terms of costs of savings can be made. Not having this information complicates the model's evaluation in terms of saved costs and performance.

During the model training and evaluation we observed large variances. The test performance of the anomaly threshold for out of specification anomalies showed large variance among different validation and test splits. Additionally, large variances were observed during the parameter optimization of the supervised models within the deep hybrid models. These observations indicate that the used splits are an unrepresentative sample of the data from the domain. Implying that the sample size is too small or the examples in the sample do not effectively cover the cases observed in the broader domain. In this study 1917 samples were available, where each sample consists of 21 features measurements over time. Obtaining a larger and more representative sample set can be a solution. Further, in this study, the samples were split into such sets by first splitting exclusively the normal samples into a train, validation and test set. Afterwards, the anomalous samples were split into validation and test set. The splitting was thus performed based on the labels in a stratified manner. Alternatively, other discriminating methods which consider the population characteristics in preparing the training, validation and test could be used. Since, the used splits were unrepresentative, using a stratified splits on the input variables could be an attempt to maintain the population means and standard deviations and could be explored in future research.

Moreover, there are a few limitations regarding the modeled autoencoders. Currently, the autoencoders are regularized using under-complete latent representation. Autoencoders are forced to learn important regularities of normal behaviour by incorporating a bottleneck. However, using a bottleneck is one way of regularizing an autoencoder to force them to learn the exact features, but is not a requirement. Over-complete autoencoders, with higher dimension latent representations combined with regularization can also learn sufficient relevant features. Future research could investigate whether higher performance could be obtained using over-complete autoencoders. However, when there are more nodes in the hidden layer than there are inputs, an autoencoder is risking to learn the identity function, meaning that output equals the input. Further, the current applied attention mechanism stressed attention on the time axis, in a similar manner future research could explore putting attention on the features. Additionally, this research investigated the use of variational autoencoders for detecting anomalies. In order to have a fair comparison between the different autoencoder types, a threshold on the reconstruction error was set which determines whether the sequence was anomalous or not. Compared to the other two autoencoder types, the variational autoencoders yielded worse performance. Literature explained variational autoencoders can also be used to output a reconstruction probability. As a result, the full potential of variational autoencoders has not been utilized and future research could investigate whether the reconstruction probability yields better performance.

### Hardware

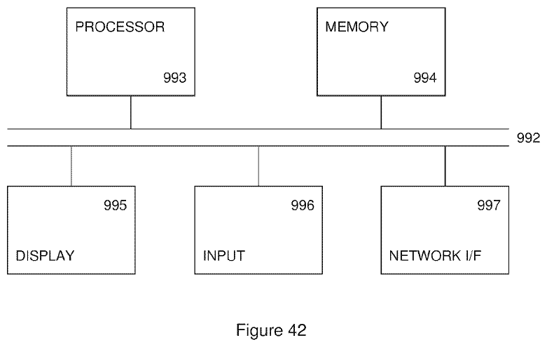

FIG. 42 is a block diagram of a computing device, such as a data storage server or PC or laptop, which embodies the present invention and which may be used to implement aspects of the methods for enhancing object detection in digital images, as described herein. The computing device comprises a processor 993, and memory 994. Optionally, the computing device also includes a network interface 997 for communication with other computing devices.

For example, an embodiment may be composed of a network of such computing devices. Optionally, the computing device also includes one or more input mechanisms such as keyboard and mouse 996, and a display unit such as one or more monitors 995. The components are connectable to one another via a bus 992.

The memory 994 may include a computer readable medium, a term which may refer to a single medium or multiple media (e.g., a centralised or distributed database and/or associated caches and servers) configured to carry computer-executable instructions or have data structures stored thereon. Computer-executable instructions may include, for example, instructions and data accessible by and causing a general-purpose computer, special purpose computer, or special purpose processing device (e.g., one or more processors) to perform one or more functions or operations. Thus, the term “computer-readable storage medium” may also include any medium that is capable of storing, encoding, or carrying a set of instructions for execution by the machine and that cause the machine to perform any one or more of the methods of the present disclosure. The term “computer-readable storage medium” may accordingly be taken to include, but not be limited to, solid-state memories, optical media, and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device 400 and to execute processing operations, for example executing code stored in the memory 404 to implement the various different functions of the active learning method, as described here and in the claims.

The memory 994 may store data being read and written by the processor 993, for example data from training or classification tasks executing on the processor 993. As referred to herein, a processor 993 may include one or more general-purpose processing devices such as a microprocessor, central processing unit, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 993 may also include one or more special-purpose processing devices such as an application specific integrated circuit (ASIC), a field programmable gate array (FPGA), a digital signal processor (DSP), network processor, or the like. In one or more embodiments, a processor 993 is configured to execute instructions for performing the operations and steps discussed herein.

The network interface (network I/F) 997 may be connected to a network, such as the Internet, and is connectable to other computing devices via the network. The network I/F 997 may control data input/output from/to other apparatuses via the network.

Methods embodying aspects of the present invention may be carried out on a computing device such as that illustrated in FIG. 42. Such a computing device need not have every component illustrated in FIG. 42 and may be composed of a subset of those components. A method embodying aspects of the present invention may be carried out by a single computing device in communication with one or more data storage servers via a network or by a plurality of computing devices operating in cooperation with one another. Cloud services implementing computing devices may be deployed.

1. A computer-implemented method of predicting quality of a food product sample after a mixing process, based on properties of the food product, the method comprising:
building a hybrid model by:
training an autoencoder in an unsupervised learning step using historical process data of food product samples, and
training a supervised model in a supervised learning step using the output of the autoencoder; and
predicting the quality of the food product by inputting process data of current samples into the hybrid model and classifying the current samples.

2. A method according to claim 1, further comprising using sensors to capture online and/or inline process data from a food manufacturing line.

3. A method according to claim 1, wherein the process data includes raw material quantity data and/or mixing engine characteristics.

4. A method according to claim 1, wherein the historical process data and/or the process data of current samples is truncated.

5. A method according to claim 1, further comprising alerting an operator of an expected anomalous batch of food product if one or more current samples is classified as anomalous.

6. A method according to claim 1 wherein the autoencoder includes an attention mechanism.

7. A method according to claim 1, wherein the supervised learning model is a random forest binary classification model.

8. A method according to claim 1, wherein the autoencoder is trained in a semi-supervised manner, first training the model unsupervised on purely normal samples, and then using a validation set comprising anomalous samples.

9. A method according to claim 1, wherein the food product is confectionary such as chocolate or caramel or cookie dough and preferably wherein the food product is chocolate and the mixing process is conching.

10. A method according to claim 1, wherein the properties used in the determination of whether samples are labelled normal or anomalous are any or all of yield stress (Pa), viscosity (pa·s), fat content (%) and moisture (%) of the food product being mixed, and preferably wherein each of yield, viscosity, fat content and moisture must be within a given range to give a normal sample.

11. A method according to claim 1, wherein the output of the autoencoder comprises a reconstruction error of the autoencoder.

12. A method according to claim 1 wherein predicting the quality of the food product by inputting process data of current samples into the hybrid model and classifying the samples comprises:
inputting process data of current samples to the autoencoder, wherein the autoencoder is configured to compress the input process data to a latent space, and to reconstruct the process data from the latent space;
generating a reconstruction error between the input process data and the reconstructed process data;
inputting the reconstruction error to the supervised model, wherein the supervised model is configured to process the reconstruction error according to supervised model parameters set during the supervised learning step; and
obtaining from the supervised model an output comprising a predicted value of a measure indicating quality of the food product.

13. A method according to claim 1, wherein training a supervised model in a supervised learning step using the output of the autoencoder comprises:
assembling a training data set comprising, for historical process data of food product samples, outputs from the autoencoder and labels corresponding to the outputs and comprising values of a measure indicating quality of the food product; and
using the assembled training data set to update trainable parameters of the supervised model.

14. A method according to claim 13, wherein using the assembled training data set to update trainable parameters of the supervised model comprises:
inputting the outputs from the autoencoder to the supervised model and obtaining from the supervised model outputs comprising a predicted value of a measure indicating quality of the food product; and
updating trainable parameters of the supervised model so as to minimise a loss function based on a difference between the output of the supervised model and the labels of the training data set.

15. A computer-implemented semi-supervised hybrid anomaly detection method of classifying a food product sample after a mixing process, based on properties of the food product, the method comprising:
accessing a hybrid model created by:
training an autoencoder in an unsupervised learning step using historical process data of food product samples, and
training a supervised-model in a supervised learning step using the output of the autoencoder; and
inputting process data of current samples into the hybrid model to classify the current samples.

16. A computer-implemented method of training a hybrid model to predict quality of a food product sample after a mixing process, based on properties of the food product, the method comprising:
training an autoencoder in an unsupervised learning step using historical process data of food product samples; and
training a supervised model to classify food product samples in a supervised learning step using the output of the autoencoder.

17. A data processing apparatus comprising a memory and a processor configured to carry out the method of claim 1.

18. A computer program comprising instructions, which, when the program is executed by a computer, cause the computer to carry out the method of claim 1.

19. A computer-readable medium comprising instructions which, when executed by a computer, cause the computer to carry out the method of-claim 1.
